(19) United States
(12) Patent Application Publication
(10) Pub. No.: US 2021/0198681 A1
(43) Pub. Date: Jul. 1, 2021

(54) ARTIFICIAL MARKER ALLELE

(71) Applicant: KWS SAAT SE & Co. KGaA, Einbeck (DE)

(72) Inventor: Dietrich BORCHARDT, Einbeck (DE)

(73) Assignee: KWS SAAT SE & Co. KGaA, Einbeck (DE)

(21) Appl. No.: 17/056,043

(22) PCT Filed: May 23, 2019
(86) PCT No.: PCT/EP2019/063404

§ 371 Date: Nov. 17, 2020

(30) Foreign Application Priority Data
May 24, 2018 (EP) 18174057.2

(51) Int. Cl. C12N 15/82

(52) U.S. Cl. CPC C12N 15/8209

(57) ABSTRACT

This invention relates to a method for making an artificial marker allele for the identification of a nucleic acid of interest in an organism. The invention also relates to determining the presence of a nucleic acid of interest in a mixed population and a method for introgressing a nucleic acid of interest into a population. The invention also relates to organisms, particularly plants and seeds, comprising such a marker allele and to various uses for the artificial marker allele.

### TECHNICAL FIELD

This invention relates to the field of biotechnology. More specifically, the invention relates to a method for making an artificial marker allele for the identification of a nucleic acid of interest in an organism. The invention also relates to determining the presence of a nucleic acid of interest in a mixed population and a method for introgressing a nucleic acid of interest into a population. The invention also relates to organisms, particularly plants and seeds, comprising an artificial marker allele and to various uses for the artificial marker allele.

### BACKGROUND

Plant breeding has made remarkable progress in increasing crop yields for over a century. Nevertheless, plant breeders constantly face new challenges. Changes in agricultural practices create the need for developing genotypes with new agronomic characteristics. New fungal and insect pests continually evolve and overcome existing host-plant resistance. New land areas are regularly being used for farming, exposing plants to altered growing conditions. Finally, a rising global population will require increased crop for food production. Thus, the task of increasing crop yields represents an unprecedented challenge for plant breeders and agricultural scientists.

Plant breeding will play a key role in the coordinated effort for providing solutions to the above problems. Given the context of current yield trends, predicted population growth and pressure on the environment, traits relating to yield stability and sustainability are a major focus of plant breeding efforts. These traits include durable disease resistance, abiotic stress tolerance and nutrient- and water-use efficiency.

Despite optimism about continued yield improvement from conventional breeding, new biotechnological solutions will be needed to maximize the probability of success. One area of biotechnology, namely DNA marker technology, derived from research in molecular genetics and genomics, offers great promise for plant breeding. Owing to genetic linkage, DNA markers can be used to detect the presence of allelic variation in the genes underlying a desired trait. By using DNA markers to assist in plant breeding, efficiency and precision could be greatly increased. The use of DNA markers in plant breeding is called marker-assisted selection (MAS) and is a component of the new discipline of ‘molecular breeding’.

Over the last two decades, the use of DNA marker technology in plant breeding has dramatically increased. However, the use of marker assisted breeding and the identification of suitable markers is a laborious and time-consuming process. Furthermore, marker assisted breeding is limited because plant genomes are richly dispersed with repetitive sequences which significantly obstruct the possibility of the development and use of diagnostic markers. Especially for crops with large genome sizes, the identification of low copy number DNA segments can be highly challenging. Furthermore, in many cases only DNA polymorphisms with extremely tight linkage to the trait gene or even the causal polymorphism itself can be exploited to be converted into useful DNA markers.

EP 2 342 337 B1 describes a method of introducing unique, artificial and selectable markers at targeted regions instead of identifying and exploiting naturally occurring polymorphisms. The strategy described is based on identifying and selecting a section of DNA that is closely linked to the trait(s) of interest and converting this section into a selectable marker by inserting a single nucleotide polymorphism (SNP) into a substantially conserved nucleotide composition of this DNA section. The method described in EP 2 342 337 B1 however suffers from the drawbacks of being time-consuming and laborious and generating a marker of low sensitivity and reliability, making the resulting markers unsuitable for quality control purposes, for example.

It would therefore be advantageous to be able to provide artificial marker alleles and methods for the production of the same which overcome the aforementioned problems.

### SUMMARY OF THE INVENTION

The present invention overcomes these problems by providing artificial InDel marker alleles having increased sensitivity and reliability that can be used in particular for quality control applications.

According to a first aspect of the present invention, there is provided a method for making an artificial marker allele for the identification of a nucleic acid of interest, preferably encoding a polypeptide conferring a trait of interest, in an organism, the method comprising:

(a) identifying at least one genomic locus in the genome of the organism, which is genetically linked to the nucleic acid of interest, and
(b) introducing at least one InDel into the at least one genomic locus,
thereby making a marker allele which is inheritable to subsequent generations of the organism along with the nucleic acid of interest.

According to a second aspect of the present invention, there is provided a method for determining the presence of a nucleic acid of interest, preferably encoding a polypeptide conferring a trait of interest, in a mixed population of individuals comprising the nucleic acid of interest and individuals not comprising the nucleic acid of interest, said method comprising detection of an artificial marker allele as defined in the first aspect of the invention using at least one molecular marker specific for the artificial marker allele and/or at least one molecular marker specific for the wild type genomic locus.

According to a third aspect of the present invention, there is provided a method for assessing the homogeneity of a population of individuals comprising a nucleic acid of interest, preferably encoding a polypeptide conferring a trait of interest, said method comprising detection of an artificial marker allele as defined in the first aspect of the invention and determining homogeneity in the population by using at least one molecular marker specific for the artificial marker allele and/or at least one molecular marker specific for the wild type genomic locus, wherein the detection of the wild type genomic locus indicates heterogenous distribution of individuals comprising the nucleic acid of interest in the population.

According to a fourth aspect of the present invention, there is provided a method for introgressing a nucleic acid of interest, preferably encoding a polypeptide conferring a trait of interest, to a population of individuals, comprising the steps of:
(i) making an artificial marker allele according to the first aspect of the invention in a donor organism comprising the nucleic acid of interest;
(ii) crossing said donor organism with a recipient organism of the same species not comprising the nucleic acid of interest to generate progeny of heterogenous genetic composition;
(iii) backcrossing/selfing and selection for the presence of the artificial marker allele to obtain progeny of homozygous genetic composition, which comprise the nucleic acid of interest in the background of the recipient organism,
(iv) optionally, repeating step (iii) at least once, preferably several times.

Step (iii) of the method is based on detection using at least one molecular marker specific for detection of the presence of the artificial marker allele in the progeny and/or at least one molecular marker specific for detection of the absence of the artificial marker allele in the progeny.

The recipient organism may be a plant, an animal, a microorganism or a fungus, preferably a plant, more preferably a plant of an elite line, a wild type plant, a mutant plant, a gene-edited or a base-edited plant or a transgenic plant.

According to a fifth aspect of the present invention, there is provided a method for making an artificial marker allele comprising designing one or more genotype-specific InDels and introducing said InDels into a genomic locus in the genome of an organism, wherein the genomic locus is genetically linked to a nucleic acid of interest, preferably encoding a polypeptide conferring a trait of interest. Also provided is an artificial marker allele comprising at least one genotype-specific InDel obtainable by such method.

According to a sixth aspect of the present invention, there is provided use of an artificial marker allele according to the fifth aspect or use of an artificial marker allele obtainable by a method according to the first aspect of the present invention in marker assisted breeding.

Also provided is the use of a programmable nuclease for the generation of an artificial marker allele according to the first aspect of the present invention for the identification of a nucleic acid of interest in the genome of an organism. The programmable nuclease may be selected from CRISPR nuclease systems, zinc finger nucleases, TALENs, meganucleases, or base editors.

According to a seventh aspect of the present invention, there is provided an organism, preferably a plant or a seed thereof, comprising an artificial marker allele obtainable by a method according to the first aspect or comprising an artificial marker allele according to the fifth aspect.

### DETAILED DESCRIPTION

The first aspect of the present invention provides a method for making an artificial marker allele for the identification of a nucleic acid of interest, preferably encoding a polypeptide conferring a trait of interest, in an organism, the method comprising:

(a) identifying at least one genomic locus in the genome of the organism, which is genetically linked to the nucleic acid of interest, and
(b) introducing at least one InDel into the at least one genomic locus,
thereby making a marker allele which is inheritable to subsequent generations of the organism along with the nucleic acid of interest.

The nucleic acid of interest preferably encodes a polypeptide encoding a trait of interest. The trait may be a phenotypic trait and may be observable phenotypically, e.g., by the naked eye or by other means, such as microscopy, through biochemical analysis, genomic analysis, transcriptional profiling etc. The phenotype may be attributed to a single gene or genetic locus or may result from the action of several genes. Typical traits in the genome of a plant of economic importance include yield-related traits, including lodging resistance, flowering time, shattering resistance, seed color, endosperm composition, nutritional content, herbicide resistance, including resistance to glyphosate, glufosinate/phosphinotricin, hygromycin (hyg), protoporphyrinogen oxidase (PPO) inhibitors, ALS inhibitors, and Dicamba, disease resistance, including viral resistance, fungal resistance, bacterial resistance, or insect resistance, resistance or tolerance to abiotic stress, including drought stress, osmotic stress, heat stress, cold stress, oxidative stress, heavy metal stress, nitrogen deficiency, phosphate deficiency, salt stress or waterlogging and nutrient- and water-use efficiency, male sterility. In a preferred embodiment of the invention, a trait of interest may be artificially introduced into a nucleic acid of interest by means of gene-editing (GE) based or base editor based gene modification based on gene-editing (GE) by means of a programmable nuclease or nickase, based on base editing by means of a base editor or based on a combination thereof.

An “allele” as used herein refers to a variant form of a nucleic acid sequence or gene at a particular genomic locus and the term “artificial marker allele” as used herein is taken to mean an artificially created unique allele generally not found in nature in an organism in question. The “artificial marker allele” in the context of the present invention is genetically linked to a nucleic acid of interest which is associated with a desired trait. The “artificial marker allele” as used herein therefore refers to a nucleotide polymorphism which can be used for the identification of a nucleic acid of interest associated with a trait of interest in the genome of an organism.

The first step of the method for making an artificial marker allele comprises identifying at least one genomic locus in the genome of the organism that is genetically linked to the nucleic acid of interest. Such a genomic locus is one which is unique within the genome of an organism and highly conserved across different genotypes of the organism. A skilled person in the field of animal or plant breeding will appreciate what is meant by the term “highly conserved” in the context of the present invention. In particular, the term “highly conserved” as used herein refers to a genomic sequence, preferably between 100 and 200 bp in length, which shares at least 90%, 90.5%, 91%, 91.5%, 92%, 92.5%, 93%, 93.5%, 94%, 94.5% 95%, 95.5%, 96%, 96.5%, 97%, 97.5%, 98%, 98.5%, 99%, 99.5% or 100% sequence identity across different genotypes of the organism. As used herein, “genotype” refers to the genetic constitution of an individual or group of individuals at one or more genetic loci. The genotype of an individual or a group of individuals is the sum of all genes and determines its phenotype. When referring to conservation across different genotypes of an organism, this may be conservation across different individuals in a population of a given species, cultivars or races of the organism. “Cultivar” and “variety” are used interchangeably herein to mean a group of plants within a species, for example B. vulgaris, that share certain genetic traits resulting in the same phenotype that separate them from other possible varieties within that species. Cultivars can be inbreds or hybrids, as applicable for the crop in question.

To identify a highly conserved genomic locus across different genotypes, detailed sequence analysis in a group of individuals is carried out to identify a region of approximately 100 to 200 base pairs (bp). The identified region must be unique within the target genome to allow specific insertion of the InDel by genome editing or base editing. The highly conserved genomic locus then allows general usage of the marker in the broadest possible range of genotypes and genetic background.

The genomic locus is ideally positioned outside of any coding region (exceptionally there may be reason to select a coding region), splicing signal or regulatory element of the nucleic acid of interest, 3′UTRs, 5′UTRs, introns, miRNAs, non-coding RNAs and any other possible features. These precautions are taken because genomic interaction cannot be excluded. The promoter region of a gene is usually not very well characterized, therefore a location in 3′ direction of the target gene is preferred. However, where location is in the 5′ region of a gene is favored, a promoter length of 1000 bp is assumed and will not be selected for introduction of the at least one InDel.

Furthermore, the genomic locus should preferably be in the physical vicinity and complete linkage disequilibrium (LD) to the nucleic acid of interest to avoid separation of the artificial marker allele from the nucleic acid of interest in the course of recombination. The term “linkage disequilibrium” (LD) refers to a non-random segregation of genetic loci or traits (or both) and implies that the relevant loci are within sufficient physical and/or genetic proximity along a length of a chromosome so that they segregate together with greater than random (i.e., non-random) frequency.

The genomic locus is closely linked to the nucleic acid of interest such that when an InDel is introduced into the genomic locus, so as to create an artificial marker allele, the marker allele is inheritable to subsequent generations of the organism along with the nucleic acid of interest. The genomic locus is ideally positioned in a region flanking the nucleic acid of interest and is preferably located at the 3′ end of the nucleic acid of interest. The region flanking the nucleic acid of interest is preferably at a distance of at least 2 cM, 1 cM, 0.5 cM, 0.1 cM, 0.09 cM, 0.08 cM, 0.07 cM, 0.06 cM, 0.05 cM, 0.04 cM, 0.03 cM, 0.02 cM 0.01 cM, 0.009 cM, 0.008 cM, 0.007 cM, 0.006 cM, 0.005 cM, 0.004 cM, 0.003 cM, 0.002 cM 0.001 cM, 0.0009 cM, 0.0008 cM, 0.0007 cM, 0.0006 cM, 0.0005 cM, 0.0004 cM, 0.0003 cM, 0.0002 cM or 0.0001 cM from the nucleic acid of interest or at a distance anywhere in between the above values. “cM” as used herein defines the distance between two loci on a chromosome and is a measurement of recombination frequency well known in the art.

The terms “flanking region . . . ” or “region flanking . . . ” are used interchangeably herein and refer to a nucleic acid sequence of a predetermined genomic locus which is genetically linked to a nucleic acid of interest into which the at least on InDel is inserted to generate an artificial InDel marker allele.

Alternatively, the genomic locus may be within the nucleic acid of interest itself. When the genomic locus is located within the nucleic acid of interest, the genomic locus should preferably be positioned outside of any coding region, splicing signal or regulatory element of the nucleic acid of interest, 3′UTRs, 5′UTRs, introns, miRNAs, non-coding RNAs and the like, so that when the at least one InDel is introduced into the genomic locus it does not cause a loss of function.

The nucleotide sequence of the genomic locus obtained after insertion of the at least one InDel, i.e. the obtained artificial marker allele, is unique within the genome of the organism, as far as can be determined, meaning that it does not occur or only very rarely occurs in the germplasm of the organism in question. The resulting organism thus contains a specifically introduced alteration in its genetic sequence that is closely linked to the nucleic acid of interest, which preferably encodes a polypeptide conferring a trait of interest. This specifically introduced InDel (which creates an artificial marker allele) can now be used and assayed in any conventional way in marker-assisted breeding, and as further described herein.

The term “germplasm”, as used herein, refers to genetic material with a specific molecular makeup that provides for some or all of the hereditary qualities of an organism or cell culture and collections of that material. Breeders use the term “germplasm” to indicate their collection of genetic material from e.g. wild type species, elite or domestic breeding lines from which they can draw to create varieties or races. As used herein, “germplasm” may be any living genetic resource including but not limited to cells, seeds or tissues from which new plants may be grown, or plant parts, such as leaves, stems, pollen, ovules, or cells that can be cultured into a whole plant.

The “organism” is preferably a plant, but may also be an animal, fungus or microorganism. The term “plant” as used herein refers to whole plants, ancestors and progeny thereof and to plant parts. Plant parts may include seeds, tissues, cells, organs, leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, gametophytes, spores and cuttings. The term “plant” as used herein also comprises germplasm of a plant which can be cultured into whole plants or plant parts. Progeny and ancestor plants can be from any filial generation, e.g. P, F1, F2, F3 and so on and any plant resulting from backcrossing therefrom.

The plant may be any plant and may, for example, be selected from Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

The second step in the method for making an artificial marker allele comprises introducing at least one InDel into the at least one genomic locus.

An “InDel” or “InDel marker” as defined herein is taken to mean at least one nucleotide insertion and/or at least one nucleotide deletion in the genomic locus within the genome of an organism. The at least one nucleotide insertion is also referred to herein as an “insertion marker” and the at least one nucleotide deletion is also referred to herein as a “deletion marker”.

An “InDel” in the context of the present invention refers to an insertion and/or deletion of at least one nucleotide in the nucleotide sequence of a predetermined genomic locus, thereby altering the length of the nucleotide sequence of the genomic locus by at least one nucleotide. An “InDel” in the context of the present invention therefore refers to the incorporation of at least one additional nucleotide into an endogenous nucleotide sequence or the removal of at least one nucleotide from an endogenous nucleotide sequence. In contrast to an InDel, a “single nucleotide polymorphism” (SNP) means a sequence variation that occurs when a single nucleotide (A, C, T or G) in the genomic sequence is altered. A SNP is a substitution or replacement of a single nucleotide within a given nucleotide sequence, which leaves the length of the nucleotide sequence unchanged.

In a preferred embodiment of the invention, the at least one InDel comprises more than one nucleotide insertion and/or more than one nucleotide deletion. The at least one InDel may comprise an insertion of between 1 and 60 base pairs of a sequence which is non-homologous to the genome of the organism in which the at least one InDel is to be introduced. Optionally, the InDel may comprise an insertion of more than 60 base pairs of a sequence which is non-homologous to the genome of the organism in which the at least one InDel is to be introduced. The insertion may optionally comprise or consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 of a sequence which is non-homologous to the genome of the organism in which said at least one InDel is introduced. Preferably, the insertion comprises or consists of a nucleotide sequence of at least 9, 10, 11, 12, 13, 14, 15, 16, 17, 18, 19 or 20 base pairs of a sequence which is non-homologous to the genome of the organism in which said at least one InDel is introduced. The term “non-homologous” in this context means that the insertion is unique and does not share homology to a nucleic acid sequence in the genome of the organism which might potentially result in the incorporation of the insertion into an undesired genomic location due to homology-directed repair. The term “non-homologous” in the present context further means that the insertion when introduced into the genomic locus results in an artificial marker allele which is unique within the genome of the organism in question.

In the context of the method of making an artificial marker allele, the insertion and its flanking region in the predetermined genomic locus need to be evaluated and selected for optimal assay design, meaning that they must be singular and non-repetitive in the genome of a given organism. Furthermore, the insertion and its flanking region should exhibit one or more of the following characteristics: approximately 50% GC content, balanced distribution between G/C and A/T bases, reduced chance of secondary structures. The insertion of at least one nucleotide in the flanking region of a predetermined genomic locus should result in an insertion marker allele which is monomorphic, i.e. unique, across different genotypes of the organism. This analysis is carried out through iterative and repeated analysis of short sequences using standard bioinformatic tools and sequencing approaches.

Furthermore, the flanking region should be monomorphic in the gene pool of the organism meaning that it is highly conserved between different genotypes of the organism.

In a further embodiment of the present invention, the at least one InDel may additionally or alternatively comprise or consist of a deletion of between 1 and 60 base pairs of a sequence in the genomic locus in the genome of the organism. Optionally, the deletion is of more than 60 base pairs of a sequence in the genomic locus in the genome of the organism. The deletion may be of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 base pairs. Preferably, the deletion comprises or consists of a nucleotide sequence of at least 9, 10, 11, 12, 13, 14, 15, 16, 17, 18, 19 or 20 base pairs.

The deletion selected is one which does not result in a loss of function in the gene or genomic region. For example, the deletion marker is preferably located outside a gene associated with the desired trait of interest and/or is located in a non-coding region so as to avoid any loss-of-function. One skilled in the art would readily appreciate which genomic regions to avoid when designing a suitable deletion marker.

Furthermore, the deletion and its flanking region in the predetermined genomic locus need to be selected for optimal assay design, meaning that they must be singular and non-repetitive in the genome of a given organism and exhibit one or more of the following characteristics: approximately 50% GC content, balanced distribution between G/C and A/T bases, reduced chance of secondary DNA structures. The deletion of at least one nucleotide in the flanking region in the predetermined genomic locus should result in a deletion marker allele which is monomorphic across different genotypes of the organism. This analysis is carried out through iterative and repeated analysis of short sequences using standard bioinformatic tools.

A “balanced distribution between G/C and A/T bases” refers to a content of 40%-55% GC and respective A/T, i.e. 60%-45% depending on the actual GC content. The distribution may be 40% G/C and 60% A/T, 41% G/C and 59% A/T, 42% G/C and 58% A/T, 43% G/C and 57% A/T, 44% G/C and 56% A/T, 45% G/C and 55% A/T, 46% G/C and 54% A/T, 47% G/C and 53% A/T, 48% G/C and 52% A/T, 49% G/C and 51% A/T, 50% G/C and 50% A/T, 51% G/C and 49% A/T, 52% G/C and 48% A/T, 53% G/C and 47% A/T, 54% G/C and 46% A/T, and/or 55% G/C and 45% A/T. A balanced distribution between G/C and A/T bases effects the creation of secondary structures in the DNA at or adjacent to the predetermined locus, whereby such secondary structures influence the annealing of molecular markers. Ones skilled in the art is well-aware of this fact and is able to predict computational the suitability of a certain sequence for an optimal assay design.

Furthermore, the flanking region should be monomorphic, i.e. highly conserved in the gene pool of the organism meaning that it is highly conserved between different genotypes of the organism.

The insertion and/or deletion size can vary depending on the marker assays to be developed.

“Introducing” in the meaning of the present invention includes stable or transient integration by means of transformation including Agrobacterium-mediated transformation, transfection, microinjection, biolistic bombardment, insertion using gene editing technology like CRISPR systems (e.g. CRISPR/Cas, in particular CRISPR/Cas9 or CRISPR/Cpf1), CRISPR/CasX, or CRISPR/CasY), TALENs, zinc finger nucleases or meganucleases, homologous recombination optionally by means of one of the below mentioned gene editing technology including preferably a repair template, modification of a genomic locus using random or targeted mutagenesis like TILLING or mentioned gene editing technology, etc.

Preferably the at least one InDel may be introduced into the genomic locus using any known suitable mutagenesis methods for the introduction of nucleotide insertion(s) and/or deletion(s).

For example, the at least one InDel may be introduced using a programable nuclease or nickase. The programmable nuclease or nickase may be selected from any known gene editing (GE) tools, such as site-directed nucleases (SDNs), including CRISPR nuclease system, including a CRISPR/Cas9 system, a CRISPR/Cfp1 system, a CRISPR/CasX system, a CRISPR/CasY system, zinc-finger nucleases, TALENs, meganucleases and/or any combination, variant or catalytically active fragment thereof.

Site directed nucleases (SDNs) or nickases use a DNA cutting enzyme (nuclease) for the generation of the targeted (or site directed) DNA break. Variants of SDN applications are often categorized as SDN-1 (absence of a repair template), SDN-2 (gene editing by using DNA repair template) and SDN-3 (introduction of larger insertions/deletions by using DNA repair template) depending on the outcome of the DNA double strand break repair or the DNA single strand break repair.

Any programable nuclease or nickase may be used for the introduction of point mutations, insertions or deletions into the genome of an organism. The skilled person would readily be able to select a suitable technique based on the genomic sequence and the desired efficiency.

For example, point mutations may be generated by a classic SDN-1 approach (i.e. non-homologous end joining (NHEJ) to randomly insert/delete one or more bases to cause a point mutation). At the position where the point mutation is to be generated (or in close proximity thereto), the double strand is cleaved. The NHEJ pathway then repairs the double strand break, thereby randomly generating the desired point mutation. The selection of one particular point mutation would be difficult as a large number of plants would need to be screened, therefore, a SDN-2 approach (as detailed below) is preferred for introducing specific point mutations at a predetermined genomic location (i.e. homology directed repair (HDR) with repair template).

For the SDN-2 approach the DNA double strand is cleaved at a predetermined genomic location (or in close proximity thereto) where the point mutation is to be introduced. By adding a “repair template” with homologous flanking regions upstream and downstream of the cleavage site, the desired point mutation can be introduced by HDR. This increases the probability of obtaining the desired mutation.

In general, the approaches described for the generation of point mutations also work for the generation of deletions. In addition to the above approaches, it is possible to delete a desired sequence by generating two double strand breaks upstream and downstream of the sequence to be deleted. In the selection step it is then important to ensure that a precise cleavage has occurred.

The approaches described for the generation of point mutations also work for the generation of insertions. The SDN-2 approach is preferred for the generation of insertions, although the SDN-1 approach may also be useful for in certain circumstances.

A CRISPR nuclease system in this context describes a molecular complex comprising at least one small and individual guide RNA in combination with a Cas nuclease or another CRISPR nuclease like a Cpf1 nuclease (Zetsche et al. (2015); “Cpf1 is a single RNA-guided endonuclease of a class 2 CRISPR-Cas system”. Cell 163(3): 759-771) which can produce a specific DNA double-stranded break. The terms “CRISPR polypeptide”, “CRISPR endonuclease”, “CRISPR nuclease”, “CRISPR protein”, “CRISPR effector” or “CRISPR enzyme” are used interchangeably herein and refer to any naturally occurring or artificial amino acid sequence, or the nucleic acid sequence encoding the same, acting as site-specific DNA nuclease or nickase, wherein the “CRISPR polypeptide” is derived from a CRISPR system of any organism, which can be cloned and used for targeted genome engineering. The terms “CRISPR nuclease” or “CRISPR polypeptide” also comprise mutants or catalytically active fragments or fusions of a naturally occurring CRISPR effector sequence, or the respective sequences encoding the same. A “CRISPR nuclease” or “CRISPR polypeptide” may thus, for example, also refer to a CRISPR nickase or even a nuclease-deficient variant of a CRISPR polypeptide having endonucleolytic function in its natural environment.

The terms “guide RNA”, “gRNA”, “single guide RNA”, or “sgRNA” are used interchangeably herein and either refer to a synthetic fusion of a CRISPR RNA (crRNA) and a trans-activating crRNA (tracrRNA), or the term refers to a single RNA molecule consisting only of a crRNA and/or a tracrRNA, or the term refers to a gRNA individually comprising a crRNA or a tracrRNA moiety. A tracr and a crRNA moiety, if present as required by the respective CRISPR polypeptide, thus do not necessarily have to be present on one covalently attached RNA molecule, yet they can also be comprised by two individual RNA molecules, which can associate or can be associated by non-covalent or covalent interaction to provide a gRNA according to the present disclosure. In the case of single RNA-guided endonucleases like Cpf1 (see Zetsche et al., 2015, supra), for example, a crRNA as a single guide nucleic acid sequence might be sufficient for mediating DNA targeting.

The term “zinc finger nuclease,” as used herein, refers to a nuclease comprising a nucleic acid cleavage domain conjugated to a binding domain that comprises a zinc finger array. The cleavage domain may be the cleavage domain of the type II restriction endonuclease FokI. Zinc finger nucleases can be designed to target virtually any desired sequence in a given nucleic acid molecule for cleavage, and the possibility to the design zinc finger binding domains to bind unique sites in the context of complex genomes allows for targeted cleavage of a single genomic site in living cells. Targeting a double-strand break to a desired genomic locus can be used to introduce InDels into the nucleotide sequence of a desired genomic locus. Zinc finger nucleases can be generated to target a site of interest by methods well known to those of skill in the art. For example, zinc finger binding domains with a desired specificity can be designed by combining individual zinc finger motifs of known specificity. The structure of the zinc finger protein Zif268 bound to DNA has informed much of the work in this field and the concept of obtaining zinc fingers for each of the 64 possible base pair triplets and then mixing and matching these modular zinc fingers to design proteins with any desired sequence specificity has been described (Pavletich N P, Pabo C O (1991); “Zinc finger-DNA recognition: crystal structure of a Zif268-DNA complex at 2.1 A”. Science 252 (5007): 809-17).

The term “TAL effector nucleases” (TALENs) as used herein refer to sequence-specific nucleases or nucleic acids encoding the same. TAL effectors are proteins of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Thus, target sites can be predicted for TAL effectors, and TAL effectors can also be engineered and generated for the purpose of binding to particular nucleotide sequences. Specificity depends on an effector-variable number of imperfect, typically 34 amino acid repeats (Schornack et al. (2006) J. Plant Physiol. 163:256). Polymorphisms are primarily at repeat positions 12 and 13, which are referred to herein as the repeat variable-diresidue (RVD). RVDs of TAL effectors correspond to the nucleotides in their target sites in a direct, linear fashion, one RVD to one nucleotide, with some degeneracy and no apparent context dependence. This finding represents a valuable mechanism for protein-DNA recognition that enables target site prediction for new target specific TAL effector. TAL effectors per se do not comprise a nuclease domain. TAL effector nucleases or TALENs therefor represent fusion construct in which the TAL effector-encoding nucleic acid sequences is fused to a sequence encoding a nuclease or a portion of a nuclease, typically a nonspecific cleavage domain from a type II restriction endonuclease such as FokI (Kim et al. (1996) Proc. Natl. Acad. Sci. USA 93:1156-1160). Other useful endonucleases which can be fused to the effector domain may include, for example, HhaI, HindIII, Nod, BbvCI, EcoRI, BglI, and AlwI. The fact that some endonucleases (e.g., FokI) only function as dimers can be capitalized upon to enhance the target specificity of the TAL effector. For example, in some cases each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

As used herein, the term “meganuclease” refers to an endonuclease that binds double-stranded DNA at a recognition sequence that is greater than 12 base pairs. Naturally-occurring meganucleases can be monomeric (e.g., I-SceI) or dimeric (e.g., I-CreI). The term meganuclease, as used herein, can be used to refer to monomeric meganucleases, dimeric meganucleases, or to the monomers which associate to form a dimeric meganuclease. The term “homing endonuclease” is synonymous with the term “meganuclease. Due to the large recognition site of meganucleases, this site generally occurs only once in any given genome. Meganucleases can therefore be used to achieve very high levels of gene targeting efficiencies in mammalian cells and plants (Rouet et al., MoI. Cell. Biol., 1994, 14, 8096-106; Choulika et al., MoI. Cell. Biol., 1995, 15, 1968-73). Among meganucleases, the LAGLIDADG family of homing endonucleases has become a valuable tool for the study of genomes and over the past years. The term “LAGLIDADG meganuclease” refers either to meganucleases including a single LAGLIDADG motif, which are naturally dimeric, or to meganucleases including two LAGLIDADG motifs, which are naturally monomeric.

For example, the at least one InDel may also be introduced using a programable base editor, optionally in combination with a programable nuclease. The programable “base editor” as used herein refers to a protein or a fragment thereof having the same catalytical activity as the protein it is derived from, which protein or fragment thereof, alone or when provided as molecular complex, referred to as base editing complex herein, has the capacity to mediate a targeted base modification, i.e., the conversion of a base of interest resulting in a point mutation of interest. Preferably, the at least one base editor in the context of the present invention is temporarily or permanently linked to at least one site-specific, programable effector, or optionally to a component of at least one site-specific, programable effector complex. The linkage can be covalent and/or non-covalent. Multiple publications have shown targeted base conversion, primarily cytidine (C) to thymine (T), using a CRISPR/Cas9 nickase or non-functional nuclease linked to a cytidine deaminase domain, Apolipoprotein B mRNA-editing catalytic polypeptide (APOBEC1), e.g., APOBEC derived from rat. The deamination of cytosine (C) is catalysed by cytidine deaminases and results in uracil (U), which has the base-pairing properties of thymine (T). Most known cytidine deaminases operate on RNA, and the few examples that are known to accept DNA require single-stranded (ss) DNA. Studies on the dCas9-target DNA complex reveal that at least nine nucleotides (nt) of the displaced DNA strand are unpaired upon formation of the Cas9-guide RNA-DNA ‘R-loop’ complex (Jore et al., Nat. Struct. Mol. Biol., 18, 529-536 (2011)). Indeed, in the structure of the Cas9 R-loop complex, the first 11 nt of the protospacer on the displaced DNA strand are disordered, suggesting that their movement is not highly restricted. It has also been speculated that Cas9 nickase-induced mutations at cytosines in the non-template strand might arise from their accessibility by cellular cytosine deaminase enzymes. It was reasoned that a subset of this stretch of ssDNA in the R-loop might serve as an efficient substrate for a dCas9-tethered cytidine deaminase to effect direct, programmable conversion of C to U in DNA (Komor et al., supra). Recently, Goudelli et al ((2017). Programmable base editing of A•T to G•Cin genomic DNA without DNA cleavage. Nature, 551(7681), 464.) described adenine base editors (ABEs) that mediate the conversion of A•T to G•C in genomic DNA.

Any base editing complex according to the present invention can thus comprise at least one cytidine deaminase, or a catalytically active fragment thereof. The at least one base editing complex can comprise the cytidine deaminase, or a domain thereof in the form of a catalytically active fragment, as base editor.

In one embodiment of the present invention, a donor plant comprising a desired trait may be modified, for example, by using a programmable nuclease to introduce an InDel into a suitable genomic locus as described herein to generate an artificial InDel marker allele. In the case where the artificial InDel marker allele comprises a deletion, primers specific for the deleted sequence are designed. A person skilled in the art will readily be able to design suitable primers.

A “primer” as used herein refers to an oligonucleotide (synthetic or occurring naturally), which is capable of acting as a point of initiation of nucleic acid synthesis or replication along a complementary strand when placed under conditions in which synthesis of a complementary strand is catalysed by a polymerase. Typically, primers are about 10 to 30 nucleotides in length, but may be longer or shorter. Primers may be provided in double-stranded form, though the single-stranded form is more typically used. A primer can further contain a detectable label, for example a 5′ end label.

After crossing the donor plant with a wildtype plant, progeny samples are analyzed for the presence or absence of the deletion marker allele by, for example, (q)PCR, or other suitable techniques. “Wild type” plant/organism as defined herein is taken to mean an unmodified plant of the same species or variety as the donor plant into which the at least one InDel has been introduced.

Signals obtained for primers specific for the deleted sequence indicate that the progeny plant has the wildtype genotype, lacking the donor trait of interest or at least that the trait of interest was not inherited in a homozygous way by the progeny plant. Conversely, no signal for primers 1+2 (see FIG. 1A) could suggest homogenous multiplication of the donor trait. It however remains uncertain whether no signal is definitively due to the absence of the donor trait or whether other factors are responsible (e.g. insufficient primer annealing etc.). In order to increase primer specificity, the deletion is preferably at least approximately 10 bp in length, preferably approximately 20 bp in length.

Where the artificial InDel marker allele comprises a combination of insertions and deletions, insertions and deletions linked to the desired trait may be inserted using a programable nuclease, as defined herein, at a predetermined genomic locus within a flanking region of the nucleic acid of interest, which preferably encodes a polypeptide conferring a trait of interest. Primers specific for the insertion (see primers 3+4 in the illustration below) can be used for the detection of the donor trait. Since the insertion is absent in the wildtype, a positive signal reliably indicates the presence of the donor trait in the progeny plant making the assessment of the presence or absence of a desired trait highly specific and more accurate. Furthermore, primers specific for the deletion marker can be used for the identification of progeny plants which do contain the desired trait (no signal for primers 1+2).

The combination of an insertion with a deletion is thus more reliable when determining the presence or absence of a desired trait, since the presence/absence is determined by a positive PCR signal. This approach allows for the assessment whether a desired trait is present in the genome of progeny samples obtained from crossing a donor plant and a wild type plant and whether the donor trait was multiplied in a homozygous or heterozygous manner.

According to a preferred embodiment, the method of the invention therefore comprises introduction of an InDel comprising an insertion and a deletion. The insertion and/or deletion is preferably at least 10, 11, 12, 13, 14, 15, 16, 17, 18, 19 or 20 base pairs.

As illustrated in FIG. 1B, in such preferred embodiment, primers 1+2 may be located completely in the region of deletion so that only the wildtype genotype is detected. Even if one of the primers is located outside the deletion (or both primers partially), the marker system remains specific, since PCR products will only be obtained for the wildtype genotype. Specificity of the marker system is thus assured as long as most of the primer(s) (e.g. 10 bp) is located in the region of deletion.

In addition to primers 1+2 specific for the deletion, additional primers may be used which are specific for the insertion. For example, primers 3+4 may be located completely in the region of the insertion so that only the donor genotype is detected. Even if one of the primers is located outside the insertion (or both primers partially), the marker system remains specific, since PCR products will only be obtained for the donor genotype provided that at least one primer is located in the region of insertion.

The combination of an insertion and a deletion in the herein described methods of the invention and the use of primers specific for the inserted InDels thus provide a reliable strategy for determining the presence or absence of a desired trait, since the presence/absence is determined by a positive PCR signal which significantly reduces the chance of “false positive” or “false negatives”.

In a further embodiment of the herein described methods of the invention, the at least one InDel may advantageously be introduced into the genomic locus in the genome of a donor plant (comprising the nucleic acid of interest, preferably encoding a polypeptide conferring the trait of interest) at the beginning of the breeding process, i.e. before the donor is crossed with a desired elite line. An “elite line” means any line that has resulted from breeding and selection for superior agronomic performance. Numerous elite lines are available and known to those of skill in the art.

The abovementioned approach ensures that all elite lines which are crossed with the donor can be readily screened for the InDel marker allele. By designing a screening assay based on the InDel marker allele generated in the genomic background of a given donor, it is possible to use one established screening system for different elite lines to assess whether the desired trait has been inherited by such edited line. This approach avoids laborious and time-consuming development of marker assays designed for the genetic background of a given elite line into which the InDel marker allele has been inserted by crossing the elite line with the donor line. With the above described method, it is therefore possible to assess whether different elite lines contain the desired trait of the donor by applying one established screening method which was designed for the genomic background of the trait donor. Furthermore, side effects (e.g. pleiotropic effects) on phenotype due to the genome editing can be tested in parallel.

Furthermore, if InDel marker alleles are already used in a breeding process, one or several elite donors may be edited to generate a second donor generation suitable for the concept of marker-assisted breeding and quality control (see FIG. 1C).

FIG. 2 illustrates exemplary the above-mentioned breeding process. A homozygous donor comprising a desired trait (asterisk) is linked to an artificial InDel marker allele (grey filled). The InDel polymorphism has been introduced into a genomic locus of a suitable flanking region of a nucleic acid of interest associated with a desired trait (black filled) via a programmable nuclease. In a common breeding process, the homozygous donor is crossed with several elite lines to obtain (after backcrossing/selfing and selection) homozygous elite lines comprising the nucleic acid of interest associated with the desired trait. Due to the development of an InDel marker allele specific for the genomic background of the donor line, the elite lines can be screened for the InDel marker allele associated with the desired trait by using one single screening assay designed specifically for the flanking region of the InDel marker allele of the donor genotype. Based on this approach, there is thus no need to develop screening assays specific for the different genotypic flanking regions of the different elite lines. The insertion of the at least one InDel into a genomic locus genetically linked to the donor trait at the very beginning of the breeding process (introgression process) into the elite line therefore provides a method to screen different elite lines for the insertion of a desired trait independently of their respective genomic background.

The term “backcrossing” as referred to herein is a process in which a progeny plant is repeatedly crossed back to one of its parents. The “donor” comprises the nucleic acid sequence of interest associated with the desired trait linked to the InDel marker allele and which is to be introgressed into the recipient line. The “recipient” may be an elite line or any other plant into which the nucleic acid of interest is to be introgressed. “Introgression” as defined herein refers to the transmission of a desired allele of a genetic locus from one genetic background to another. The initial cross gives rise to the F1 generation. As shown in FIG. 2, a backcross is performed repeatedly across several generations (with a progeny individual of each successive backcross generation being itself backcrossed to the same parental genotype) until a homozygous elite line comprising the trait of interest linked to the InDel marker allele is obtained.

As used herein, “selecting” or “selection” in the context of marker-assisted selection or breeding refers to the act of picking or choosing desired individuals, normally from a population, based on certain pre-determined criteria. Suitable selection techniques are commonly known and are a routine part of an experimental setup for any skilled person in the field of plant breeding.

The InDel introduction into the genomic locus results in the creation of an artificial marker allele which is inheritable to subsequent generations of the organism along with the nucleic acid of interest. The artificial marker allele (the InDel once introduced into the genomic region) may be detectable and distinguishable on the basis of its polynucleotide length and/or sequence.

The artificial marker allele may therefore be detected using any available method for the detection of polymorphisms in genomic DNA samples, such detection tools and methods are referred to herein as “molecular markers”. The genomic DNA sample may be genomic DNA isolated directly from a plant, cloned genomic DNA, or amplified genomic DNA.

PCR-based methods are preferred for the detection of the artificial marker allele, however any of various hybridization techniques with specific probes including Southern blotting, in-situ hybridization and comparative genomic hybridization may alternatively be used. Furthermore, DNA digestion and high-solution capillary electrophoresis can be used to detect artificial marker alleles. Other suitable detection methods include microarrays, mass spectrometry-based methods, and/or nucleic acid sequencing methods.

In a preferred embodiment of the invention, the molecular marker is defined as a pair of primers specific for the artificial marker allele, i.e. the predetermined genomic locus comprising the at least one InDel, or the wild type genomic locus. The primers are preferably at least 10, 11, 12, 13, 14, 15, 16, 17, 18, 19 or 20 base pairs in length.

Marker assays for target genes are also mostly available. However, these assays are not always fully diagnostic/unique. The fully diagnostic marker allele is in every case the functional polymorphism. However, due to the characteristics of the flanking regions of the nucleic acid/trait of interest, it is not always possible to design suitable marker assays for marker-assisted selection. In addition, in case of a functional SNP marker allele, it is not possible to develop highly sensitive assays that would be suitable for reliable quality control assays for new traits in the breeding process. An InDel marker allele, like the one described herein, can be applied in marker-assisted selection of the target trait and would be applicable in highly sensitive quality control assays. For example, the inventive marker alleles can be used to assure purity of seed multiplications regarding the respective target trait and to avoid contaminations of seeds containing an undesired trait or which lack the desired trait of interest. Although, sensitive assays can in principle be developed based on SNP polymorphisms, the sensitivity of SNP detection is technically limited and significantly lower compared to the herein described artificial InDel marker alleles, since the detection of the polymorphism is based on only one single base pair mismatch, which can easily result in the detection of false positives or false negatives. In case of the InDel polymorphisms described herein it is possible to detect one (undesired) allele among several thousand samples, whereas a SNP polymorphism would allow detection of an (undesired) allele only within a few dozen samples.

According to a second aspect of the present invention, there is provided a method for determining the presence of a nucleic acid of interest, preferably encoding a polypeptide conferring a trait of interest, in a mixed population of individuals comprising the nucleic acid of interest and individuals not comprising the nucleic acid of interest, said method comprising detection of an artificial marker allele as defined in the first aspect of the invention using at least one molecular marker specific for the artificial marker allele and/or at least one molecular marker specific for the wild type genomic locus.

The at least one molecular marker is as defined herein in the first aspect of the invention and is preferably a pair of primers annealing to the wild type genomic locus or the artificial marker allele. Preferably the primers allow the detection of the artificial marker allele comprising an insertion and deletion marker. The primers may be specific to the inserted or deleted sequences in the genomic locus. The primers are preferably at least 10, 11, 12, 13, 14, 15, 16, 17, 18, 19 or 20 base pairs in length.

A “population” of plants means a set comprising any number of physical individuals or samples or data taken therefrom for evaluation.

According to a third aspect of the present invention, there is provided a method for assessing the homogeneity of a population of individuals comprising a nucleic acid of interest, preferably encoding a polypeptide conferring a trait of interest, said method comprising detection of an artificial marker allele as defined in the first aspect of the invention and determining homogeneity in the population by using at least one molecular marker specific for the artificial marker allele and/or at least one molecular marker specific for the wild type genomic locus, wherein the detection of the wild type genomic locus indicates heterogenous distribution of individuals comprising the nucleic acid of interest in the population.

Preferably the at least one molecular marker is a pair of primers annealing to the wild type genomic locus or the artificial marker allele. Preferably the primers allow the detection of the artificial marker allele comprising an insertion and deletion marker. The primers may be specific to the inserted or deleted sequences in the genomic locus. The primers are preferably at least 10, 11, 12, 13, 14, 15, 16, 17, 18, 19 or 20 base pairs in length.

According to a fourth aspect of the present invention, there is provided a method for introgressing a nucleic acid of interest, preferably encoding a polypeptide conferring a trait of interest, to a population of individuals, comprising the steps of:
(i) making an artificial marker allele according to the first aspect of the invention in a donor organism comprising the nucleic acid of interest;
(ii) crossing said donor organism with a recipient organism of the same species not comprising the nucleic acid of interest to generate progeny of heterogenous genetic composition;
(iii) backcrossing/selfing and selection for the presence of the artificial marker allele to obtain progeny of homozygous genetic composition, which comprise the nucleic acid of interest in the background of the recipient organism,
(iv) optionally, repeating step (iii) at least once, preferably several times.

Step (iii) of the method is based on detection using at least one molecular marker specific for detection of the presence of the artificial marker allele in the progeny and/or at least one molecular marker specific for detection of the absence of the artificial marker allele in the progeny. Preferably, the at least one molecular marker is a pair of primers annealing to the wild type genomic locus or the artificial marker allele. Preferably the primers allow the detection of the artificial marker allele comprising an insertion and deletion marker. The primers may be specific to the inserted or deleted sequences in the genomic locus. The primers are preferably at least 10, 11, 12, 13, 14, 15, 16, 17, 18, 19 or 20 base pairs in length.

The recipient organism may be an elite line, a wild type organism or a transgenic organism. The terms “wild type” and “elite” are as defined herein. The term “transgenic” refers to organisms into which a gene or genetic material has been transferred (typically by any of a number of genetic engineering techniques) from one organism to another or from the same organism but where the genetic material is not at its natural locus in the genome.

According to a fifth aspect of the present invention, there is provided a method for making an artificial marker allele specific for a nucleic acid of interest comprising designing one or more genotype-specific InDels and introducing said InDels into a genomic locus in the genome of an organism, wherein the genomic locus is linked to the nucleic acid of interest. The organism may comprise at least 1, 2, 3, 4, 5, 6, 7, 8, 9, 10, 11, 12, 13, 14, 15, 16, 17, 18, 19, 20 or more separate InDels, which create a fingerprint of sorts for detection and tracking purposes.

Also provided is an artificial marker allele comprising at least one genotype-specific InDel obtainable by the aforementioned method.

According to a sixth aspect of the present invention, there is provided use of an artificial marker allele according to the fifth aspect or use of an artificial marker allele obtainable by a method according to the first aspect of the present invention in marker assisted breeding.

A further aspect of the invention relates to the use of the InDel marker allele in combination with the modification of an endogenous gene of interest. Modification of a gene of interest can be achieved by commonly known gene editing approaches (e.g. site-directed nucleases, including CRISPR nuclease systems, Zinc-finger nucleases, TALENs, meganucleases and the like) to generate an “artificial trait” of interest. The combined use of GE based gene modification and the herein described artificial InDel marker alleles readily allow the direct and reliable detection of regenerated modified plants (from gene edited plant material) or modified progenies thereof.

Reference herein to an “endogenous” gene not only refers to the gene in question as found in a plant in its natural form (i.e., without there being any human intervention), but also refers to that same gene (or a substantially homologous nucleic acid/gene) in an isolated form subsequently (re)introduced into a plant (a transgene). For example, a transgenic plant containing such a transgene may encounter a substantial increase or reduction of the transgene expression and/or substantial increase or reduction of expression of the endogenous gene. The isolated gene may be isolated from an organism or may be manmade, for example by chemical synthesis.

Also provided herein is the use of a programmable nuclease for the generation of an artificial marker allele for the identification of a nucleic acid of interest in the genome of an organism. The programmable nuclease may be selected from CRISPR nuclease systems, zinc finger nucleases, TALENs, or meganucleases as described herein.

According to a seventh aspect of the present invention, there is provided a plant or seed comprising an artificial marker allele obtainable by a method according to the first aspect or comprising an artificial marker allele according to the fifth aspect of the present invention. The plant may be any plant and may for example be a plant selected from Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawing, in which:

FIG. 1A-C shows a schematic representation of marker-assisted analyses and a quality control assay in which the purity of multiplied seeds having a desired trait can be assured, by using the InDel approach of the present invention.

FIG. 2 shows a schematic representation of a breeding process in which an InDel is introduced into the genomic locus of a donor plant (comprising the nucleic acid of interest, preferably encoding a polypeptide conferring the trait of interest) at the beginning of the breeding process, i.e. before the donor is crossed with a desired elite line.

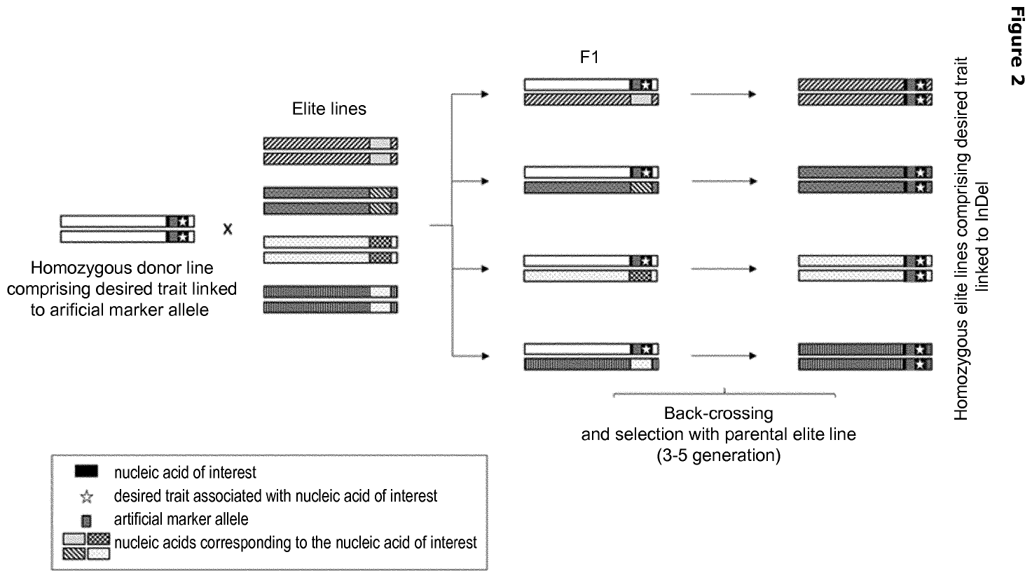

FIG. 3 shows InDel marker-assisted selection of a gene encoding a mutated cytochrome P450 oxidase conferring male sterility.

FIG. 4 shows InDel marker-assisted selection of a gene encoding a point-mutated acetolactate synthase conferring herbicide resistance.

### EXAMPLES

### Example 1: Deletion Marker Allele

### Introduction of a Deletion by Genome Editing for Marker-Assisted Selection

This example demonstrates the use of a deletion marker for the detection of a desired trait, which would be otherwise difficult to identify due to the characteristics of the genomic regions flanking the causative polymorphism.

The Beta vulgaris mutant BvCYP703A2_gst as disclosed in DE 10 2016 106 656.7 comprises a deletion in the gene encoding for a cytochrome P450 oxidase which confers to the mutant a male sterile phenotype (wildtype (WT) BvCYP703=BvCYP703_WT (SEQ ID NO: 75)). This phenotype can be used e.g. to improve breeding programs and for the production of hybrid seeds.

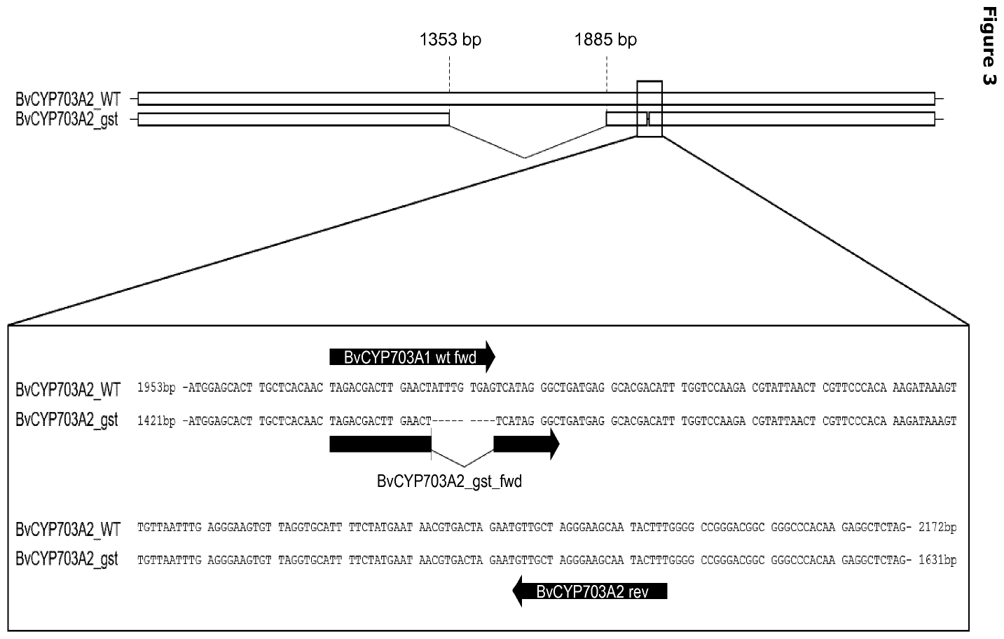

The mutant BvCYP703A2_gst (SEQ ID NO: 76) comprises a large deletion between position 1560 and 2100 (see FIG. 3). However, due to the characteristics of the genomic regions flanking the deletion (i.e. highly repetitive and high AT content) it is difficult (if not impossible) to design suitable primers and assays that would allow direct detection of the causative polymorphism itself. It would therefore not be possible to screen progeny plants, which were obtained by crossing a wild-type plant with the donor, for the desired genotype by direct detection of the deletion due to the lack of suitable detection assays.

The inventors have therefore identified a region in the flanking region of the gene encoding a cytochrome P450 oxidase which is suitable for InDel marker-assisted selection of the desired genotype. The naturally occurring deletion causing the trait (male sterility) is located between positions −200 and +333 of the BvCYP703A2 gene (numbering starts at the translation initiation site). Since the deletion causes a disruption of the gene, there is no doubt that remaining gene features (e.g. exons) are unfunctional and additional manipulation within the remaining exons does not cause pleiotropic effects. Therefore, parts of remaining exon 1, spanning region +334 to +500 were chosen as target site for an artificial InDel marker allele. The maximum distance from the deletion position +334 to the end of the region of interest (+500) is 166 bp corresponding to a genetic distance of 0.00096 cM. Blast analysis of the 166 bp fragment did not reveal unspecific hits in the sugar beet genome. Further sequence analysis (repetitivity, GC content, base distribution) led to definition of region +434 to +443 as target site for an artificial deletion, with an InDel specific primer set between positions +420 to +449.

A deletion is inserted into this target site via genomic editing as described herein (SEQ ID NO: 77). Suitable primers are designed specific to the flanking region of the deletion marker (see above). Due to its tight linkage to the desired genotype, this deletion can then be used to identify progeny plants conferring male sterility. For homo/heterogenous detection of the deletion two PCR reactions should be performed.

Possible primers which can be used for the detection of the donor and/or wild type strain may be:

BvCYP703A2_WT_fwd:
(SEQ ID NO: 45)
5′-TAGACGACTTGAACTATTTGTGAG-3′
BvCYP703A2_gst_fwd:
(SEQ ID NO: 46)
5′-TAGACGACTTGAACTTCATAGGGC-3′
BvCYP703A2_rev:
(SEQ ID NO: 47)
5′-AAAGTATTGCTTCCCTAGCAACA-3′

### Example 2: Insertion Marker Allele

### Introduction of an Insertion by Genome Editing for Marker-Assisted Selection

This example demonstrates that a desired trait, which is difficult to detect because its causal link is a single nucleotide polymorphism (SNP), can be reliably identified by using the herein described InDel marker approach.

In this example, a single point mutation at position +1706 in the gene encoding for the enzyme acetolactate synthase confers resistance to sulfonyl urea herbicides in a Beta vulgaris plant (as disclosed in WO 2012/049268; wildtype (WT) BvALS=BvALS_WT (SEQ ID NO: 78; point-mutated BvALS=BvALS_SU_res (SEQ ID NO: 79)). This single nucleotide polymorphism is difficult to detect because primers designed specifically for screening plants having the donor trait would differ in only one single nucleotide in comparison to the wild-type sequence, thereby increasing the likelihood of false-positives and/or false negatives which limits the quality of the screen.

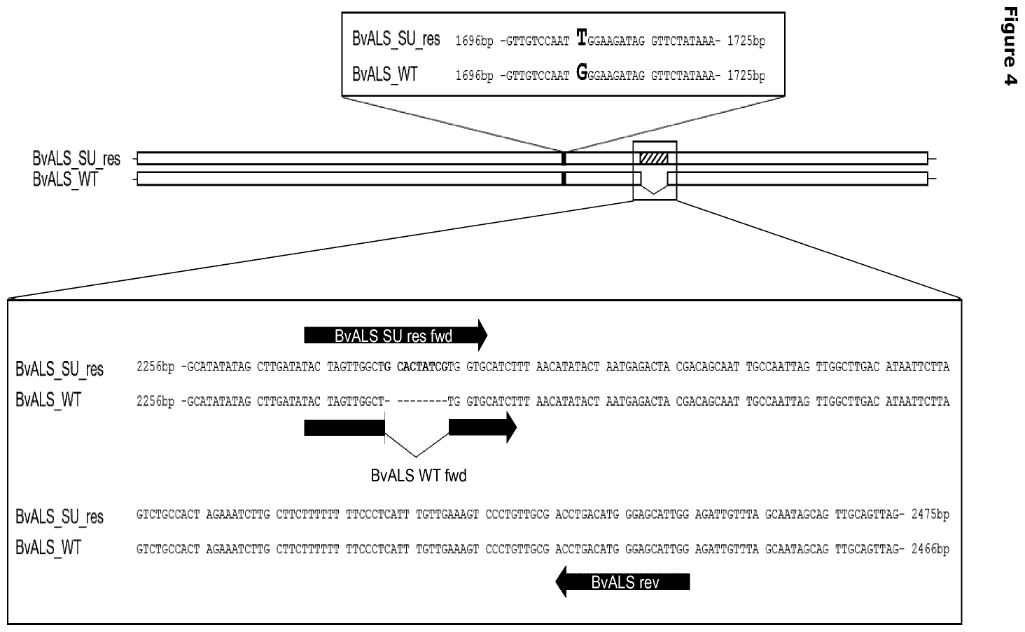

This drawback can be overcome by introducing an InDel marker into the flanking region of the mutated gene encoding for acetolactate synthase (see FIG. 4).

The inventors have identified a morphogenic flanking region of the mutated gene suitable for the design of an artificial marker allele. The SNP causing the trait (SU resistance, W569L) is located at position +1706 of the BvALS gene (numbering starts at the translation initiation site). The annotated 3′UTR region of the gene ends at position +2252. The inventors were unable to localize a genomic feature starting from position +2253 to +4000. The maximum distance from the SNP position +1706 to the end of the region of interest (+4000) is 2294 bp corresponding to a genetic distance of 0.00036 cM. Blast analysis of the 2294 bp fragment did not reveal unspecific hits in the sugar beet genome. Iterative sequence analysis (blast, alignments) led to selection of region +2274 to +2445 suitable for artificial InDel placement. Further sequence analysis (repetitively, GC content, base distribution) led to definition of region +2285 to +2293 as artificial target site, with an InDel specific primer set between positions +2274 to +2303 (see FIG. 4).

Into this target site a 9 bp long insertion can be inserted which is non-homologous and unique to the genomic pool of the donor line (SEQ ID NO: 80). Suitable primers are designed for the flanking regions of the insertion as described herein. For homo/heterogenous detection of the insertion marker two PCR reactions are required.

Based on this approach it is then possible to screen progeny plants, obtained from crossing the donor with the wild-type plant, for the insertion of the desired mutation conferring herbicide resistance without the need to rely on the causative polymorphism itself.

Possible primers which can be used for the detection of the donor and/or wild type strain may be:

BvALS_WT_fwd:
(SEQ ID NO: 48)
5′-ACTAGTTGGCTTGGTGCATCT-3′
BvALS_SU_res_fwd:
(SEQ ID NO: 49)
5′-ACTAGTTGGCTGCACTATCGTGC-3′
BvALS_rev:
(SEQ ID NO: 50)
5′-CCAATGCTCCCATGTCAGGT-3′

### Example 3: Quality Control Assay to Assure Purity of Seed Multiplications for a Respective Trait

This example illustrates how purity of multiplied seeds having a desired trait can be assured by using the herein described InDel approach.

In this example, the donor line comprising a desired trait is modified by introducing a nucleotide sequence (GCACTATCG) into its genome to generate an artificial insertion marker allele which is tightly linked to the desired trait.

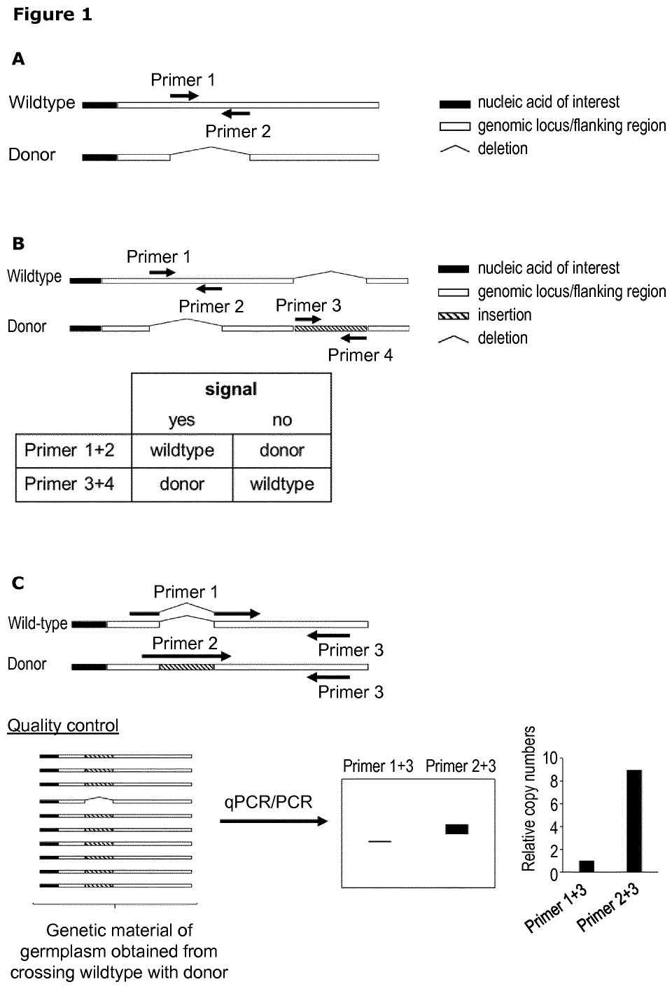

After crossing the donor comprising the insertion marker allele with a wildtype plant, which does not contain the artificial marker allele, F1 progeny plants are obtained which are heterogenous in their genetic composition. Backcrossing and subsequent selection result in plants which contain the trait of interest within the genetic background of the wildtype plant. In order to ensure homogeneity and purity of seed multiplication of plants comprising the desired trait, seed samples are analyzed by using primer pairs specific for the wildtype (primers 1+3) and/or the donor (primer 2+3). Analysis of the seed samples by e.g. (q)PCR then readily allows assessment of the degree of purity (see FIG. 1C).

Based on this quality control assay, it is thus possible to reliably assess whether the tested seed samples are homozygous for the desired trait or whether the seeds are “contaminated” with the wildtype gene/trait corresponding to the desired donor trait. Such quality control would not be possible, if the polymorphism linked to the desired trait is a single nucleotide polymorphism, since a single nucleotide mismatch does not offer sufficient resolution and specificity to ensure a reliable quality assessment by (q)PCR.

### Example 4: GE Based Technology for the Generation of Artificial InDel Marker Alleles which are Linked to a Desired Trait

This example provides a technical description on how to
(a) generate a deletion marker allele via GE based gene modification into a donor genome having a large deletion in the gene encoding a cytochrome P450 oxidase causing male sterility (Example 1),
(b) generate an insertion marker allele via GE based gene modification into a donor genome having a point mutation in the gene encoding for the enzyme acetate lactate synthase conferring herbicide resistance in a Beta vulgaris plant (Example 2), and
(c) modify an endogenous gene encoding the enzyme acetate lactate synthase by introducing a specific point mutation (G→T) via GE, thereby conferring herbicide resistance in a Beta vulgaris plant and generating an insertion marker allele linked to the artificially generated trait of interest.
   Design and Selection of crRNA:

Suitable crRNAs for Cpf1-induced induction of double strand breaks were designed by using the CRISPR RGEN Tools (http://www.rgenome.net/cas-designer/ [Park J., Bae S., and Kim J.-S. Cas-Designer: A web-based tool for choice of CRISPR-Cas9 target sites. Bioinformatics 31, 4014-4016 (2015). and Bae S., Park J., and Kim J.-S. Cas-OFFinder: A fast and versatile algorithm that searches for potential off-target sites of Cas9 RNA-guided endonucleases. Bioinformatics 30, 1473-1475 (2014).). Therefore, suitable protospacers within the genomic DNA sequence were identified and selected. To ensure functionality of Cpf1 endonuclease from Lachnospiraceae bacterium ND2006 (Lb) (SEQ ID NO: 51), protospacers with a length of 24 nucleotides were selected, wherein their genomic binding sequence at the 5′ end was flanked with an essential protospacer adjacent motif (PAM) having the sequence 5′-TTTV-3′ (V is G, C or A). Suitable protospacers were selected based on the prescribed quality criteria of the tool and analyzed for potential off-targets with an internal reference genome of B. vulgaris.

For further experiments crRNAs were selected, which in addition to the actual target sequence had at most 15 identical bases with a functional PAM. Since the first 18 nucleotides of the protospacer are essential for recognizing and cleaving the target sequence, it was thereby possible to avoid unwanted cleavage within other genomic sequences [Tang, X., L. G. Lowder, T. Zhang, A. A. Malzahn, X. Zheng, D. F. Voytas, Z. Zhong, Y. Chen, Q. Ren, Q. Li, E. R. Kirkland, Y. Zhang and Y. Qi (2017). “A CRISPR-Cpf1 system for efficient genome editing and transcriptional repression in plants.” Nat Plants 3: 17018.]. Based on this approach, the following potential crRNAs specific for various positions were identified (see Table A).

TABLE A
Selected target sequences.
PAM sequences are underlined.

| | Genomic target | | |
|---|---|---|---|
| | sequence | | |
| | with 5′- | Binding | |
| Name of | flanking PAM | on +/− | |
| crRNA | (underlined) | strand | Function |
| crRNA_ | TTTAggtatggttg | − | generating |
| ALS_G/T | tccaatgggaagat | | G→T point |
| | (SEQ ID NO: 1) | | mutation |
| | | | in ALS |
| crRNA_ | TTTCggctatggat | − | candidate 1 |
| ALS_In1 | gttgtgttgcatca | | for |
| | (SEQ ID NO: 2) | | generating |
| | | | an insertion |
| | | | marker for |
| | | | MAS in |
| | | | ALS |
| crRNA_ | TTTCtacgtggttc | + | Candidate 2 |
| ALS_In2 | ggttgatcatatag | | for |
| | (SEQ ID NO: 3) | | generating |
| | | | an |
| | | | insertion |
| | | | for MAS |
| | | | in ALS |
| crRNA_ | TTTGtacgtggttcg | + | Generating a |
| CYP_Del | gttgatcatatag | | deletion |
| | (SEQ ID NO: 4) | | marker |
| | | | for |
| | | | MAS in |
| | | | CYP |

### Cloning of Genetic Elements:

For the cloning of Cpf1- and crRNA expression cassettes, a hindering recognition sequence of the restriction enzyme BbsI was removed from the target vector pZFNnptII by introducing a point mutation (T→G). The mutagenesis was performed with a commercially available mutagenesis kit according to the manufacturer's instructions by using two mutagenesis primers (see Table B).

TABLE B
Mutagenesis primers used for the
introduction of the point mutation
(G→T, underlined) for removal of the
BbsI recognition sequence

| Name | Sequence 5′→3′ |
|---|---|
| Mutagenesis | CAGTGCAGCCGTCG |
| primer 1 | TCTGAAAACGACA |
| | (SEQ ID NO: 5) |
| Mutagenesis | AACGTCAGAAGCC |
| primer 2 | GACTGCACTATAG |
| | (SEQ ID NO: 6) |

For the expression of the Lbcpf1 gene in B. vulgaris a DNA fragment comprising a DNA sequence, codon-optimized for A. thaliana, was synthesized wherein the DNA sequence had a 5′ flanking PcUbi promoter sequence from Petroselinum crispum and a 3′ flanking 3A terminator sequence from Pea sp. (SEQ ID NO: 52). Restriction cleavage sites within the coding sequence of Lbcpf1 which are relevant for cloning, were removed by introducing silent mutations (i.e. nucleotide exchange without effecting the amino acid sequence). Codon-optimization was performed based on the GeneArt algorithm from ThermoScientific. To allow the transport of cpf1 into the nucleus of the cell, the coding sequence of cpf1 was linked to a nuclear localization signal (NLS) from SV40 at the 5′ end and a NLS from Nucleoplasmin at the 3′ end. For the ligation with the binary target vector pZFNnptII the expression cassette was flanked by two HindIII restriction cleavage sites. For the cloning of the crRNA-expression cassette an additional PstI cleavage site was inserted between the 5′ flanking HindIII cleavage site and the PcUbi promoter sequence. Ligation of pZFNnptII_LbCpf1 was done by following a standard protocol. Successful insertion of the PcUbi::Cpf1::TPea expression cassette (SEQ ID NO: 52) was confirmed via sequencing, wherein the used primers were designed to specifically bind to a region spanning the flanking region of the vector as well as parts of the expression cassette (see Table C).

TABLE C
Primers used for sequencing of the
PcUbi::Cpf1::TPea expression cassette
integrated into pZFNnptII vector

| Name | Sequence 5′→3′ |
|---|---|
| pSeq_LbCpf1_F1 | AGCGCAACGCAATTAATGTG |
| | (SEQ ID NO: 7) |
| pSeq_LbCpf1_R1 | GATGAAGCTGAGGTAGTACC |
| | (SEQ ID NO:8) |
| pSeq_LbCpf1_F2 | AGGAAGGTTAGCAAGCTCGAG |
| | (SEQ ID NO: 9) |
| pSeq_LbCpf1_R2 | TCTCGTCGACCTTCTGGATG |
| | (SEQ ID NO: 10) |
| pSeq_LbCpf1_F3 | ATGCTGAGTACGATGACATCC |
| | (SEQ ID NO: 11) |
| pSeq_LbCpf1_R3 | TAGACCTGCTTCTCAACCTTCA |
| | (SEQ ID NO: 12) |
| pSeq_LbCpf1_F4 | ACCACTCACTCCTCGATAAG |
| | (SEQ ID NO: 13) |
| pSeq_LbCpf1_R4 | AACGACAATCTGATCGGGTAC |
| | (SEQ ID NO: 14) |

After transcription in a plant cell, crRNAs were intended to be cleaved by two flanking ribozymes. Therefore, the precursor crRNAs were flanked by the coding sequences of a Hammerhead ribozyme (SEQ ID NO: 53) and a HDV ribozyme (SEQ ID NO: 54) [Tang, X., L. G. Lowder, T. Zhang, A. A. Malzahn, X. Zheng, D. F. Voytas, Z. Zhong, Y. Chen, Q. Ren, Q. Li, E. R. Kirkland, Y. Zhang and Y. Qi (2017). “A CRISPR-Cpf1 system for efficient genome editing and transcriptional repression in plants.” Nat Plants 3: 17018.]. Other approaches exist for the transcription of crRNA, e.g. via PolII promoters, Cpf1 cleavage from mRNA, other ribozymes etc. For a seamless ligation of the single protospacer to the sequence of the crRNA repeats, two BbsI recognition sequences were integrated between the crRNA repeat and the HDV ribozyme, wherein the overhangs used for cloning were adjusted accordingly.

To ensure an identical expression strength of cpf1 and crRNAs, the crRNA ribozyme cassette was flanked by a PcUbi promoter sequence at the 5′ end and a 3A terminator sequence at the 3′ end. The crRNA expression cassette was flanked by two PstI cleavage sites for the later ligation into the pZFNnptII_Cpf1 target vector (SEQ ID NO: 55). The crRNA expression cassette (SEQ ID NO: 56) was commercially obtained as a synthetic DNA fragment. Ligation was performed by following a standard protocol. The correct insertion of the expression cassette was confirmed by multiple rounds of sequencing. The protospacer were ordered as complementary oligonucleotides and annealed according to standard protocols. The 24 bp long DNA fragments generated in this way were flanked by 4nt overhangs relevant for the ligation step (see Table D).

TABLE D
Sequences of oligonucleotides used for
the generation of 24 bp short protospacer.
4 nt overhangs used for ligation
are underlined

| Name crRNA | Sequence 5′→3′ |
|---|---|
| crRNA_ALS_G/T | AGATGGTATGGTTGTCCAATGGGAAGAT |
| | (SEQ ID NO: 15) |
| | GGCCATCTTCCCATTGGACAACCATACC |
| | (SEQ ID NO: 16) |
| crRNA_ALS_In1 | AGATGGCTATGGATGTTGTGTTGCATCA |
| | (SEQ ID NO: 17) |
| | GGCCTGATGCAACACAACATCCATAGCC |
| | (SEQ ID NO: 18) |
| crRNA_ALS_In2 | AGATTACGTGGTTCGGTTGATCATATAG |
| | (SEQ ID NO: 19) |
| | GGCCCTATATGATCAACCGAACCACGTA |
| | (SEQ ID NO: 20) |
| crRNA_CYP_Del | AGATTACGTGGTTCGGTTGATCATATAG |
| | (SEQ ID NO: 21) |
| | GGCCCTATATGATCAACCGAACCACGTA |
| | (SEQ ID NO: 22) |

The efficiency of the 4 crRNAs were tested via Agrobacterium induced gene transfer in leaves of B. vulgaris. The pZFNtDTnptII plasmid (SEQ ID NO: 57) was co-transformed to verify the transformation efficiency. Transformation of the leaf explants were done by vacuum infiltration following a standard protocol. The fluorescence of tDT was measured after six days by fluorescence microscopy. Explants with a heterogenous fluorescence were discarded. Leaf explants were shock-frozen in liquid nitrogen ten days after infiltration, ground and genomic DNA was isolated via the CTAB protocol. The efficiency of the single crRNAs was validated via NGS (external service provider) based on the number of inserted edits (e.g. number of insertions, deletions or nucleotide exchanges) relative to non-edited sequences in the genomic DNA.

Since all tested crRNAs showed activity, the crRNAs crRNA_ALS_G/T (SEQ ID NO: 58), crRNA_CYP_Del (SEQ ID NO: 59), crRNA_ALS_In1 (SEQ ID NO: 60) (most efficient) and crRNA_ALS_In2 (SEQ ID NO: 61), with the above described ribozyme, promoter and terminator sequences as reverse-oriented expression cassettes were ordered as synthetic DNA constructs (in total 4 constructs; for each crRNAs one construct (SEQ ID NO: 62, 63, 64, 65)). The DNA constructs were each flanked by two PstI restriction cleavage sites for cloning into the target vector pZFNnptII_LbCpf1 (SEQ ID NO: 55). After insertion of crRNAs, LbCpf1 and crRNA expression cassettes were ligated via HindIII from the pZFNnptII_LbCpf1_crRNA vector (SEQ ID NO: 23, 71, 72, 73, 74) into the pUbitDTnptII vector (SEQ ID NO: 66, 67, 68, 69, 70).

### Generation and Use of Repair Templates for HD-Repair

### ALS G→T Mutation

In order to generate the G→T point mutation, the repair template was designed to comprise 1000 bp upstream and downstream of the point mutation. The whole DNA template was ordered as a 2001 bp long synthetic DNA fragment (SEQ ID NO: 24) and directly used for transformation in the vector backbone of the provider. The repair template plasmid and the pUbitDTnptII_LbCpf1_crRNA plasmid (SEQ ID NO: 67) were introduced into B. vulgaris callus culture via biolistic co-bombardment using a gene gun according to an optimized delivery protocol. The transformation efficiency was validated based on the transient tDT fluorescence via fluorescence microscopy one day after transformation. The callus culture was cultivated in shoot induction medium in the absence of selective pressure (i.e. without Kanamycin). The regenerated shoots were subsequently tested for the site-directed mutation (in principle, if point mutation results in increased ALS resistance, such increase can be used for selection of the desired event). Therefore, genomic DNA was isolated via CTAB. Point mutations were amplified via two PCRs and the use of primers 5′ALS_G/T and ALS_G/T_Rv, as well as ALS_G/T_Fw and 3′ALS_G/T. Afterwards, PCR products were sequenced in each case with both primers. Here, it is important that binding of the first primer occurs within the homology region of the repair template and binding of the second primer outside of the 5′ and 3′ flanking homology regions of the repair template (see Table E).

TABLE E
Primers used for the detection of
point mutations

| | | Size of | |
|---|---|---|---|
| | Sequence | PCR | |
| Name | 5′→3′ | product | Binding |
| 5′ALS_G/T | GTT TTG GAT | 1138 bp | 5′ outside |
| | GTA GAG GAT | | the repair |
| | ATT CCT AGA | | template |
| | (SEQ ID NO: 25) | | |
| ALS_G/T_Rv | CAG GGA AGA | | Within the |
| | TAT CAG CAG | | repair |
| | ATT TG | | template |
| | (SEQ ID NO: 26) | | |
| ALS_G/T_Fw | CTA CAA TTA | 1127 bp | Within the |
| | GGG TGG AAA | | repair |
| | ATC TC | | template |
| | (SEQ ID NO: 27) | | |
| 3′ALS_G/T | CTC TAG TGG | | 3′ outside |
| | TCA CCT GGC | | the repair |
| | ATC | | template |
| | (SEQ ID NO: 28) | | |

In addition to the detection of the successful point mutation in the genome of B. vulgaris, the undesired integration of plasmid DNA was also analyzed. Therefore, genomic DNA, for which the successful integration of a point mutation at the desired locus had been confirmed, was analyzed for the presence of plasmid DNA via PCR. Sequence regions within the cpf1, the crRNA ribozyme cassette and the tDT were amplified using the primers listed in Table F below.

TABLE F
Primers used for the detection of sTABLE
integrated plasmid-specific sequences in
the genome of B. vulgaris shoots

| | | Size | |
|---|---|---|---|
| | Sequence | of PCR | |
| Name | 5′→3′ | product | Binding |
| pSEQ_ | ACCACTCACTCCTCGATAAG | 214 | Cpf1 |
| LbCpf1_F4 | (SEQ ID NO: 29) | | |
| pSeq_ | TAGACCTGCTTCTCAACCTT | | |
| LbCpf1_R3 | CA | | |
| | (SEQ ID NO: 30) | | |
| pSeq_ | TGCAGCGGATCCAAATTAC | 172 | crRNA- |
| Ribozyme_F | TG | | ribozyme |
| | (SEQ ID NO: 31) | | cassette |
| pSeq_ | CCTGGTCCCATTCGCCAT | | |
| Ribozyme_R | (SEQ ID NO: 32) | | |
| pSeq_ | TTACAAGAAGCTGTCCTTCC | 400 | tDT |
| tDT_F | (SEQ ID NO: 33) | | |
| pSeq_ | GTACTGTTCCACGATGGTGT | | |
| tDT_R | (SEQ ID NO: 34) | | |

### ALS 9 bp Insertion:

For the ALS 9 bp insertion an analogous approach was used as described for ALS G→T mutations above. The 9 bp insertion GCACTATCG was flanked upstream and downstream with a 1000 bp homologous sequence (SEQ ID NO: 35).

TABLE G
Primers used for the detection of the insertion

| | | Size of | |
|---|---|---|---|
| | sequence | the PCR | |
| Name | 5′→3′ | product | Binding |
| 5′ALS_ | GTG CTG ATG | 1148 + | 5′ outside |
| Insertion | TTA AAT TGG | 9 bp | the repair |
| | CAT TGC | | template |
| | (SEQ ID NO: 36) | | |
| ALS_ | CTA GTG GCA | | Within the |
| Insertion_ | GAC TAA GAA | | repair |
| Rv | TTA TG | | template |
| | (SEQ ID NO: 37) | | |
| ALS_ | GAA TGC TCT | 1165 + | Within the |
| Insertion_ | TCC TGT ATT | 9 bp | repair |
| Fw | GCT TG | | template |
| | (SEQ ID NO: 38) | | |
| 3′ALS_ | CAG TTC AAC | | 3′ outside |
| Insertion | ACA AAA GAA | | the repair |
| | GTT GTC | | template |
| | (SEQ ID NO: 39) | | |

### Combined Insertion of the G→T Point Mutation and the 9 bp Insertion in ALS:

In general, an analogous procedure is applied as for both approaches described above. In this case, however, the repair template is only flanked by 250 bp homologous sequences upstream and downstream, since homologues flanking sequences with 1000 bp upstream and downstream of the respective repair templates would overlap. In this setup, the plasmids for crRNA_ALS_G/T and crRNA_ALS_In1, as well as both repair templates were transformed using biolistic co-bombardment. Detection of the point mutation and the 9 bp insertion was done as described above.

### CYP Deletion Marker:

The deletion can also be generated and detected using one of the above described approaches. For the generation of a deletion marker, it is important that the repair template must contain the 9 bp deletion (ATTTGTGAG). This is then also flanked 1000 bp homologous sequences upstream and downstream of the repair template and used for the construct (SEQ ID NO: 40).

TABLE H
Primers used for the detection of the deletion

| | | Size of | |
|---|---|---|---|
| | Sequences | the PCR | |
| Name | 5′→3′ | product | Binding |
| 5′CYP_ | GTC TTT ACA | 1167 − | 5′ outside |
| Deletion | TAG CAA AAC | 9 bp | the repair |
| | AAT ATT GAA | | template |
| | G | | |
| | (SEQ ID | | |
| | NO: 41) | | |
| CYP_ | CTA ACA CTT | | Within the |
| Deletion_Rv | CCC TCA AAT | | repair |
| | TAA CAA C | | template |
| | (SEQ ID | | |
| | NO: 42) | | |
| CYP_ | CAA TAG TGG | 1198 − | Within the |
| Deletion_Fw | TGA TGT GGC | 9 bp | repair |
| | CTT GG | | template |
| | (SEQ ID | | |
| | NO: 43) | | |
| 3′CYP_ | GGT AAC TAG | | 3′ outside |
| Deletion | TAA AAG TAT | | the repair |
| | ACT CAT C | | template |
| | (SEQ ID | | |
| | NO: 44) | | |

1. A method for making an artificial marker allele for the identification of a nucleic acid of interest, preferably encoding a polypeptide conferring a trait of interest, in an organism, said method comprising:
(a) identifying at least one genomic locus in the genome of said organism, which is genetically linked to said nucleic acid of interest, and
(b) introducing at least one InDel into said at least one genomic locus,
thereby making a marker allele which is inheritable to subsequent generations of said organism along with said nucleic acid of interest.

2. The method according to claim 1, wherein said at least one InDel comprises at least one nucleotide insertion and/or at least one nucleotide deletion.

3. The method according to claim 1, wherein said genomic locus is unique within the genome of said organism and highly conserved across different genotypes of said organism and/or wherein the nucleotide sequence of the genomic locus obtained after insertion of the at least one artificial InDel is unique within the genome of said organism.

4. The method according to claim 1, wherein said genomic locus is positioned outside of any coding region, splicing signal or regulatory element of the nucleic acid of interest and/or is positioned in a region flanking the nucleic acid of interest or within the nucleic acid of interest.

5. The method according to claim 4, wherein the region flanking the nucleic acid of interest is located at the 3′ end of the nucleic acid of interest.

6. The method according to claim 5, wherein the region flanking the nucleic acid of interest is a distance of at least 2 cM or 1 cM or 0.5 cM or 0.1 cM from said nucleic acid of interest.

7. The method according to claim 1, wherein said at least one InDel comprises an insertion and wherein said insertion comprises a nucleotide sequence in the range of between 1 and 60 contiguous base pairs and which sequence is non-homologous to the genome of the organism in which said at least one InDel is introduced, preferably said insertion comprises a nucleotide sequence of at least 10 or at least 20 contiguous base pairs.

8. The method according to claim 1, wherein said at least one InDel comprises a deletion and wherein said deletion is in the range of between 1 and 60 contiguous base pairs, preferably at least 10 or at least 20 contiguous base pairs, relative to the corresponding wild type sequence of the genomic locus in which said at least one InDel is introduced.

9. The method according to claim 1, wherein said at least one InDel is introduced by a programmable nuclease, preferably said programmable nuclease is selected from CRISPR nuclease and guide RNA systems, zinc finger nucleases, TALENs, or meganucleases.

10. The method according to claim 1, wherein said nucleic acid of interest may be an endogenous gene, a heterologous gene, a mutated gene, a transgenic gene or a modified gene introduced or generated by gene editing or base editing.

11. A method for determining the presence of a nucleic acid of interest, preferably encoding a polypeptide conferring a trait of interest, in a mixed population of individuals comprising the nucleic acid of interest and individuals not comprising the nucleic acid of interest, said method comprising detection of an artificial marker allele as defined in claim 1 using at least one molecular marker specific for the artificial marker allele and/or at least one molecular marker specific for the wild type genomic locus.

12. A method for assessing the homogeneity of a population of individuals comprising a nucleic acid of interest, preferably encoding a polypeptide conferring a trait of interest, said method comprising detection of an artificial marker allele as defined in claim 1 and determining homogeneity in the population by using at least one molecular marker specific for the artificial marker allele and/or at least one molecular marker specific for the wild type genomic locus, wherein the detection of the wild type genomic locus indicates heterogenous distribution of individuals comprising the nucleic acid of interest in the population.

13. A method for making an artificial marker allele for the detection of a nucleic acid of interest comprising designing one or more genotype-specific InDels and introducing said InDels into a genomic locus in the genome of an organism, wherein the genomic locus is genetically linked to the nucleic acid of interest.

14. A method for utilizing an artificial marker allele obtainable by claim 1 in marker assisted selection.

15. A plant or seed comprising an artificial marker allele obtainable by a method according to claim 1.


SEQUENCE LISTING (80 sequences)

SEQ ID NO: 1 - DNA, 28 bp, Artificial Sequence
  crRNA_ALS_G/T
tttaggtatggttgtccaatgggaagat

SEQ ID NO: 2 - DNA, 28 bp, Artificial Sequence
  crRNA_ALS_In1
tttcggctatggatgttgtgttgcatca

SEQ ID NO: 3 - DNA, 28 bp, Artificial Sequence
  crRNA_ALS_In2
tttctacgtggttcggttgatcatatag

SEQ ID NO: 4 - DNA, 28 bp, Artificial Sequence
  crRNA_CYP_Del
tttgtacgtggttcggttgatcatatag

SEQ ID NO: 5 - DNA, 27 bp, Artificial Sequence
  Mutagenesis primer 1
cagtgcagccgtcgtctgaaaacgaca

SEQ ID NO: 6 - DNA, 26 bp, Artificial Sequence
  Mutagenesis primer 2
aacgtcagaagccgactgcactatag

SEQ ID NO: 7 - DNA, 20 bp, Artificial Sequence
  pSeq_LbCpf1_F1
agcgcaacgcaattaatgtg

SEQ ID NO: 8 - DNA, 20 bp, Artificial Sequence
  pSeq_LbCpf1_R1
gatgaagctgaggtagtacc

SEQ ID NO: 9 - DNA, 21 bp, Artificial Sequence
  pSeq_LbCpf1_F2
aggaaggttagcaagctcgag

SEQ ID NO: 10 - DNA, 20 bp, Artificial Sequence
  pSeq_LbCpf1_R2
tctcgtcgaccttctggatg

SEQ ID NO: 11 - DNA, 21 bp, Artificial Sequence
  pSeq_LbCpf1_F3
atgctgagtacgatgacatcc

SEQ ID NO: 12 - DNA, 22 bp, Artificial Sequence
  pSeq_LbCpf1_R3
tagacctgcttctcaaccttca

SEQ ID NO: 13 - DNA, 20 bp, Artificial Sequence
  pSeq_LbCpf1_F4
accactcactcctcgataag

SEQ ID NO: 14 - DNA, 21 bp, Artificial Sequence
  pSeq_LbCpf1_R4
aacgacaatctgatcgggtac

SEQ ID NO: 15 - DNA, 28 bp, Artificial Sequence
  crRNA_ALS_GT/TG protospacer oligonucleotide
agatggtatggttgtccaatgggaagat

SEQ ID NO: 16 - DNA, 28 bp, Artificial Sequence
  crRNA_ALS_G/T protospacer oligonucleotide
ggccatcttcccattggacaaccatacc

SEQ ID NO: 17 - DNA, 28 bp, Artificial Sequence
  crRNA_ALS_In1 protospacer oligonucleotide
agatggctatggatgttgtgttgcatca

SEQ ID NO: 18 - DNA, 28 bp, Artificial Sequence
  crRNA_ALS_In1 protospacer oligonucleotide
ggcctgatgcaacacaacatccatagcc

SEQ ID NO: 19 - DNA, 28 bp, Artificial Sequence
  crRNA_ALS_In2 protospacer oligonulceotide
agattacgtggttcggttgatcatatag

SEQ ID NO: 20 - DNA, 28 bp, Artificial Sequence
  crRNA_ALS_In2 protospacer oligonucleotide
ggccctatatgatcaaccgaaccacgta

SEQ ID NO: 21 - DNA, 28 bp, Artificial Sequence
  crRNA_CYP_Del protospacer oligonucleotide
agattacgtggttcggttgatcatatag

SEQ ID NO: 22 - DNA, 28 bp, Artificial Sequence
  crRNA_CYP_Del protospacer oligonucleotide
ggccctatatgatcaaccgaaccacgta

SEQ ID NO: 23 - DNA, 16862 bp, Artificial Sequence
  pZFNnptII_LbCpf1_crRNA cassette
ccggattgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgaagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccattttctgtgattttggatgtgtttttgtatggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcattccggactgtggggccatatcccagaactggttgagtcggtccaacacctgggtgccaatcatgtcgatggtggggtatggccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagaggccatggcggccgcactagtggatcccccgggatgcatgaattcaagcttatcgatgtcgacaggccttaagggccagatcccccgggctgcagaagcctatactgtacttaacttgattgcataattacttgatcatagactcatagtaaacttgattacacagataagtgaagaaacaaaccaattcaagacataaccaaagagaggtgaaagactgttttatatgtctaacattgcaccttaatatcacactgttagttcctttcttacttaaattcaacccattaaagtaaaaacaacagataataataatttgagaatgaacaaaaggaccatatcatttattaactcttatccatccatttgcattttgatgtccgaaaacaaaaactgaaagaacacagtaaattacaagcagaacaaatgatagaagaaaacagcttttccaatgccataatactcaaacttagtaggattctggtgtgtgggcaatgaaactgatgcattgaacttgacgaacgttgtcgaaaccgatgatacggacgaaagctgggaggcctggtcccattcgccatgccgaagcatgttgcccagccggcgccagcgaggaggctgggaccatgccggccaggtcttccgcggtcagactgagcgaagactcatctacacttagtagaaattagacgagcttactcgtttcgtcctcacggactcatcagtaatttggatccgctgcacatacataacatatcaagatcagaacacacatatacacacacaaatacaatcaagtcaacaactccaaaaagtccagatctacatatatacatacgtaaataacaaaatcatgtaaataatcacaatcatgtaatccagatctatgcacatatatatatacacaattaataaaaaaaatgatataacagatctatatctatgtatgtaacaacacaatcagatgagagaagtgatgttttcagatctgtatacatacaaacacaaacagatgaacaattgatacgtagatccatatgtatacgtacaattagctacacgattaaatgaaaaaaatcaacgatttcggattggtacacacaaacgcaacaatatgaagaaattcatatctgattagatataaacataaccacgtgtagatacacagtcaaatcaacaaatttatagcttctaaacggatgagatgaacaagataaagatattcacataaggcatacataagataagcagattaacaaactagcaataatacatacctaattaaaacaaggaataacagagagagagagagagagagagatttaccttgaaaatgaagaggagaagagaggatttcttaaaattgggggtagagaaagaaagatgatgaattgtgagaaaggagagatagaagggggggttgtatatataggctgtagaagattatttttgtgtttgaggcggtgaaggaagaggggatctgactatgacacgtttgcggttacgtatttcgataggagtctttcaacgcttaacgccgttactctatatgaccgtttgggccgtaacggggccgtttgttaacgctgatgttgattcttttctttctttctttcttccttttttaaagaagcaattgtacaatcgttgctagctgtcaaacggataattcggatacggatatgcctatattcatatccgtaatttttctgcaggaattcgatctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcgaattcggcgcgccatggctcctaagaagaagaggaaggttagcaagctcgagaagtttaccaactgctacagcctctctaagaccctcaggttcaaggctatccctgtgggaaagacccaagagaatatcgacaacaagaggctcctcgtcgaggatgagaagagagctgaagattacaagggcgtgaagaagctcctcgacaggtactacctcagcttcatcaacgatgtgctccacagcatcaagctcaagaacctcaacaactacatcagcctcttccgtaagaaaaccaggaccgagaaagagaacaaagagcttgagaacctcgagatcaacctccgtaaagagatcgccaaggctttcaagggaaacgagggatacaagagcctcttcaagaaggatattatcgagacaatcctgcctgagttcctggacgataaggatgagatcgctctcgtgaacagcttcaacggattcactactgccttcaccggattcttcgacaacagggaaaacatgttcagcgaagaggccaagagcacctctatcgctttcagatgcatcaacgagaacctcacgcgttacatcagcaacatggacatcttcgagaaggtggacgccatcttcgataagcacgaggtgcaagaaatcaaagagaagatcctcaacagcgactacgacgtcgaggacttttttgaaggggagttcttcaacttcgttctcacccaagagggcatcgacgtgtacaacgctattatcggaggattcgtgaccgagtctggggagaagattaagggactcaacgagtacatcaacctgtacaaccagaaaacgaagcagaagctcccgaagttcaagccgctctacaagcaggttctctctgatcgtgagagcctctcattttacggtgagggttacacctctgacgaggaagtgcttgaggttttccgtaacaccctcaacaagaacagcgagatcttctcgtccatcaagaagttggagaaacttttcaagaacttcgacgagtacagcagcgctgggatcttcgttaagaacggacctgctatcagcaccatcagcaaggatattttcggcgagtggaacgtgatcagggacaagtggaatgctgagtacgatgacatccacctcaagaagaaggctgtcgtcactgagaagtacgaggatgacaggcgtaagtcgttcaagaagatcggctctttcagcctcgagcagcttcaagaatacgctgatgctgatctcagcgtggtcgagaagctcaaagagatcatcatccagaaggtcgacgagatctacaaggtgtacgggtcctctgagaagttgttcgatgctgatttcgtcctcgagaagagtctgaagaagaacgacgctgtcgtcgcgatcatgaaggatttgctcgacagcgtgaagtccttcgagaactatatcaaggccttcttcggagagggcaaagagactaatagggacgagtctttctacggggatttcgtgctcgcttacgatatcctcctcaaggtggaccatatctacgacgccatcagaaactacgtgacccagaagccttacagcaaggacaagttcaagttgtactttcagaacccgcagttcatgggcggatgggacaaagacaaagagacagattacagggccaccatcctcaggtacgggtctaagtactacctggccatcatggacaagaaatacgccaagtgcctccaaaagatcgacaaggatgacgtgaacgggaactatgagaagatcaactacaagctccttccgggaccgaacaagatgcttcctaaggtgttcttcagcaagaaatggatggcctactacaacccgtctgaggacatccagaaaatctacaagaacgggaccttcaagaaaggcgacatgttcaacctcaacgactgccacaagctcatcgatttcttcaaggacagcatctcgcgttacccgaagtggtctaacgcttacgactttaacttcagcgagacagaaaagtacaaggatatcgccgggttctaccgtgaggttgaggaacagggttacaaggttagcttcgagagcgcctccaagaaagaggttgacaagttggtcgaagagggcaagctctacatgttccagatctataacaaggacttctccgacaagagccacggaactcctaacctccatacgatgtacttcaagctgcttttcgacgagaacaaccacgggcagatcagactttctggtggtgctgaactcttcatgcgtagggcctcactcaagaaagaagagttggttgttcacccggccaactctccaatcgctaacaagaatcctgacaacccgaaaaagaccaccacgctgtcttacgacgtctacaaggacaaaaggttcagcgaggaccagtacgagcttcatatcccgatcgctatcaacaagtgcccgaagaacatcttcaagatcaataccgaggtgagggtgctgctcaagcacgatgataacccttacgtgatcggaatcgatcgtggtgagagaaacctcctctacatcgttgtggtggacggaaagggaaacatcgtcgagcagtacagcctgaacgagattatcaacaatttcaacggcatcaggatcaagaccgactaccactcactcctcgataagaaagaaaaagagcgtttcgaggccaggcagaactggacttctatcgaaaacatcaaagagttgaaggccggctacatctctcaggtggtgcataagatctgcgagctggtggaaaagtacgatgctgtgatcgctcttgaggacctcaactctgggttcaagaacagtagagtgaaggttgagaagcaggtctaccaaaagttcgagaagatgctcatcgacaagctcaactacatggtggacaaaaagagcaacccttgcgctaccggtggtgctcttaagggataccagatcacgaacaagttcgagtccttcaagagcatgagcacccagaacggcttcatcttctatatccctgcttggctcaccagcaagatcgatccttctactggtttcgtgaacctgctcaagaccaagtacacctcgatcgccgacagcaagaagttcatctcgtctttcgacaggatcatgtacgtgccggaagaggatcttttcgagttcgctctcgactataagaacttcagcaggaccgacgccgactacattaagaagtggaagctctactcctacgggaaccgtatcaggatcttccgaaatccgaagaaaaacaacgtgttcgactgggaagaagtgtgcctcacctctgcctacaaagaactgttcaacaagtacggcatcaactaccagcagggtgatatcagggctcttttgtgcgagcagagcgacaaggcattctacagctcattcatggccctcatgtctctcatgctccagatgaggaactctatcaccggaaggaccgatgtggacttccttatctctccggtcaagaactctgacgggatcttctacgacagccgtaactatgaggctcaagagaacgctatcctgccgaagaatgctgatgcaaacggggcttacaacattgcgagaaaggttctctgggctatcgggcagtttaagaaagcggaagatgagaagctcgacaaggtgaagatcgccatctccaacaaagagtggcttgagtacgctcagacctccgttaagcacaagaggcctgctgctactaagaaagctggccaggccaaaaagaagaagtgaggcgcgccgagctccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggcttgagctccctaggatcaagcttatcgatgtcgacaggccttaagggccagatcttgggcccggtacccgatcagattgtcgtttcccgccttcggtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatatcgcggtgcatctcggtcttgcctgggcagtcgccgccgacgccgttgatgtggacgccgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcaggggtacgtcgaggtcgatccaacccctccgctgctatagtgcagtcggcttctgacgttcagtgcagccgtcgtctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcgcgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctcgcggcctggccggcgagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggcgagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccggtgggtatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatagcttgcttggtcgttccgcgtgaacgtcggctcgattgtacctgcgttcaaatactttgcgatcgtgttgcgcgcctgcccggtgcgtcggctgatctcacggatcgactgcttctctcgcaacgccatccgacggatgatgtttaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcggcggcaggatatattcaattgtaaatggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagttcgagcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgtttctatgcttcaattctgctctatagatgttcttctagtcttaacttttagaaggcacaacatttgaaatgttggtaatttttctgctgggtagtgaaccattgtaatttgtgtttgcttatattatccgttgggtttctctatgttatctgtggggccatatcccagaactggttgagtcggtccaacacctgggtgccaatcatgtcgatggtggggtatggt

SEQ ID NO: 24 - DNA, 2001 bp, Artificial Sequence
  Repair-Template ALS G/T
ggtaggcctggacctgttttgattgatcttcctaaagatattcagcagcaattggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgatcgtgtgaccgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaagtggagagaagaattaggtgagcagaagaaggaattcccactgagttttaagacatttggggatgcaattcctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaattggaagataggttctataaagctaaccgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattgatcggtttaatgacggttggaaccatttaaagagggtaagctatattactgtatgtatattagtatgttcctggataatttagaagcttttgtctgttgtcttttgcagtttatgaagttagtttgctgttgtcatgttacttgttactttaaaaagctttttgtagtttttgagcaactagtatggaatgctcttcctgtattgcttggaaaattcacaaaagtggtttttcggctatggatgttgtgttgcatcatgcatatatagcttgatatactagttggcttggtgcatctttaacatatactaatgagactacgacagcaattgccaattagttggcttgacataattcttagtctgccactagaaatcttgcttcttttttttccctcatttgttgaaagtccctgttgcgacctgacatgggagcattggagattgtttagcaatagcagttgcagttaggtgactacagtcatcttccaaatatgaatactctctggaggggaggaggttttacaaaatatgagtttttaacacatgagaaacttatattaaacaaggttgagtcaccccatatttttcaaagttgacttttgtctgatttgggtgactatgcctgttgtatgcaataaactcgatgtacataagacttgtataatccaatctaacccttcctggctgattatgaaaccgagtcggcta

SEQ ID NO: 25 - DNA, 27 bp, Artificial Sequence
  5′ALS_G/T primer
gttttggatgtagaggatattcctaga

SEQ ID NO: 26 - DNA, 23 bp, Artificial Sequence
  ALS_G/T_Rv primer
cagggaagatatcagcagatttg

SEQ ID NO: 27 - DNA, 23 bp, Artificial Sequence
  ALS_G/T_Fw primer
ctacaattagggtggaaaatctc

SEQ ID NO: 28 - DNA, 21 bp, Artificial Sequence
  3′ALS_G/T primer
ctctagtggtcacctggcatc

SEQ ID NO: 29 - DNA, 20 bp, Artificial Sequence
  pSeq_LbCpf1_F4 primer
accactcactcctcgataag

SEQ ID NO: 30 - DNA, 22 bp, Artificial Sequence
  pSeq_LbCpf1_R3 primer
tagacctgcttctcaaccttca

SEQ ID NO: 31 - DNA, 21 bp, Artificial Sequence
  pSeq_Ribozyme_F primer
tgcagcggatccaaattactg

SEQ ID NO: 32 - DNA, 18 bp, Artificial Sequence
  pSeq_Ribozyme_R primer
cctggtcccattcgccat

SEQ ID NO: 33 - DNA, 20 bp, Artificial Sequence
  pSeq_tDT_F primer
ttacaagaagctgtccttcc

SEQ ID NO: 34 - DNA, 20 bp, Artificial Sequence
  primer
gtactgttccacgatggtgt

SEQ ID NO: 35 - DNA, 2010 bp, Artificial Sequence
  Repair-Template ALS 9bp Insertion
gaatttggatttctccaagtggagagaagaattaggtgagcagaagaaggaattcccactgagttttaagacatttggggatgcaattcctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaatgggaagataggttctataaagctaaccgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattgatcggtttaatgacggttggaaccatttaaagagggtaagctatattactgtatgtatattagtatgttcctggataatttagaagcttttgtctgttgtcttttgcagtttatgaagttagtttgctgttgtcatgttacttgttactttaaaaagctttttgtagtttttgagcaactagtatggaatgctcttcctgtattgcttggaaaattcacaaaagtggtttttcggctatggatgttgtgttgcatcatgcatatatagcttgatatactagttggctgcactatcgtggtgcatctttaacatatactaatgagactacgacagcaattgccaattagttggcttgacataattcttagtctgccactagaaatcttgcttcttttttttccctcatttgttgaaagtccctgttgcgacctgacatgggagcattggagattgtttagcaatagcagttgcagttaggtgactacagtcatcttccaaatatgaatactctctggaggggaggaggttttacaaaatatgagtttttaacacatgagaaacttatattaaacaaggttgagtcaccccatatttttcaaagttgacttttgtctgatttgggtgactatgcctgttgtatgcaataaactcgatgtacataagacttgtataatccaatctaacccttcctggctgattatgaaaccgagtcggctaatttgttgcttgatcttcatgtgtgagcctgatgccaggtgaccactagaggagtacctttcattgagatatcgattcggttaattggttcctcatatgggtctcaaaactgaatttttctcaggctctcttctaaccagttgttgaattttatgaaacttcagtccagttaaaactttgatcccggatcagaatttctctgagttgttcttccgcctctcagttccactgttccagtgttcttggtcctcaacctgtactctgagctatttctgcaaacacctgaagttcctgctgcccttggacaaatacagaatgcagcattagcatttatttaagacaagatgaatacttgctcccctgttagttatgctaattctgcattagtattctttaattttaattagatactgcaatccgtgaactctgcagtttcttgatctctctctgtttcaatttctcctatcttatcgcccatttctttaggctttcgttatctatcgtctaattcaagagtaggtaactaggtacgggataaaactttttcatgaagaacctatgttcccgtattatgctgt

SEQ ID NO: 36 - DNA, 24 bp, Artificial Sequence
  5′ALS_Insertion primer
gtgctgatgttaaattggcattgc

SEQ ID NO: 37 - DNA, 23 bp, Artificial Sequence
  ALS_Insertion_Rv primer
ctagtggcagactaagaattatg

SEQ ID NO: 38 - DNA, 23 bp, Artificial Sequence
  ALS_Insertion_Fw primer
gaatgctcttcctgtattgcttg

SEQ ID NO: 39 - DNA, 24 bp, Artificial Sequence
  3′ALS_Insertion primer
cagttcaacacaaaagaagttgtc

SEQ ID NO: 40 - DNA, 2000 bp, Artificial Sequence
  Repair-Template CYP Deletionsmarker
tagagtttttcgatttacgattgtggtaaattaattgtaattgatcggttgtcttgtagtcaagaaatgacgtatgaatcaatttagggcatgttctcttcggcataaaacagctgaactgaattgaactgaactgaaatgaatagtgatatgtgagagtaaaagtattgtcaagagctgaactgagctgaactgaacggatctgaactgaactgatctaatctgaactaatctgaactgaactgaactgaattgaactgaaaataagctagggaaaacagacccttactactattatataacctcgtttaaatattaggaaattaaaaaaataattatatttctttatactttattaacctattgaggtttttatattgactcccaaatactattttatagatcatgccatgttaatgagcaaactactttctcacatctttataggagaaaaagtagatcactcactagcatatcatgaccagcgaaaccaaccaacgctaatagttttattgcttccattagagataagagttaactaataataccatctttgtgaaattttgatggattttggaactttagcaatatatttactgtgtgcactttttgctaccaaaattttataccaatggctcaagtcctacttatacacaacatacaaactccctcccggcccaccaaggtggcccttatttggaaacctccttcaactagggccacttccccaccgcgatttcgcctcattttgtgaaaaatatgggcctttagtctacataaggcttggtaatgtggatgccataaccactaatgatccagaaatcatacgtgagatcttagttcgacaagatgatgtatttgcgtctcgtccacataccttagccgcaacccacttggcttacaatagtggtgatgtggccttggctccaatgggaccaaaatggaaaagaatgagaaggatatgcatggagcacttgctcacaactagacgacttgaacttcatagggctgatgaggcacgacatttggtccaagacgtattaactcgttcccacaaagataaagttgttaatttgagggaagtgttaggtgcattttctatgaataacgtgactagaatgttgctagggaagcaatactttggggccgggacggcgggcccacaagaggctctagagtttatgcatataacacatgagttgttttggttactaggcttgatttacttgggtgattatttgcctttttggaggtgggttgatccatatggatgtgaaaagaaaatgagggaagttgaaaaaagggtagatgatttccatcgcaaaattatagaggaacataggaaggagaagaaaaggaaagaagaaatgggagtgaatgagggtgaaatggattttgtagatattttgttggctttgcctggtgaaaatggaaatgagcatatggatgatgcagatattaaagctctaattcaggtaattcatgtataatttgaatgtgatcgatacaaagtttgatagaaaacatatttgcataaatatatggttgccctactagacccaataaaatacataattattgccttactagttgaaagttgaaacaacctagctaccattttgttgtgattatcattagccaaccaaaattatttcttgcatccatatattaatgttgagatcagagtcggcatatttacaattacttgtaacattttaagcaaacaaattaaaatattttttggcaagtccattttattgaataatacctatatcttaaaatgaattcttggtcatgtacacttgcctttcaaggtaccaatatttgaccatatgtaattactattaacaaatttgataaaatctaataatatgtaaatatacattcacgcacatattagaaacaaagatcacaaatgataatgcaaaataacttatttgaactaatgttgtgaagttaaatttggaacaaaaggtatatttgtattgccgaattttaat

SEQ ID NO: 41 - DNA, 28 bp, Artificial Sequence
  5′CYP_Deletion primer
gtctttacatagcaaaacaatattgaag

SEQ ID NO: 42 - DNA, 25 bp, Artificial Sequence
  CYP_Deletion_Rv primer
ctaacacttccctcaaattaacaac

SEQ ID NO: 43 - DNA, 23 bp, Artificial Sequence
  CYP_Deletion_Fw primer
caatagtggtgatgtggccttgg

SEQ ID NO: 44 - DNA, 25 bp, Artificial Sequence
  3′CYP_Deletion primer
ggtaactagtaaaagtatactcatc

SEQ ID NO: 45 - DNA, 24 bp, Artificial Sequence
  BvCYP703A2_WT_fwd primer
tagacgacttgaactatttgtgag

SEQ ID NO: 46 - DNA, 24 bp, Artificial Sequence
  BvCYP703A2_gst_fwd primer
tagacgacttgaacttcatagggc

SEQ ID NO: 47 - DNA, 23 bp, Artificial Sequence
  BvCYP703A2_rev primer
aaagtattgcttccctagcaaca

SEQ ID NO: 48 - DNA, 21 bp, Artificial Sequence
  BvALS_WT_fwd primer
actagttggcttggtgcatct

SEQ ID NO: 49 - DNA, 23 bp, Artificial Sequence
  BvALS_SU_res_fwd primer
actagttggctgcactatcgtgc

SEQ ID NO: 50 - DNA, 20 bp, Artificial Sequence
  BvALS_rev primer
ccaatgctcccatgtcaggt

SEQ ID NO: 51 - DNA, 3759 bp, Lachnospiraceae bacterium
atggctcctaagaagaagaggaaggttagcaagctcgagaagtttaccaactgctacagcctctctaagaccctcaggttcaaggctatccctgtgggaaagacccaagagaatatcgacaacaagaggctcctcgtcgaggatgagaagagagctgaagattacaagggcgtgaagaagctcctcgacaggtactacctcagcttcatcaacgatgtgctccacagcatcaagctcaagaacctcaacaactacatcagcctcttccgtaagaaaaccaggaccgagaaagagaacaaagagcttgagaacctcgagatcaacctccgtaaagagatcgccaaggctttcaagggaaacgagggatacaagagcctcttcaagaaggatattatcgagacaatcctgcctgagttcctggacgataaggatgagatcgctctcgtgaacagcttcaacggattcactactgccttcaccggattcttcgacaacagggaaaacatgttcagcgaagaggccaagagcacctctatcgctttcagatgcatcaacgagaacctcacgcgttacatcagcaacatggacatcttcgagaaggtggacgccatcttcgataagcacgaggtgcaagaaatcaaagagaagatcctcaacagcgactacgacgtcgaggacttttttgaaggggagttcttcaacttcgttctcacccaagagggcatcgacgtgtacaacgctattatcggaggattcgtgaccgagtctggggagaagattaagggactcaacgagtacatcaacctgtacaaccagaaaacgaagcagaagctcccgaagttcaagccgctctacaagcaggttctctctgatcgtgagagcctctcattttacggtgagggttacacctctgacgaggaagtgcttgaggttttccgtaacaccctcaacaagaacagcgagatcttctcgtccatcaagaagttggagaaacttttcaagaacttcgacgagtacagcagcgctgggatcttcgttaagaacggacctgctatcagcaccatcagcaaggatattttcggcgagtggaacgtgatcagggacaagtggaatgctgagtacgatgacatccacctcaagaagaaggctgtcgtcactgagaagtacgaggatgacaggcgtaagtcgttcaagaagatcggctctttcagcctcgagcagcttcaagaatacgctgatgctgatctcagcgtggtcgagaagctcaaagagatcatcatccagaaggtcgacgagatctacaaggtgtacgggtcctctgagaagttgttcgatgctgatttcgtcctcgagaagagtctgaagaagaacgacgctgtcgtcgcgatcatgaaggatttgctcgacagcgtgaagtccttcgagaactatatcaaggccttcttcggagagggcaaagagactaatagggacgagtctttctacggggatttcgtgctcgcttacgatatcctcctcaaggtggaccatatctacgacgccatcagaaactacgtgacccagaagccttacagcaaggacaagttcaagttgtactttcagaacccgcagttcatgggcggatgggacaaagacaaagagacagattacagggccaccatcctcaggtacgggtctaagtactacctggccatcatggacaagaaatacgccaagtgcctccaaaagatcgacaaggatgacgtgaacgggaactatgagaagatcaactacaagctccttccgggaccgaacaagatgcttcctaaggtgttcttcagcaagaaatggatggcctactacaacccgtctgaggacatccagaaaatctacaagaacgggaccttcaagaaaggcgacatgttcaacctcaacgactgccacaagctcatcgatttcttcaaggacagcatctcgcgttacccgaagtggtctaacgcttacgactttaacttcagcgagacagaaaagtacaaggatatcgccgggttctaccgtgaggttgaggaacagggttacaaggttagcttcgagagcgcctccaagaaagaggttgacaagttggtcgaagagggcaagctctacatgttccagatctataacaaggacttctccgacaagagccacggaactcctaacctccatacgatgtacttcaagctgcttttcgacgagaacaaccacgggcagatcagactttctggtggtgctgaactcttcatgcgtagggcctcactcaagaaagaagagttggttgttcacccggccaactctccaatcgctaacaagaatcctgacaacccgaaaaagaccaccacgctgtcttacgacgtctacaaggacaaaaggttcagcgaggaccagtacgagcttcatatcccgatcgctatcaacaagtgcccgaagaacatcttcaagatcaataccgaggtgagggtgctgctcaagcacgatgataacccttacgtgatcggaatcgatcgtggtgagagaaacctcctctacatcgttgtggtggacggaaagggaaacatcgtcgagcagtacagcctgaacgagattatcaacaatttcaacggcatcaggatcaagaccgactaccactcactcctcgataagaaagaaaaagagcgtttcgaggccaggcagaactggacttctatcgaaaacatcaaagagttgaaggccggctacatctctcaggtggtgcataagatctgcgagctggtggaaaagtacgatgctgtgatcgctcttgaggacctcaactctgggttcaagaacagtagagtgaaggttgagaagcaggtctaccaaaagttcgagaagatgctcatcgacaagctcaactacatggtggacaaaaagagcaacccttgcgctaccggtggtgctcttaagggataccagatcacgaacaagttcgagtccttcaagagcatgagcacccagaacggcttcatcttctatatccctgcttggctcaccagcaagatcgatccttctactggtttcgtgaacctgctcaagaccaagtacacctcgatcgccgacagcaagaagttcatctcgtctttcgacaggatcatgtacgtgccggaagaggatcttttcgagttcgctctcgactataagaacttcagcaggaccgacgccgactacattaagaagtggaagctctactcctacgggaaccgtatcaggatcttccgaaatccgaagaaaaacaacgtgttcgactgggaagaagtgtgcctcacctctgcctacaaagaactgttcaacaagtacggcatcaactaccagcagggtgatatcagggctcttttgtgcgagcagagcgacaaggcattctacagctcattcatggccctcatgtctctcatgctccagatgaggaactctatcaccggaaggaccgatgtggacttccttatctctccggtcaagaactctgacgggatcttctacgacagccgtaactatgaggctcaagagaacgctatcctgccgaagaatgctgatgcaaacggggcttacaacattgcgagaaaggttctctgggctatcgggcagtttaagaaagcggaagatgagaagctcgacaaggtgaagatcgccatctccaacaaagagtggcttgagtacgctcagacctccgttaagcacaagaggcctgctgctactaagaaagctggccaggccaaaaagaagaagtga

SEQ ID NO: 52 - DNA, 5228 bp, Artificial Sequence
  PcUbiCpf1TPea expression cassette
aaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcgaattcggcgcgccatggctcctaagaagaagaggaaggttagcaagctcgagaagtttaccaactgctacagcctctctaagaccctcaggttcaaggctatccctgtgggaaagacccaagagaatatcgacaacaagaggctcctcgtcgaggatgagaagagagctgaagattacaagggcgtgaagaagctcctcgacaggtactacctcagcttcatcaacgatgtgctccacagcatcaagctcaagaacctcaacaactacatcagcctcttccgtaagaaaaccaggaccgagaaagagaacaaagagcttgagaacctcgagatcaacctccgtaaagagatcgccaaggctttcaagggaaacgagggatacaagagcctcttcaagaaggatattatcgagacaatcctgcctgagttcctggacgataaggatgagatcgctctcgtgaacagcttcaacggattcactactgccttcaccggattcttcgacaacagggaaaacatgttcagcgaagaggccaagagcacctctatcgctttcagatgcatcaacgagaacctcacgcgttacatcagcaacatggacatcttcgagaaggtggacgccatcttcgataagcacgaggtgcaagaaatcaaagagaagatcctcaacagcgactacgacgtcgaggacttttttgaaggggagttcttcaacttcgttctcacccaagagggcatcgacgtgtacaacgctattatcggaggattcgtgaccgagtctggggagaagattaagggactcaacgagtacatcaacctgtacaaccagaaaacgaagcagaagctcccgaagttcaagccgctctacaagcaggttctctctgatcgtgagagcctctcattttacggtgagggttacacctctgacgaggaagtgcttgaggttttccgtaacaccctcaacaagaacagcgagatcttctcgtccatcaagaagttggagaaacttttcaagaacttcgacgagtacagcagcgctgggatcttcgttaagaacggacctgctatcagcaccatcagcaaggatattttcggcgagtggaacgtgatcagggacaagtggaatgctgagtacgatgacatccacctcaagaagaaggctgtcgtcactgagaagtacgaggatgacaggcgtaagtcgttcaagaagatcggctctttcagcctcgagcagcttcaagaatacgctgatgctgatctcagcgtggtcgagaagctcaaagagatcatcatccagaaggtcgacgagatctacaaggtgtacgggtcctctgagaagttgttcgatgctgatttcgtcctcgagaagagtctgaagaagaacgacgctgtcgtcgcgatcatgaaggatttgctcgacagcgtgaagtccttcgagaactatatcaaggccttcttcggagagggcaaagagactaatagggacgagtctttctacggggatttcgtgctcgcttacgatatcctcctcaaggtggaccatatctacgacgccatcagaaactacgtgacccagaagccttacagcaaggacaagttcaagttgtactttcagaacccgcagttcatgggcggatgggacaaagacaaagagacagattacagggccaccatcctcaggtacgggtctaagtactacctggccatcatggacaagaaatacgccaagtgcctccaaaagatcgacaaggatgacgtgaacgggaactatgagaagatcaactacaagctccttccgggaccgaacaagatgcttcctaaggtgttcttcagcaagaaatggatggcctactacaacccgtctgaggacatccagaaaatctacaagaacgggaccttcaagaaaggcgacatgttcaacctcaacgactgccacaagctcatcgatttcttcaaggacagcatctcgcgttacccgaagtggtctaacgcttacgactttaacttcagcgagacagaaaagtacaaggatatcgccgggttctaccgtgaggttgaggaacagggttacaaggttagcttcgagagcgcctccaagaaagaggttgacaagttggtcgaagagggcaagctctacatgttccagatctataacaaggacttctccgacaagagccacggaactcctaacctccatacgatgtacttcaagctgcttttcgacgagaacaaccacgggcagatcagactttctggtggtgctgaactcttcatgcgtagggcctcactcaagaaagaagagttggttgttcacccggccaactctccaatcgctaacaagaatcctgacaacccgaaaaagaccaccacgctgtcttacgacgtctacaaggacaaaaggttcagcgaggaccagtacgagcttcatatcccgatcgctatcaacaagtgcccgaagaacatcttcaagatcaataccgaggtgagggtgctgctcaagcacgatgataacccttacgtgatcggaatcgatcgtggtgagagaaacctcctctacatcgttgtggtggacggaaagggaaacatcgtcgagcagtacagcctgaacgagattatcaacaatttcaacggcatcaggatcaagaccgactaccactcactcctcgataagaaagaaaaagagcgtttcgaggccaggcagaactggacttctatcgaaaacatcaaagagttgaaggccggctacatctctcaggtggtgcataagatctgcgagctggtggaaaagtacgatgctgtgatcgctcttgaggacctcaactctgggttcaagaacagtagagtgaaggttgagaagcaggtctaccaaaagttcgagaagatgctcatcgacaagctcaactacatggtggacaaaaagagcaacccttgcgctaccggtggtgctcttaagggataccagatcacgaacaagttcgagtccttcaagagcatgagcacccagaacggcttcatcttctatatccctgcttggctcaccagcaagatcgatccttctactggtttcgtgaacctgctcaagaccaagtacacctcgatcgccgacagcaagaagttcatctcgtctttcgacaggatcatgtacgtgccggaagaggatcttttcgagttcgctctcgactataagaacttcagcaggaccgacgccgactacattaagaagtggaagctctactcctacgggaaccgtatcaggatcttccgaaatccgaagaaaaacaacgtgttcgactgggaagaagtgtgcctcacctctgcctacaaagaactgttcaacaagtacggcatcaactaccagcagggtgatatcagggctcttttgtgcgagcagagcgacaaggcattctacagctcattcatggccctcatgtctctcatgctccagatgaggaactctatcaccggaaggaccgatgtggacttccttatctctccggtcaagaactctgacgggatcttctacgacagccgtaactatgaggctcaagagaacgctatcctgccgaagaatgctgatgcaaacggggcttacaacattgcgagaaaggttctctgggctatcgggcagtttaagaaagcggaagatgagaagctcgacaaggtgaagatcgccatctccaacaaagagtggcttgagtacgctcagacctccgttaagcacaagaggcctgctgctactaagaaagctggccaggccaaaaagaagaagtgaggcgcgccgagctccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggctt

SEQ ID NO: 53 - DNA, 37 bp, Artificial Sequence
  HH ribozyme encoding sequence
ctgatgagtccgtgaggacgaaacgagtaagctcgtc

SEQ ID NO: 54 - DNA, 68 bp, Hepatitis Delta Virus
ggccggcatggtcccagcctcctcgctggcgccggctgggcaacatgcttcggcatggcgaatgggac

SEQ ID NO: 55 - DNA, 15245 bp, Artificial Sequence
  pZFNnptII_Cpf1 target vector
tgcaggaattcgatctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcgaattcggcgcgccatggctcctaagaagaagaggaaggttagcaagctcgagaagtttaccaactgctacagcctctctaagaccctcaggttcaaggctatccctgtgggaaagacccaagagaatatcgacaacaagaggctcctcgtcgaggatgagaagagagctgaagattacaagggcgtgaagaagctcctcgacaggtactacctcagcttcatcaacgatgtgctccacagcatcaagctcaagaacctcaacaactacatcagcctcttccgtaagaaaaccaggaccgagaaagagaacaaagagcttgagaacctcgagatcaacctccgtaaagagatcgccaaggctttcaagggaaacgagggatacaagagcctcttcaagaaggatattatcgagacaatcctgcctgagttcctggacgataaggatgagatcgctctcgtgaacagcttcaacggattcactactgccttcaccggattcttcgacaacagggaaaacatgttcagcgaagaggccaagagcacctctatcgctttcagatgcatcaacgagaacctcacgcgttacatcagcaacatggacatcttcgagaaggtggacgccatcttcgataagcacgaggtgcaagaaatcaaagagaagatcctcaacagcgactacgacgtcgaggacttttttgaaggggagttcttcaacttcgttctcacccaagagggcatcgacgtgtacaacgctattatcggaggattcgtgaccgagtctggggagaagattaagggactcaacgagtacatcaacctgtacaaccagaaaacgaagcagaagctcccgaagttcaagccgctctacaagcaggttctctctgatcgtgagagcctctcattttacggtgagggttacacctctgacgaggaagtgcttgaggttttccgtaacaccctcaacaagaacagcgagatcttctcgtccatcaagaagttggagaaacttttcaagaacttcgacgagtacagcagcgctgggatcttcgttaagaacggacctgctatcagcaccatcagcaaggatattttcggcgagtggaacgtgatcagggacaagtggaatgctgagtacgatgacatccacctcaagaagaaggctgtcgtcactgagaagtacgaggatgacaggcgtaagtcgttcaagaagatcggctctttcagcctcgagcagcttcaagaatacgctgatgctgatctcagcgtggtcgagaagctcaaagagatcatcatccagaaggtcgacgagatctacaaggtgtacgggtcctctgagaagttgttcgatgctgatttcgtcctcgagaagagtctgaagaagaacgacgctgtcgtcgcgatcatgaaggatttgctcgacagcgtgaagtccttcgagaactatatcaaggccttcttcggagagggcaaagagactaatagggacgagtctttctacggggatttcgtgctcgcttacgatatcctcctcaaggtggaccatatctacgacgccatcagaaactacgtgacccagaagccttacagcaaggacaagttcaagttgtactttcagaacccgcagttcatgggcggatgggacaaagacaaagagacagattacagggccaccatcctcaggtacgggtctaagtactacctggccatcatggacaagaaatacgccaagtgcctccaaaagatcgacaaggatgacgtgaacgggaactatgagaagatcaactacaagctccttccgggaccgaacaagatgcttcctaaggtgttcttcagcaagaaatggatggcctactacaacccgtctgaggacatccagaaaatctacaagaacgggaccttcaagaaaggcgacatgttcaacctcaacgactgccacaagctcatcgatttcttcaaggacagcatctcgcgttacccgaagtggtctaacgcttacgactttaacttcagcgagacagaaaagtacaaggatatcgccgggttctaccgtgaggttgaggaacagggttacaaggttagcttcgagagcgcctccaagaaagaggttgacaagttggtcgaagagggcaagctctacatgttccagatctataacaaggacttctccgacaagagccacggaactcctaacctccatacgatgtacttcaagctgcttttcgacgagaacaaccacgggcagatcagactttctggtggtgctgaactcttcatgcgtagggcctcactcaagaaagaagagttggttgttcacccggccaactctccaatcgctaacaagaatcctgacaacccgaaaaagaccaccacgctgtcttacgacgtctacaaggacaaaaggttcagcgaggaccagtacgagcttcatatcccgatcgctatcaacaagtgcccgaagaacatcttcaagatcaataccgaggtgagggtgctgctcaagcacgatgataacccttacgtgatcggaatcgatcgtggtgagagaaacctcctctacatcgttgtggtggacggaaagggaaacatcgtcgagcagtacagcctgaacgagattatcaacaatttcaacggcatcaggatcaagaccgactaccactcactcctcgataagaaagaaaaagagcgtttcgaggccaggcagaactggacttctatcgaaaacatcaaagagttgaaggccggctacatctctcaggtggtgcataagatctgcgagctggtggaaaagtacgatgctgtgatcgctcttgaggacctcaactctgggttcaagaacagtagagtgaaggttgagaagcaggtctaccaaaagttcgagaagatgctcatcgacaagctcaactacatggtggacaaaaagagcaacccttgcgctaccggtggtgctcttaagggataccagatcacgaacaagttcgagtccttcaagagcatgagcacccagaacggcttcatcttctatatccctgcttggctcaccagcaagatcgatccttctactggtttcgtgaacctgctcaagaccaagtacacctcgatcgccgacagcaagaagttcatctcgtctttcgacaggatcatgtacgtgccggaagaggatcttttcgagttcgctctcgactataagaacttcagcaggaccgacgccgactacattaagaagtggaagctctactcctacgggaaccgtatcaggatcttccgaaatccgaagaaaaacaacgtgttcgactgggaagaagtgtgcctcacctctgcctacaaagaactgttcaacaagtacggcatcaactaccagcagggtgatatcagggctcttttgtgcgagcagagcgacaaggcattctacagctcattcatggccctcatgtctctcatgctccagatgaggaactctatcaccggaaggaccgatgtggacttccttatctctccggtcaagaactctgacgggatcttctacgacagccgtaactatgaggctcaagagaacgctatcctgccgaagaatgctgatgcaaacggggcttacaacattgcgagaaaggttctctgggctatcgggcagtttaagaaagcggaagatgagaagctcgacaaggtgaagatcgccatctccaacaaagagtggcttgagtacgctcagacctccgttaagcacaagaggcctgctgctactaagaaagctggccaggccaaaaagaagaagtgaggcgcgccgagctccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggcttgagctccctaggatcaagcttatcgatgtcgacaggccttaagggccagatcttgggcccggtacccgatcagattgtcgtttcccgccttcggtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatatcgcggtgcatctcggtcttgcctgggcagtcgccgccgacgccgttgatgtggacgccgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcaggggtacgtcgaggtcgatccaacccctccgctgctatagtgcagtcggcttctgacgttcagtgcagccgtcgtctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcgcgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctcgcggcctggccggcgagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggcgagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccggtgggtatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatagcttgcttggtcgttccgcgtgaacgtcggctcgattgtacctgcgttcaaatactttgcgatcgtgttgcgcgcctgcccggtgcgtcggctgatctcacggatcgactgcttctctcgcaacgccatccgacggatgatgtttaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcggcggcaggatatattcaattgtaaatggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagttcgagcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgtttctatgcttcaattctgctctatagatgttcttctagtcttaacttttagaaggcacaacatttgaaatgttggtaatttttctgctgggtagtgaaccattgtaatttgtgtttgcttatattatccgttgggtttctctatgttatctgtggggccatatcccagaactggttgagtcggtccaacacctgggtgccaatcatgtcgatggtggggtatggtccggattgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgaagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccattttctgtgattttggatgtgtttttgtatggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcattccggactgtggggccatatcccagaactggttgagtcggtccaacacctgggtgccaatcatgtcgatggtggggtatggccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagaggccatggcggccgcactagtggatcccccgggatgcatgaattcaagcttatcgatgtcgacaggccttaagggccagatcccccgggc

SEQ ID NO: 56 - DNA, 1611 bp, Artificial Sequence
  crRNA expression cassette
aaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcggatccaaattactgatgagtccgtgaggacgaaacgagtaagctcgtctaatttctactaagtgtagatgagtcttcgctcagtctgaccgcggaagacctggccggcatggtcccagcctcctcgctggcgccggctgggcaacatgcttcggcatggcgaatgggaccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggctt

SEQ ID NO: 57 - DNA, 9763 bp, Artificial Sequence
  pZFNtDTnptII plasmid
agcttatcgatgtcgacaggccttaagggccagatcttgggcccggtacccgatcagattgtcgtttcccgccttcggtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatatcgcggtgcatctcggtcttgcctgggcagtcgccgccgacgccgttgatgtggacgccgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcaggggtacgtcgaggtcgatccaacccctccgctgctatagtgcagtcggcttctgacgttcagtgcagccgtcgtctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcgcgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctcgcggcctggccggcgagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggcgagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccggtgggtatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatagcttgcttggtcgttccgcgtgaacgtcggctcgattgtacctgcgttcaaatactttgcgatcgtgttgcgcgcctgcccggtgcgtcggctgatctcacggatcgactgcttctctcgcaacgccatccgacggatgatgtttaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcggcggcaggatatattcaattgtaaatggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagttcgagcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgtttctatgcttcaattctgctctatagatgttcttctagtcttaacttttagaaggcacaacatttgaaatgttggtaatttttctgctgggtagtgaaccattgtaatttgtgtttgcttatattatccgttgggtttctctatgttatctgtggggccatatcccagaactggttgagtcggtccaacacctgggtgccaatcatgtcgatggtggggtatggtccggattgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgaagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccattttctgtgattttggatgtgtttttgtatggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcattccggactgtggggccatatcccagaactggttgagtcggtccaacacctgggtgccaatcatgtcgatggtggggtatggccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagaggccatggcggccgcactagtggatcccccgggatgcatgaattca

SEQ ID NO: 58 - DNA, 45 bp, Artificial Sequence
  crRNA_ALS G/T
attaaagatgattcacatctaggtatggttgtccaatgggaagat

SEQ ID NO: 59 - DNA, 45 bp, Artificial Sequence
  crRNA_Cyp Del
attaaagatgattcacatctatacgtggttcggttgatcatatag

SEQ ID NO: 60 - DNA, 45 bp, Artificial Sequence
  crRNA_ALS In1
attaaagatgattcacatctaggctatggatgttgtgttgcatca

SEQ ID NO: 61 - DNA, 45 bp, Artificial Sequence; identical to SEQ ID NO: 59
  crRNA_ALS In2

SEQ ID NO: 62 - DNA, 1603 bp, Artificial Sequence
  crRNA_ALS_G/T_expression cassette
aaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcggatccaaattactgatgagtccgtgaggacgaaacgagtaagctcgtctaatttctactaagtgtagatccataccaacaggttacccttctaggccggcatggtcccagcctcctcgctggcgccggctgggcaacatgcttcggcatggcgaatgggaccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggctt

SEQ ID NO: 63 - DNA, 1603 bp, Artificial Sequence
  crRNA_ALS_In1 expression cassette
aaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcggatccaaattactgatgagtccgtgaggacgaaacgagtaagctcgtctaatttctactaagtgtagatccgatacctacaacacaacgtagtggccggcatggtcccagcctcctcgctggcgccggctgggcaacatgcttcggcatggcgaatgggaccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggctt

SEQ ID NO: 64 - DNA, 1603 bp, Artificial Sequence
  crRNA_ALS_In2 expression cassette
aaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcggatccaaattactgatgagtccgtgaggacgaaacgagtaagctcgtctaatttctactaagtgtagatatgcaccaagccaactagtatatcggccggcatggtcccagcctcctcgctggcgccggctgggcaacatgcttcggcatggcgaatgggaccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggctt

SEQ ID NO: 65 - DNA, 1603 bp, Artificial Sequence; identical to SEQ ID NO: 64
  crRNA_Cyp_Del expression cassette

SEQ ID NO: 66 - DNA, 5816 bp, Artificial Sequence
  pUbitDTnptII vector
agctttccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctgaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcactcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaagaaataggggttctgcgcacatttccccgaaaagtgccacctgaaattgtaaacgttaatgttttgttaaatttcgcgttaaatatttgttaaatcagcttattttttaaccagtaagcagaaaatgacaaaaatccttataaatcaaaagaatagaccgagttagttgtgagtgttgttccagtttggaacaagagtccactattaaagaacgtggactccaacgtaaaaccgtctatcagggcgatggcccactacgtgaaccatcacccaaatcaagtttttggaggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctactgggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattatcgattaaattaaccctcactaaagggagagacgaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagctgaaccctcgagatcctctcaacacaacatatacaaaacaaacgaatctcaagcaatcaagcattctacttctattgcagcaatttaaatcatttcttttaaagcaaaagcaattttctgaaaattttcaccatttacgaacgatagggcgcgatcccgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtctagaggtaccggttgttggcccatgggagcttggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggacccaagctaggccatcgatacgcgtacgtcgcgaccgcggacatgtacagagctcgagaagtactagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgca

SEQ ID NO: 67 - DNA, 12928 bp, Artificial Sequence
  pUbitDTnptII_LbCPF1_CrRNA_ALS_G/T vector
gatcctctcaacacaacatatacaaaacaaacgaatctcaagcaatcaagcattctacttctattgcagcaatttaaatcatttcttttaaagcaaaagcaattttctgaaaattttcaccatttacgaacgatagggcgcgatcccgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtctagaggtaccggttgttggcccatgggagcttggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggacccaagctaggccatcgatacgcgtacgtcgcgaccgcggacatgtacagagctcgagaagtactagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcaagcttatcgatgtcgacaggccttaagggccagatcccccgggctgcagaagcctatactgtacttaacttgattgcataattacttgatcatagactcatagtaaacttgattacacagataagtgaagaaacaaaccaattcaagacataaccaaagagaggtgaaagactgttttatatgtctaacattgcaccttaatatcacactgttagttcctttcttacttaaattcaacccattaaagtaaaaacaacagataataataatttgagaatgaacaaaaggaccatatcatttattaactcttatccatccatttgcattttgatgtccgaaaacaaaaactgaaagaacacagtaaattacaagcagaacaaatgatagaagaaaacagcttttccaatgccataatactcaaacttagtaggattctggtgtgtgggcaatgaaactgatgcattgaacttgacgaacgttgtcgaaaccgatgatacggacgaaagctgggaggcctggtcccattcgccatgccgaagcatgttgcccagccggcgccagcgaggaggctgggaccatgccggcctagaagggtaacctgttggtatggatctacacttagtagaaattaatctacacttagtagaaattagacgagcttactcgtttcgtcctcacggactcatcagtaatttggatccgctgcacatacataacatatcaagatcagaacacacatatacacacacaaatacaatcaagtcaacaactccaaaaagtccagatctacatatatacatacgtaaataacaaaatcatgtaaataatcacaatcatgtaatccagatctatgcacatatatatatacacaattaataaaaaaaatgatataacagatctatatctatgtatgtaacaacacaatcagatgagagaagtgatgttttcagatctgtatacatacaaacacaaacagatgaacaattgatacgtagatccatatgtatacgtacaattagctacacgattaaatgaaaaaaatcaacgatttcggattggtacacacaaacgcaacaatatgaagaaattcatatctgattagatataaacataaccacgtgtagatacacagtcaaatcaacaaatttatagcttctaaacggatgagatgaacaagataaagatattcacataaggcatacataagataagcagattaacaaactagcaataatacatacctaattaaaacaaggaataacagagagagagagagagagagagatttaccttgaaaatgaagaggagaagagaggatttcttaaaattgggggtagagaaagaaagatgatgaattgtgagaaaggagagatagaagggggggttgtatatataggctgtagaagattatttttgtgtttgaggcggtgaaggaagaggggatctgactatgacacgtttgcggttacgtatttcgataggagtctttcaacgcttaacgccgttactctatatgaccgtttgggccgtaacggggccgtttgttaacgctgatgttgattcttttctttctttctttcttccttttttaaagaagcaattgtacaatcgttgctagctgtcaaacggataattcggatacggatatgcctatattcatatccgtaatttttctgcaggaattcgatctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcgaattcggcgcgccatggctcctaagaagaagaggaaggttagcaagctcgagaagtttaccaactgctacagcctctctaagaccctcaggttcaaggctatccctgtgggaaagacccaagagaatatcgacaacaagaggctcctcgtcgaggatgagaagagagctgaagattacaagggcgtgaagaagctcctcgacaggtactacctcagcttcatcaacgatgtgctccacagcatcaagctcaagaacctcaacaactacatcagcctcttccgtaagaaaaccaggaccgagaaagagaacaaagagcttgagaacctcgagatcaacctccgtaaagagatcgccaaggctttcaagggaaacgagggatacaagagcctcttcaagaaggatattatcgagacaatcctgcctgagttcctggacgataaggatgagatcgctctcgtgaacagcttcaacggattcactactgccttcaccggattcttcgacaacagggaaaacatgttcagcgaagaggccaagagcacctctatcgctttcagatgcatcaacgagaacctcacgcgttacatcagcaacatggacatcttcgagaaggtggacgccatcttcgataagcacgaggtgcaagaaatcaaagagaagatcctcaacagcgactacgacgtcgaggacttttttgaaggggagttcttcaacttcgttctcacccaagagggcatcgacgtgtacaacgctattatcggaggattcgtgaccgagtctggggagaagattaagggactcaacgagtacatcaacctgtacaaccagaaaacgaagcagaagctcccgaagttcaagccgctctacaagcaggttctctctgatcgtgagagcctctcattttacggtgagggttacacctctgacgaggaagtgcttgaggttttccgtaacaccctcaacaagaacagcgagatcttctcgtccatcaagaagttggagaaacttttcaagaacttcgacgagtacagcagcgctgggatcttcgttaagaacggacctgctatcagcaccatcagcaaggatattttcggcgagtggaacgtgatcagggacaagtggaatgctgagtacgatgacatccacctcaagaagaaggctgtcgtcactgagaagtacgaggatgacaggcgtaagtcgttcaagaagatcggctctttcagcctcgagcagcttcaagaatacgctgatgctgatctcagcgtggtcgagaagctcaaagagatcatcatccagaaggtcgacgagatctacaaggtgtacgggtcctctgagaagttgttcgatgctgatttcgtcctcgagaagagtctgaagaagaacgacgctgtcgtcgcgatcatgaaggatttgctcgacagcgtgaagtccttcgagaactatatcaaggccttcttcggagagggcaaagagactaatagggacgagtctttctacggggatttcgtgctcgcttacgatatcctcctcaaggtggaccatatctacgacgccatcagaaactacgtgacccagaagccttacagcaaggacaagttcaagttgtactttcagaacccgcagttcatgggcggatgggacaaagacaaagagacagattacagggccaccatcctcaggtacgggtctaagtactacctggccatcatggacaagaaatacgccaagtgcctccaaaagatcgacaaggatgacgtgaacgggaactatgagaagatcaactacaagctccttccgggaccgaacaagatgcttcctaaggtgttcttcagcaagaaatggatggcctactacaacccgtctgaggacatccagaaaatctacaagaacgggaccttcaagaaaggcgacatgttcaacctcaacgactgccacaagctcatcgatttcttcaaggacagcatctcgcgttacccgaagtggtctaacgcttacgactttaacttcagcgagacagaaaagtacaaggatatcgccgggttctaccgtgaggttgaggaacagggttacaaggttagcttcgagagcgcctccaagaaagaggttgacaagttggtcgaagagggcaagctctacatgttccagatctataacaaggacttctccgacaagagccacggaactcctaacctccatacgatgtacttcaagctgcttttcgacgagaacaaccacgggcagatcagactttctggtggtgctgaactcttcatgcgtagggcctcactcaagaaagaagagttggttgttcacccggccaactctccaatcgctaacaagaatcctgacaacccgaaaaagaccaccacgctgtcttacgacgtctacaaggacaaaaggttcagcgaggaccagtacgagcttcatatcccgatcgctatcaacaagtgcccgaagaacatcttcaagatcaataccgaggtgagggtgctgctcaagcacgatgataacccttacgtgatcggaatcgatcgtggtgagagaaacctcctctacatcgttgtggtggacggaaagggaaacatcgtcgagcagtacagcctgaacgagattatcaacaatttcaacggcatcaggatcaagaccgactaccactcactcctcgataagaaagaaaaagagcgtttcgaggccaggcagaactggacttctatcgaaaacatcaaagagttgaaggccggctacatctctcaggtggtgcataagatctgcgagctggtggaaaagtacgatgctgtgatcgctcttgaggacctcaactctgggttcaagaacagtagagtgaaggttgagaagcaggtctaccaaaagttcgagaagatgctcatcgacaagctcaactacatggtggacaaaaagagcaacccttgcgctaccggtggtgctcttaagggataccagatcacgaacaagttcgagtccttcaagagcatgagcacccagaacggcttcatcttctatatccctgcttggctcaccagcaagatcgatccttctactggtttcgtgaacctgctcaagaccaagtacacctcgatcgccgacagcaagaagttcatctcgtctttcgacaggatcatgtacgtgccggaagaggatcttttcgagttcgctctcgactataagaacttcagcaggaccgacgccgactacattaagaagtggaagctctactcctacgggaaccgtatcaggatcttccgaaatccgaagaaaaacaacgtgttcgactgggaagaagtgtgcctcacctctgcctacaaagaactgttcaacaagtacggcatcaactaccagcagggtgatatcagggctcttttgtgcgagcagagcgacaaggcattctacagctcattcatggccctcatgtctctcatgctccagatgaggaactctatcaccggaaggaccgatgtggacttccttatctctccggtcaagaactctgacgggatcttctacgacagccgtaactatgaggctcaagagaacgctatcctgccgaagaatgctgatgcaaacggggcttacaacattgcgagaaaggttctctgggctatcgggcagtttaagaaagcggaagatgagaagctcgacaaggtgaagatcgccatctccaacaaagagtggcttgagtacgctcagacctccgttaagcacaagaggcctgctgctactaagaaagctggccaggccaaaaagaagaagtgaggcgcgccgagctccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggcttgagctccctaggatcaagctttccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctgaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcactcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaagaaataggggttctgcgcacatttccccgaaaagtgccacctgaaattgtaaacgttaatgttttgttaaatttcgcgttaaatatttgttaaatcagcttattttttaaccagtaagcagaaaatgacaaaaatccttataaatcaaaagaatagaccgagttagttgtgagtgttgttccagtttggaacaagagtccactattaaagaacgtggactccaacgtaaaaccgtctatcagggcgatggcccactacgtgaaccatcacccaaatcaagtttttggaggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctactgggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattatcgattaaattaaccctcactaaagggagagacgaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagctgaaccctcga

SEQ ID NO: 68 - DNA, 12928 bp, Artificial Sequence
  pUbitDTnptII_LbCpf1_ crRNA _ ALS_In1 vector
gatcctctcaacacaacatatacaaaacaaacgaatctcaagcaatcaagcattctacttctattgcagcaatttaaatcatttcttttaaagcaaaagcaattttctgaaaattttcaccatttacgaacgatagggcgcgatcccgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtctagaggtaccggttgttggcccatgggagcttggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggacccaagctaggccatcgatacgcgtacgtcgcgaccgcggacatgtacagagctcgagaagtactagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcaagcttatcgatgtcgacaggccttaagggccagatcccccgggctgcagaagcctatactgtacttaacttgattgcataattacttgatcatagactcatagtaaacttgattacacagataagtgaagaaacaaaccaattcaagacataaccaaagagaggtgaaagactgttttatatgtctaacattgcaccttaatatcacactgttagttcctttcttacttaaattcaacccattaaagtaaaaacaacagataataataatttgagaatgaacaaaaggaccatatcatttattaactcttatccatccatttgcattttgatgtccgaaaacaaaaactgaaagaacacagtaaattacaagcagaacaaatgatagaagaaaacagcttttccaatgccataatactcaaacttagtaggattctggtgtgtgggcaatgaaactgatgcattgaacttgacgaacgttgtcgaaaccgatgatacggacgaaagctgggaggcctggtcccattcgccatgccgaagcatgttgcccagccggcgccagcgaggaggctgggaccatgccggccactacgttgtgttgtaggtatcggatctacacttagtagaaattaatctacacttagtagaaattagacgagcttactcgtttcgtcctcacggactcatcagtaatttggatccgctgcacatacataacatatcaagatcagaacacacatatacacacacaaatacaatcaagtcaacaactccaaaaagtccagatctacatatatacatacgtaaataacaaaatcatgtaaataatcacaatcatgtaatccagatctatgcacatatatatatacacaattaataaaaaaaatgatataacagatctatatctatgtatgtaacaacacaatcagatgagagaagtgatgttttcagatctgtatacatacaaacacaaacagatgaacaattgatacgtagatccatatgtatacgtacaattagctacacgattaaatgaaaaaaatcaacgatttcggattggtacacacaaacgcaacaatatgaagaaattcatatctgattagatataaacataaccacgtgtagatacacagtcaaatcaacaaatttatagcttctaaacggatgagatgaacaagataaagatattcacataaggcatacataagataagcagattaacaaactagcaataatacatacctaattaaaacaaggaataacagagagagagagagagagagagatttaccttgaaaatgaagaggagaagagaggatttcttaaaattgggggtagagaaagaaagatgatgaattgtgagaaaggagagatagaagggggggttgtatatataggctgtagaagattatttttgtgtttgaggcggtgaaggaagaggggatctgactatgacacgtttgcggttacgtatttcgataggagtctttcaacgcttaacgccgttactctatatgaccgtttgggccgtaacggggccgtttgttaacgctgatgttgattcttttctttctttctttcttccttttttaaagaagcaattgtacaatcgttgctagctgtcaaacggataattcggatacggatatgcctatattcatatccgtaatttttctgcaggaattcgatctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcgaattcggcgcgccatggctcctaagaagaagaggaaggttagcaagctcgagaagtttaccaactgctacagcctctctaagaccctcaggttcaaggctatccctgtgggaaagacccaagagaatatcgacaacaagaggctcctcgtcgaggatgagaagagagctgaagattacaagggcgtgaagaagctcctcgacaggtactacctcagcttcatcaacgatgtgctccacagcatcaagctcaagaacctcaacaactacatcagcctcttccgtaagaaaaccaggaccgagaaagagaacaaagagcttgagaacctcgagatcaacctccgtaaagagatcgccaaggctttcaagggaaacgagggatacaagagcctcttcaagaaggatattatcgagacaatcctgcctgagttcctggacgataaggatgagatcgctctcgtgaacagcttcaacggattcactactgccttcaccggattcttcgacaacagggaaaacatgttcagcgaagaggccaagagcacctctatcgctttcagatgcatcaacgagaacctcacgcgttacatcagcaacatggacatcttcgagaaggtggacgccatcttcgataagcacgaggtgcaagaaatcaaagagaagatcctcaacagcgactacgacgtcgaggacttttttgaaggggagttcttcaacttcgttctcacccaagagggcatcgacgtgtacaacgctattatcggaggattcgtgaccgagtctggggagaagattaagggactcaacgagtacatcaacctgtacaaccagaaaacgaagcagaagctcccgaagttcaagccgctctacaagcaggttctctctgatcgtgagagcctctcattttacggtgagggttacacctctgacgaggaagtgcttgaggttttccgtaacaccctcaacaagaacagcgagatcttctcgtccatcaagaagttggagaaacttttcaagaacttcgacgagtacagcagcgctgggatcttcgttaagaacggacctgctatcagcaccatcagcaaggatattttcggcgagtggaacgtgatcagggacaagtggaatgctgagtacgatgacatccacctcaagaagaaggctgtcgtcactgagaagtacgaggatgacaggcgtaagtcgttcaagaagatcggctctttcagcctcgagcagcttcaagaatacgctgatgctgatctcagcgtggtcgagaagctcaaagagatcatcatccagaaggtcgacgagatctacaaggtgtacgggtcctctgagaagttgttcgatgctgatttcgtcctcgagaagagtctgaagaagaacgacgctgtcgtcgcgatcatgaaggatttgctcgacagcgtgaagtccttcgagaactatatcaaggccttcttcggagagggcaaagagactaatagggacgagtctttctacggggatttcgtgctcgcttacgatatcctcctcaaggtggaccatatctacgacgccatcagaaactacgtgacccagaagccttacagcaaggacaagttcaagttgtactttcagaacccgcagttcatgggcggatgggacaaagacaaagagacagattacagggccaccatcctcaggtacgggtctaagtactacctggccatcatggacaagaaatacgccaagtgcctccaaaagatcgacaaggatgacgtgaacgggaactatgagaagatcaactacaagctccttccgggaccgaacaagatgcttcctaaggtgttcttcagcaagaaatggatggcctactacaacccgtctgaggacatccagaaaatctacaagaacgggaccttcaagaaaggcgacatgttcaacctcaacgactgccacaagctcatcgatttcttcaaggacagcatctcgcgttacccgaagtggtctaacgcttacgactttaacttcagcgagacagaaaagtacaaggatatcgccgggttctaccgtgaggttgaggaacagggttacaaggttagcttcgagagcgcctccaagaaagaggttgacaagttggtcgaagagggcaagctctacatgttccagatctataacaaggacttctccgacaagagccacggaactcctaacctccatacgatgtacttcaagctgcttttcgacgagaacaaccacgggcagatcagactttctggtggtgctgaactcttcatgcgtagggcctcactcaagaaagaagagttggttgttcacccggccaactctccaatcgctaacaagaatcctgacaacccgaaaaagaccaccacgctgtcttacgacgtctacaaggacaaaaggttcagcgaggaccagtacgagcttcatatcccgatcgctatcaacaagtgcccgaagaacatcttcaagatcaataccgaggtgagggtgctgctcaagcacgatgataacccttacgtgatcggaatcgatcgtggtgagagaaacctcctctacatcgttgtggtggacggaaagggaaacatcgtcgagcagtacagcctgaacgagattatcaacaatttcaacggcatcaggatcaagaccgactaccactcactcctcgataagaaagaaaaagagcgtttcgaggccaggcagaactggacttctatcgaaaacatcaaagagttgaaggccggctacatctctcaggtggtgcataagatctgcgagctggtggaaaagtacgatgctgtgatcgctcttgaggacctcaactctgggttcaagaacagtagagtgaaggttgagaagcaggtctaccaaaagttcgagaagatgctcatcgacaagctcaactacatggtggacaaaaagagcaacccttgcgctaccggtggtgctcttaagggataccagatcacgaacaagttcgagtccttcaagagcatgagcacccagaacggcttcatcttctatatccctgcttggctcaccagcaagatcgatccttctactggtttcgtgaacctgctcaagaccaagtacacctcgatcgccgacagcaagaagttcatctcgtctttcgacaggatcatgtacgtgccggaagaggatcttttcgagttcgctctcgactataagaacttcagcaggaccgacgccgactacattaagaagtggaagctctactcctacgggaaccgtatcaggatcttccgaaatccgaagaaaaacaacgtgttcgactgggaagaagtgtgcctcacctctgcctacaaagaactgttcaacaagtacggcatcaactaccagcagggtgatatcagggctcttttgtgcgagcagagcgacaaggcattctacagctcattcatggccctcatgtctctcatgctccagatgaggaactctatcaccggaaggaccgatgtggacttccttatctctccggtcaagaactctgacgggatcttctacgacagccgtaactatgaggctcaagagaacgctatcctgccgaagaatgctgatgcaaacggggcttacaacattgcgagaaaggttctctgggctatcgggcagtttaagaaagcggaagatgagaagctcgacaaggtgaagatcgccatctccaacaaagagtggcttgagtacgctcagacctccgttaagcacaagaggcctgctgctactaagaaagctggccaggccaaaaagaagaagtgaggcgcgccgagctccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggcttgagctccctaggatcaagctttccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctgaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcactcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaagaaataggggttctgcgcacatttccccgaaaagtgccacctgaaattgtaaacgttaatgttttgttaaatttcgcgttaaatatttgttaaatcagcttattttttaaccagtaagcagaaaatgacaaaaatccttataaatcaaaagaatagaccgagttagttgtgagtgttgttccagtttggaacaagagtccactattaaagaacgtggactccaacgtaaaaccgtctatcagggcgatggcccactacgtgaaccatcacccaaatcaagtttttggaggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctactgggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattatcgattaaattaaccctcactaaagggagagacgaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagctgaaccctcga

SEQ ID NO: 69 - DNA, 12928 bp, Artificial Sequence
  pUbitDTnptII_LbCpf1_ crRNA _ ALS_In2 vector
gatcctctcaacacaacatatacaaaacaaacgaatctcaagcaatcaagcattctacttctattgcagcaatttaaatcatttcttttaaagcaaaagcaattttctgaaaattttcaccatttacgaacgatagggcgcgatcccgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtctagaggtaccggttgttggcccatgggagcttggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggacccaagctaggccatcgatacgcgtacgtcgcgaccgcggacatgtacagagctcgagaagtactagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcaagcttatcgatgtcgacaggccttaagggccagatcccccgggctgcagaagcctatactgtacttaacttgattgcataattacttgatcatagactcatagtaaacttgattacacagataagtgaagaaacaaaccaattcaagacataaccaaagagaggtgaaagactgttttatatgtctaacattgcaccttaatatcacactgttagttcctttcttacttaaattcaacccattaaagtaaaaacaacagataataataatttgagaatgaacaaaaggaccatatcatttattaactcttatccatccatttgcattttgatgtccgaaaacaaaaactgaaagaacacagtaaattacaagcagaacaaatgatagaagaaaacagcttttccaatgccataatactcaaacttagtaggattctggtgtgtgggcaatgaaactgatgcattgaacttgacgaacgttgtcgaaaccgatgatacggacgaaagctgggaggcctggtcccattcgccatgccgaagcatgttgcccagccggcgccagcgaggaggctgggaccatgccggccgatatactagttggcttggtgcatatctacacttagtagaaattaatctacacttagtagaaattagacgagcttactcgtttcgtcctcacggactcatcagtaatttggatccgctgcacatacataacatatcaagatcagaacacacatatacacacacaaatacaatcaagtcaacaactccaaaaagtccagatctacatatatacatacgtaaataacaaaatcatgtaaataatcacaatcatgtaatccagatctatgcacatatatatatacacaattaataaaaaaaatgatataacagatctatatctatgtatgtaacaacacaatcagatgagagaagtgatgttttcagatctgtatacatacaaacacaaacagatgaacaattgatacgtagatccatatgtatacgtacaattagctacacgattaaatgaaaaaaatcaacgatttcggattggtacacacaaacgcaacaatatgaagaaattcatatctgattagatataaacataaccacgtgtagatacacagtcaaatcaacaaatttatagcttctaaacggatgagatgaacaagataaagatattcacataaggcatacataagataagcagattaacaaactagcaataatacatacctaattaaaacaaggaataacagagagagagagagagagagagatttaccttgaaaatgaagaggagaagagaggatttcttaaaattgggggtagagaaagaaagatgatgaattgtgagaaaggagagatagaagggggggttgtatatataggctgtagaagattatttttgtgtttgaggcggtgaaggaagaggggatctgactatgacacgtttgcggttacgtatttcgataggagtctttcaacgcttaacgccgttactctatatgaccgtttgggccgtaacggggccgtttgttaacgctgatgttgattcttttctttctttctttcttccttttttaaagaagcaattgtacaatcgttgctagctgtcaaacggataattcggatacggatatgcctatattcatatccgtaatttttctgcaggaattcgatctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcgaattcggcgcgccatggctcctaagaagaagaggaaggttagcaagctcgagaagtttaccaactgctacagcctctctaagaccctcaggttcaaggctatccctgtgggaaagacccaagagaatatcgacaacaagaggctcctcgtcgaggatgagaagagagctgaagattacaagggcgtgaagaagctcctcgacaggtactacctcagcttcatcaacgatgtgctccacagcatcaagctcaagaacctcaacaactacatcagcctcttccgtaagaaaaccaggaccgagaaagagaacaaagagcttgagaacctcgagatcaacctccgtaaagagatcgccaaggctttcaagggaaacgagggatacaagagcctcttcaagaaggatattatcgagacaatcctgcctgagttcctggacgataaggatgagatcgctctcgtgaacagcttcaacggattcactactgccttcaccggattcttcgacaacagggaaaacatgttcagcgaagaggccaagagcacctctatcgctttcagatgcatcaacgagaacctcacgcgttacatcagcaacatggacatcttcgagaaggtggacgccatcttcgataagcacgaggtgcaagaaatcaaagagaagatcctcaacagcgactacgacgtcgaggacttttttgaaggggagttcttcaacttcgttctcacccaagagggcatcgacgtgtacaacgctattatcggaggattcgtgaccgagtctggggagaagattaagggactcaacgagtacatcaacctgtacaaccagaaaacgaagcagaagctcccgaagttcaagccgctctacaagcaggttctctctgatcgtgagagcctctcattttacggtgagggttacacctctgacgaggaagtgcttgaggttttccgtaacaccctcaacaagaacagcgagatcttctcgtccatcaagaagttggagaaacttttcaagaacttcgacgagtacagcagcgctgggatcttcgttaagaacggacctgctatcagcaccatcagcaaggatattttcggcgagtggaacgtgatcagggacaagtggaatgctgagtacgatgacatccacctcaagaagaaggctgtcgtcactgagaagtacgaggatgacaggcgtaagtcgttcaagaagatcggctctttcagcctcgagcagcttcaagaatacgctgatgctgatctcagcgtggtcgagaagctcaaagagatcatcatccagaaggtcgacgagatctacaaggtgtacgggtcctctgagaagttgttcgatgctgatttcgtcctcgagaagagtctgaagaagaacgacgctgtcgtcgcgatcatgaaggatttgctcgacagcgtgaagtccttcgagaactatatcaaggccttcttcggagagggcaaagagactaatagggacgagtctttctacggggatttcgtgctcgcttacgatatcctcctcaaggtggaccatatctacgacgccatcagaaactacgtgacccagaagccttacagcaaggacaagttcaagttgtactttcagaacccgcagttcatgggcggatgggacaaagacaaagagacagattacagggccaccatcctcaggtacgggtctaagtactacctggccatcatggacaagaaatacgccaagtgcctccaaaagatcgacaaggatgacgtgaacgggaactatgagaagatcaactacaagctccttccgggaccgaacaagatgcttcctaaggtgttcttcagcaagaaatggatggcctactacaacccgtctgaggacatccagaaaatctacaagaacgggaccttcaagaaaggcgacatgttcaacctcaacgactgccacaagctcatcgatttcttcaaggacagcatctcgcgttacccgaagtggtctaacgcttacgactttaacttcagcgagacagaaaagtacaaggatatcgccgggttctaccgtgaggttgaggaacagggttacaaggttagcttcgagagcgcctccaagaaagaggttgacaagttggtcgaagagggcaagctctacatgttccagatctataacaaggacttctccgacaagagccacggaactcctaacctccatacgatgtacttcaagctgcttttcgacgagaacaaccacgggcagatcagactttctggtggtgctgaactcttcatgcgtagggcctcactcaagaaagaagagttggttgttcacccggccaactctccaatcgctaacaagaatcctgacaacccgaaaaagaccaccacgctgtcttacgacgtctacaaggacaaaaggttcagcgaggaccagtacgagcttcatatcccgatcgctatcaacaagtgcccgaagaacatcttcaagatcaataccgaggtgagggtgctgctcaagcacgatgataacccttacgtgatcggaatcgatcgtggtgagagaaacctcctctacatcgttgtggtggacggaaagggaaacatcgtcgagcagtacagcctgaacgagattatcaacaatttcaacggcatcaggatcaagaccgactaccactcactcctcgataagaaagaaaaagagcgtttcgaggccaggcagaactggacttctatcgaaaacatcaaagagttgaaggccggctacatctctcaggtggtgcataagatctgcgagctggtggaaaagtacgatgctgtgatcgctcttgaggacctcaactctgggttcaagaacagtagagtgaaggttgagaagcaggtctaccaaaagttcgagaagatgctcatcgacaagctcaactacatggtggacaaaaagagcaacccttgcgctaccggtggtgctcttaagggataccagatcacgaacaagttcgagtccttcaagagcatgagcacccagaacggcttcatcttctatatccctgcttggctcaccagcaagatcgatccttctactggtttcgtgaacctgctcaagaccaagtacacctcgatcgccgacagcaagaagttcatctcgtctttcgacaggatcatgtacgtgccggaagaggatcttttcgagttcgctctcgactataagaacttcagcaggaccgacgccgactacattaagaagtggaagctctactcctacgggaaccgtatcaggatcttccgaaatccgaagaaaaacaacgtgttcgactgggaagaagtgtgcctcacctctgcctacaaagaactgttcaacaagtacggcatcaactaccagcagggtgatatcagggctcttttgtgcgagcagagcgacaaggcattctacagctcattcatggccctcatgtctctcatgctccagatgaggaactctatcaccggaaggaccgatgtggacttccttatctctccggtcaagaactctgacgggatcttctacgacagccgtaactatgaggctcaagagaacgctatcctgccgaagaatgctgatgcaaacggggcttacaacattgcgagaaaggttctctgggctatcgggcagtttaagaaagcggaagatgagaagctcgacaaggtgaagatcgccatctccaacaaagagtggcttgagtacgctcagacctccgttaagcacaagaggcctgctgctactaagaaagctggccaggccaaaaagaagaagtgaggcgcgccgagctccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggcttgagctccctaggatcaagctttccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctgaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcactcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaagaaataggggttctgcgcacatttccccgaaaagtgccacctgaaattgtaaacgttaatgttttgttaaatttcgcgttaaatatttgttaaatcagcttattttttaaccagtaagcagaaaatgacaaaaatccttataaatcaaaagaatagaccgagttagttgtgagtgttgttccagtttggaacaagagtccactattaaagaacgtggactccaacgtaaaaccgtctatcagggcgatggcccactacgtgaaccatcacccaaatcaagtttttggaggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctactgggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattatcgattaaattaaccctcactaaagggagagacgaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagctgaaccctcga

SEQ ID NO: 70 - DNA, 12928 bp, Artificial Sequence; identical to SEQ ID NO: 69
  pUbitDTnptII_LbCpf1_ crRNA _ Cyp_Del

SEQ ID NO: 71 - DNA, 16875 bp, Artificial Sequence
  pZFNnptII_LbCpf1_crRNA_ALS_G/T vector
ccggattgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgaagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccattttctgtgattttggatgtgtttttgtatggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcattccggactgtggggccatatcccagaactggttgagtcggtccaacacctgggtgccaatcatgtcgatggtggggtatggccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagaggccatggcggccgcactagtggatcccccgggatgcatgaattcaagcttatcgatgtcgacaggccttaagggccagatcccccgggctgcagaagcctatactgtacttaacttgattgcataattacttgatcatagactcatagtaaacttgattacacagataagtgaagaaacaaaccaattcaagacataaccaaagagaggtgaaagactgttttatatgtctaacattgcaccttaatatcacactgttagttcctttcttacttaaattcaacccattaaagtaaaaacaacagataataataatttgagaatgaacaaaaggaccatatcatttattaactcttatccatccatttgcattttgatgtccgaaaacaaaaactgaaagaacacagtaaattacaagcagaacaaatgatagaagaaaacagcttttccaatgccataatactcaaacttagtaggattctggtgtgtgggcaatgaaactgatgcattgaacttgacgaacgttgtcgaaaccgatgatacggacgaaagctgggaggcctggtcccattcgccatgccgaagcatgttgcccagccggcgccagcgaggaggctgggaccatgccggcctagaagggtaacctgttggtatggatctacacttagtagaaattaatctacacttagtagaaattagacgagcttactcgtttcgtcctcacggactcatcagtaatttggatccgctgcacatacataacatatcaagatcagaacacacatatacacacacaaatacaatcaagtcaacaactccaaaaagtccagatctacatatatacatacgtaaataacaaaatcatgtaaataatcacaatcatgtaatccagatctatgcacatatatatatacacaattaataaaaaaaatgatataacagatctatatctatgtatgtaacaacacaatcagatgagagaagtgatgttttcagatctgtatacatacaaacacaaacagatgaacaattgatacgtagatccatatgtatacgtacaattagctacacgattaaatgaaaaaaatcaacgatttcggattggtacacacaaacgcaacaatatgaagaaattcatatctgattagatataaacataaccacgtgtagatacacagtcaaatcaacaaatttatagcttctaaacggatgagatgaacaagataaagatattcacataaggcatacataagataagcagattaacaaactagcaataatacatacctaattaaaacaaggaataacagagagagagagagagagagagatttaccttgaaaatgaagaggagaagagaggatttcttaaaattgggggtagagaaagaaagatgatgaattgtgagaaaggagagatagaagggggggttgtatatataggctgtagaagattatttttgtgtttgaggcggtgaaggaagaggggatctgactatgacacgtttgcggttacgtatttcgataggagtctttcaacgcttaacgccgttactctatatgaccgtttgggccgtaacggggccgtttgttaacgctgatgttgattcttttctttctttctttcttccttttttaaagaagcaattgtacaatcgttgctagctgtcaaacggataattcggatacggatatgcctatattcatatccgtaatttttctgcaggaattcgatctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcgaattcggcgcgccatggctcctaagaagaagaggaaggttagcaagctcgagaagtttaccaactgctacagcctctctaagaccctcaggttcaaggctatccctgtgggaaagacccaagagaatatcgacaacaagaggctcctcgtcgaggatgagaagagagctgaagattacaagggcgtgaagaagctcctcgacaggtactacctcagcttcatcaacgatgtgctccacagcatcaagctcaagaacctcaacaactacatcagcctcttccgtaagaaaaccaggaccgagaaagagaacaaagagcttgagaacctcgagatcaacctccgtaaagagatcgccaaggctttcaagggaaacgagggatacaagagcctcttcaagaaggatattatcgagacaatcctgcctgagttcctggacgataaggatgagatcgctctcgtgaacagcttcaacggattcactactgccttcaccggattcttcgacaacagggaaaacatgttcagcgaagaggccaagagcacctctatcgctttcagatgcatcaacgagaacctcacgcgttacatcagcaacatggacatcttcgagaaggtggacgccatcttcgataagcacgaggtgcaagaaatcaaagagaagatcctcaacagcgactacgacgtcgaggacttttttgaaggggagttcttcaacttcgttctcacccaagagggcatcgacgtgtacaacgctattatcggaggattcgtgaccgagtctggggagaagattaagggactcaacgagtacatcaacctgtacaaccagaaaacgaagcagaagctcccgaagttcaagccgctctacaagcaggttctctctgatcgtgagagcctctcattttacggtgagggttacacctctgacgaggaagtgcttgaggttttccgtaacaccctcaacaagaacagcgagatcttctcgtccatcaagaagttggagaaacttttcaagaacttcgacgagtacagcagcgctgggatcttcgttaagaacggacctgctatcagcaccatcagcaaggatattttcggcgagtggaacgtgatcagggacaagtggaatgctgagtacgatgacatccacctcaagaagaaggctgtcgtcactgagaagtacgaggatgacaggcgtaagtcgttcaagaagatcggctctttcagcctcgagcagcttcaagaatacgctgatgctgatctcagcgtggtcgagaagctcaaagagatcatcatccagaaggtcgacgagatctacaaggtgtacgggtcctctgagaagttgttcgatgctgatttcgtcctcgagaagagtctgaagaagaacgacgctgtcgtcgcgatcatgaaggatttgctcgacagcgtgaagtccttcgagaactatatcaaggccttcttcggagagggcaaagagactaatagggacgagtctttctacggggatttcgtgctcgcttacgatatcctcctcaaggtggaccatatctacgacgccatcagaaactacgtgacccagaagccttacagcaaggacaagttcaagttgtactttcagaacccgcagttcatgggcggatgggacaaagacaaagagacagattacagggccaccatcctcaggtacgggtctaagtactacctggccatcatggacaagaaatacgccaagtgcctccaaaagatcgacaaggatgacgtgaacgggaactatgagaagatcaactacaagctccttccgggaccgaacaagatgcttcctaaggtgttcttcagcaagaaatggatggcctactacaacccgtctgaggacatccagaaaatctacaagaacgggaccttcaagaaaggcgacatgttcaacctcaacgactgccacaagctcatcgatttcttcaaggacagcatctcgcgttacccgaagtggtctaacgcttacgactttaacttcagcgagacagaaaagtacaaggatatcgccgggttctaccgtgaggttgaggaacagggttacaaggttagcttcgagagcgcctccaagaaagaggttgacaagttggtcgaagagggcaagctctacatgttccagatctataacaaggacttctccgacaagagccacggaactcctaacctccatacgatgtacttcaagctgcttttcgacgagaacaaccacgggcagatcagactttctggtggtgctgaactcttcatgcgtagggcctcactcaagaaagaagagttggttgttcacccggccaactctccaatcgctaacaagaatcctgacaacccgaaaaagaccaccacgctgtcttacgacgtctacaaggacaaaaggttcagcgaggaccagtacgagcttcatatcccgatcgctatcaacaagtgcccgaagaacatcttcaagatcaataccgaggtgagggtgctgctcaagcacgatgataacccttacgtgatcggaatcgatcgtggtgagagaaacctcctctacatcgttgtggtggacggaaagggaaacatcgtcgagcagtacagcctgaacgagattatcaacaatttcaacggcatcaggatcaagaccgactaccactcactcctcgataagaaagaaaaagagcgtttcgaggccaggcagaactggacttctatcgaaaacatcaaagagttgaaggccggctacatctctcaggtggtgcataagatctgcgagctggtggaaaagtacgatgctgtgatcgctcttgaggacctcaactctgggttcaagaacagtagagtgaaggttgagaagcaggtctaccaaaagttcgagaagatgctcatcgacaagctcaactacatggtggacaaaaagagcaacccttgcgctaccggtggtgctcttaagggataccagatcacgaacaagttcgagtccttcaagagcatgagcacccagaacggcttcatcttctatatccctgcttggctcaccagcaagatcgatccttctactggtttcgtgaacctgctcaagaccaagtacacctcgatcgccgacagcaagaagttcatctcgtctttcgacaggatcatgtacgtgccggaagaggatcttttcgagttcgctctcgactataagaacttcagcaggaccgacgccgactacattaagaagtggaagctctactcctacgggaaccgtatcaggatcttccgaaatccgaagaaaaacaacgtgttcgactgggaagaagtgtgcctcacctctgcctacaaagaactgttcaacaagtacggcatcaactaccagcagggtgatatcagggctcttttgtgcgagcagagcgacaaggcattctacagctcattcatggccctcatgtctctcatgctccagatgaggaactctatcaccggaaggaccgatgtggacttccttatctctccggtcaagaactctgacgggatcttctacgacagccgtaactatgaggctcaagagaacgctatcctgccgaagaatgctgatgcaaacggggcttacaacattgcgagaaaggttctctgggctatcgggcagtttaagaaagcggaagatgagaagctcgacaaggtgaagatcgccatctccaacaaagagtggcttgagtacgctcagacctccgttaagcacaagaggcctgctgctactaagaaagctggccaggccaaaaagaagaagtgaggcgcgccgagctccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggcttgagctccctaggatcaagcttatcgatgtcgacaggccttaagggccagatcttgggcccggtacccgatcagattgtcgtttcccgccttcggtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatatcgcggtgcatctcggtcttgcctgggcagtcgccgccgacgccgttgatgtggacgccgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcaggggtacgtcgaggtcgatccaacccctccgctgctatagtgcagtcggcttctgacgttcagtgcagccgtcgtctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcgcgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctcgcggcctggccggcgagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggcgagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccggtgggtatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatagcttgcttggtcgttccgcgtgaacgtcggctcgattgtacctgcgttcaaatactttgcgatcgtgttgcgcgcctgcccggtgcgtcggctgatctcacggatcgactgcttctctcgcaacgccatccgacggatgatgtttaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcggcggcaggatatattcaattgtaaatggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagttcgagcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgtttctatgcttcaattctgctctatagatgttcttctagtcttaacttttagaaggcacaacatttgaaatgttggtaatttttctgctgggtagtgaaccattgtaatttgtgtttgcttatattatccgttgggtttctctatgttatctgtggggccatatcccagaactggttgagtcggtccaacacctgggtgccaatcatgtcgatggtggggtatggt

SEQ ID NO: 72 - DNA, 16875 bp, Artificial Sequence
  pZFNnptII_LbCpf1_crRNA_ALS_In1 vector
ccggattgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgaagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccattttctgtgattttggatgtgtttttgtatggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcattccggactgtggggccatatcccagaactggttgagtcggtccaacacctgggtgccaatcatgtcgatggtggggtatggccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagaggccatggcggccgcactagtggatcccccgggatgcatgaattcaagcttatcgatgtcgacaggccttaagggccagatcccccgggctgcagaagcctatactgtacttaacttgattgcataattacttgatcatagactcatagtaaacttgattacacagataagtgaagaaacaaaccaattcaagacataaccaaagagaggtgaaagactgttttatatgtctaacattgcaccttaatatcacactgttagttcctttcttacttaaattcaacccattaaagtaaaaacaacagataataataatttgagaatgaacaaaaggaccatatcatttattaactcttatccatccatttgcattttgatgtccgaaaacaaaaactgaaagaacacagtaaattacaagcagaacaaatgatagaagaaaacagcttttccaatgccataatactcaaacttagtaggattctggtgtgtgggcaatgaaactgatgcattgaacttgacgaacgttgtcgaaaccgatgatacggacgaaagctgggaggcctggtcccattcgccatgccgaagcatgttgcccagccggcgccagcgaggaggctgggaccatgccggccactacgttgtgttgtaggtatcggatctacacttagtagaaattaatctacacttagtagaaattagacgagcttactcgtttcgtcctcacggactcatcagtaatttggatccgctgcacatacataacatatcaagatcagaacacacatatacacacacaaatacaatcaagtcaacaactccaaaaagtccagatctacatatatacatacgtaaataacaaaatcatgtaaataatcacaatcatgtaatccagatctatgcacatatatatatacacaattaataaaaaaaatgatataacagatctatatctatgtatgtaacaacacaatcagatgagagaagtgatgttttcagatctgtatacatacaaacacaaacagatgaacaattgatacgtagatccatatgtatacgtacaattagctacacgattaaatgaaaaaaatcaacgatttcggattggtacacacaaacgcaacaatatgaagaaattcatatctgattagatataaacataaccacgtgtagatacacagtcaaatcaacaaatttatagcttctaaacggatgagatgaacaagataaagatattcacataaggcatacataagataagcagattaacaaactagcaataatacatacctaattaaaacaaggaataacagagagagagagagagagagagatttaccttgaaaatgaagaggagaagagaggatttcttaaaattgggggtagagaaagaaagatgatgaattgtgagaaaggagagatagaagggggggttgtatatataggctgtagaagattatttttgtgtttgaggcggtgaaggaagaggggatctgactatgacacgtttgcggttacgtatttcgataggagtctttcaacgcttaacgccgttactctatatgaccgtttgggccgtaacggggccgtttgttaacgctgatgttgattcttttctttctttctttcttccttttttaaagaagcaattgtacaatcgttgctagctgtcaaacggataattcggatacggatatgcctatattcatatccgtaatttttctgcaggaattcgatctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcgaattcggcgcgccatggctcctaagaagaagaggaaggttagcaagctcgagaagtttaccaactgctacagcctctctaagaccctcaggttcaaggctatccctgtgggaaagacccaagagaatatcgacaacaagaggctcctcgtcgaggatgagaagagagctgaagattacaagggcgtgaagaagctcctcgacaggtactacctcagcttcatcaacgatgtgctccacagcatcaagctcaagaacctcaacaactacatcagcctcttccgtaagaaaaccaggaccgagaaagagaacaaagagcttgagaacctcgagatcaacctccgtaaagagatcgccaaggctttcaagggaaacgagggatacaagagcctcttcaagaaggatattatcgagacaatcctgcctgagttcctggacgataaggatgagatcgctctcgtgaacagcttcaacggattcactactgccttcaccggattcttcgacaacagggaaaacatgttcagcgaagaggccaagagcacctctatcgctttcagatgcatcaacgagaacctcacgcgttacatcagcaacatggacatcttcgagaaggtggacgccatcttcgataagcacgaggtgcaagaaatcaaagagaagatcctcaacagcgactacgacgtcgaggacttttttgaaggggagttcttcaacttcgttctcacccaagagggcatcgacgtgtacaacgctattatcggaggattcgtgaccgagtctggggagaagattaagggactcaacgagtacatcaacctgtacaaccagaaaacgaagcagaagctcccgaagttcaagccgctctacaagcaggttctctctgatcgtgagagcctctcattttacggtgagggttacacctctgacgaggaagtgcttgaggttttccgtaacaccctcaacaagaacagcgagatcttctcgtccatcaagaagttggagaaacttttcaagaacttcgacgagtacagcagcgctgggatcttcgttaagaacggacctgctatcagcaccatcagcaaggatattttcggcgagtggaacgtgatcagggacaagtggaatgctgagtacgatgacatccacctcaagaagaaggctgtcgtcactgagaagtacgaggatgacaggcgtaagtcgttcaagaagatcggctctttcagcctcgagcagcttcaagaatacgctgatgctgatctcagcgtggtcgagaagctcaaagagatcatcatccagaaggtcgacgagatctacaaggtgtacgggtcctctgagaagttgttcgatgctgatttcgtcctcgagaagagtctgaagaagaacgacgctgtcgtcgcgatcatgaaggatttgctcgacagcgtgaagtccttcgagaactatatcaaggccttcttcggagagggcaaagagactaatagggacgagtctttctacggggatttcgtgctcgcttacgatatcctcctcaaggtggaccatatctacgacgccatcagaaactacgtgacccagaagccttacagcaaggacaagttcaagttgtactttcagaacccgcagttcatgggcggatgggacaaagacaaagagacagattacagggccaccatcctcaggtacgggtctaagtactacctggccatcatggacaagaaatacgccaagtgcctccaaaagatcgacaaggatgacgtgaacgggaactatgagaagatcaactacaagctccttccgggaccgaacaagatgcttcctaaggtgttcttcagcaagaaatggatggcctactacaacccgtctgaggacatccagaaaatctacaagaacgggaccttcaagaaaggcgacatgttcaacctcaacgactgccacaagctcatcgatttcttcaaggacagcatctcgcgttacccgaagtggtctaacgcttacgactttaacttcagcgagacagaaaagtacaaggatatcgccgggttctaccgtgaggttgaggaacagggttacaaggttagcttcgagagcgcctccaagaaagaggttgacaagttggtcgaagagggcaagctctacatgttccagatctataacaaggacttctccgacaagagccacggaactcctaacctccatacgatgtacttcaagctgcttttcgacgagaacaaccacgggcagatcagactttctggtggtgctgaactcttcatgcgtagggcctcactcaagaaagaagagttggttgttcacccggccaactctccaatcgctaacaagaatcctgacaacccgaaaaagaccaccacgctgtcttacgacgtctacaaggacaaaaggttcagcgaggaccagtacgagcttcatatcccgatcgctatcaacaagtgcccgaagaacatcttcaagatcaataccgaggtgagggtgctgctcaagcacgatgataacccttacgtgatcggaatcgatcgtggtgagagaaacctcctctacatcgttgtggtggacggaaagggaaacatcgtcgagcagtacagcctgaacgagattatcaacaatttcaacggcatcaggatcaagaccgactaccactcactcctcgataagaaagaaaaagagcgtttcgaggccaggcagaactggacttctatcgaaaacatcaaagagttgaaggccggctacatctctcaggtggtgcataagatctgcgagctggtggaaaagtacgatgctgtgatcgctcttgaggacctcaactctgggttcaagaacagtagagtgaaggttgagaagcaggtctaccaaaagttcgagaagatgctcatcgacaagctcaactacatggtggacaaaaagagcaacccttgcgctaccggtggtgctcttaagggataccagatcacgaacaagttcgagtccttcaagagcatgagcacccagaacggcttcatcttctatatccctgcttggctcaccagcaagatcgatccttctactggtttcgtgaacctgctcaagaccaagtacacctcgatcgccgacagcaagaagttcatctcgtctttcgacaggatcatgtacgtgccggaagaggatcttttcgagttcgctctcgactataagaacttcagcaggaccgacgccgactacattaagaagtggaagctctactcctacgggaaccgtatcaggatcttccgaaatccgaagaaaaacaacgtgttcgactgggaagaagtgtgcctcacctctgcctacaaagaactgttcaacaagtacggcatcaactaccagcagggtgatatcagggctcttttgtgcgagcagagcgacaaggcattctacagctcattcatggccctcatgtctctcatgctccagatgaggaactctatcaccggaaggaccgatgtggacttccttatctctccggtcaagaactctgacgggatcttctacgacagccgtaactatgaggctcaagagaacgctatcctgccgaagaatgctgatgcaaacggggcttacaacattgcgagaaaggttctctgggctatcgggcagtttaagaaagcggaagatgagaagctcgacaaggtgaagatcgccatctccaacaaagagtggcttgagtacgctcagacctccgttaagcacaagaggcctgctgctactaagaaagctggccaggccaaaaagaagaagtgaggcgcgccgagctccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggcttgagctccctaggatcaagcttatcgatgtcgacaggccttaagggccagatcttgggcccggtacccgatcagattgtcgtttcccgccttcggtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatatcgcggtgcatctcggtcttgcctgggcagtcgccgccgacgccgttgatgtggacgccgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcaggggtacgtcgaggtcgatccaacccctccgctgctatagtgcagtcggcttctgacgttcagtgcagccgtcgtctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcgcgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctcgcggcctggccggcgagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggcgagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccggtgggtatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatagcttgcttggtcgttccgcgtgaacgtcggctcgattgtacctgcgttcaaatactttgcgatcgtgttgcgcgcctgcccggtgcgtcggctgatctcacggatcgactgcttctctcgcaacgccatccgacggatgatgtttaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcggcggcaggatatattcaattgtaaatggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagttcgagcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgtttctatgcttcaattctgctctatagatgttcttctagtcttaacttttagaaggcacaacatttgaaatgttggtaatttttctgctgggtagtgaaccattgtaatttgtgtttgcttatattatccgttgggtttctctatgttatctgtggggccatatcccagaactggttgagtcggtccaacacctgggtgccaatcatgtcgatggtggggtatggt

SEQ ID NO: 73 - DNA, 16875 bp, Artificial Sequence
  pZFNnptII_LbCpf1_crRNA_ALS_In2 vector
ccggattgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgaagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccattttctgtgattttggatgtgtttttgtatggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcattccggactgtggggccatatcccagaactggttgagtcggtccaacacctgggtgccaatcatgtcgatggtggggtatggccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagaggccatggcggccgcactagtggatcccccgggatgcatgaattcaagcttatcgatgtcgacaggccttaagggccagatcccccgggctgcagaagcctatactgtacttaacttgattgcataattacttgatcatagactcatagtaaacttgattacacagataagtgaagaaacaaaccaattcaagacataaccaaagagaggtgaaagactgttttatatgtctaacattgcaccttaatatcacactgttagttcctttcttacttaaattcaacccattaaagtaaaaacaacagataataataatttgagaatgaacaaaaggaccatatcatttattaactcttatccatccatttgcattttgatgtccgaaaacaaaaactgaaagaacacagtaaattacaagcagaacaaatgatagaagaaaacagcttttccaatgccataatactcaaacttagtaggattctggtgtgtgggcaatgaaactgatgcattgaacttgacgaacgttgtcgaaaccgatgatacggacgaaagctgggaggcctggtcccattcgccatgccgaagcatgttgcccagccggcgccagcgaggaggctgggaccatgccggccgatatactagttggcttggtgcatatctacacttagtagaaattaatctacacttagtagaaattagacgagcttactcgtttcgtcctcacggactcatcagtaatttggatccgctgcacatacataacatatcaagatcagaacacacatatacacacacaaatacaatcaagtcaacaactccaaaaagtccagatctacatatatacatacgtaaataacaaaatcatgtaaataatcacaatcatgtaatccagatctatgcacatatatatatacacaattaataaaaaaaatgatataacagatctatatctatgtatgtaacaacacaatcagatgagagaagtgatgttttcagatctgtatacatacaaacacaaacagatgaacaattgatacgtagatccatatgtatacgtacaattagctacacgattaaatgaaaaaaatcaacgatttcggattggtacacacaaacgcaacaatatgaagaaattcatatctgattagatataaacataaccacgtgtagatacacagtcaaatcaacaaatttatagcttctaaacggatgagatgaacaagataaagatattcacataaggcatacataagataagcagattaacaaactagcaataatacatacctaattaaaacaaggaataacagagagagagagagagagagagatttaccttgaaaatgaagaggagaagagaggatttcttaaaattgggggtagagaaagaaagatgatgaattgtgagaaaggagagatagaagggggggttgtatatataggctgtagaagattatttttgtgtttgaggcggtgaaggaagaggggatctgactatgacacgtttgcggttacgtatttcgataggagtctttcaacgcttaacgccgttactctatatgaccgtttgggccgtaacggggccgtttgttaacgctgatgttgattcttttctttctttctttcttccttttttaaagaagcaattgtacaatcgttgctagctgtcaaacggataattcggatacggatatgcctatattcatatccgtaatttttctgcaggaattcgatctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcgaattcggcgcgccatggctcctaagaagaagaggaaggttagcaagctcgagaagtttaccaactgctacagcctctctaagaccctcaggttcaaggctatccctgtgggaaagacccaagagaatatcgacaacaagaggctcctcgtcgaggatgagaagagagctgaagattacaagggcgtgaagaagctcctcgacaggtactacctcagcttcatcaacgatgtgctccacagcatcaagctcaagaacctcaacaactacatcagcctcttccgtaagaaaaccaggaccgagaaagagaacaaagagcttgagaacctcgagatcaacctccgtaaagagatcgccaaggctttcaagggaaacgagggatacaagagcctcttcaagaaggatattatcgagacaatcctgcctgagttcctggacgataaggatgagatcgctctcgtgaacagcttcaacggattcactactgccttcaccggattcttcgacaacagggaaaacatgttcagcgaagaggccaagagcacctctatcgctttcagatgcatcaacgagaacctcacgcgttacatcagcaacatggacatcttcgagaaggtggacgccatcttcgataagcacgaggtgcaagaaatcaaagagaagatcctcaacagcgactacgacgtcgaggacttttttgaaggggagttcttcaacttcgttctcacccaagagggcatcgacgtgtacaacgctattatcggaggattcgtgaccgagtctggggagaagattaagggactcaacgagtacatcaacctgtacaaccagaaaacgaagcagaagctcccgaagttcaagccgctctacaagcaggttctctctgatcgtgagagcctctcattttacggtgagggttacacctctgacgaggaagtgcttgaggttttccgtaacaccctcaacaagaacagcgagatcttctcgtccatcaagaagttggagaaacttttcaagaacttcgacgagtacagcagcgctgggatcttcgttaagaacggacctgctatcagcaccatcagcaaggatattttcggcgagtggaacgtgatcagggacaagtggaatgctgagtacgatgacatccacctcaagaagaaggctgtcgtcactgagaagtacgaggatgacaggcgtaagtcgttcaagaagatcggctctttcagcctcgagcagcttcaagaatacgctgatgctgatctcagcgtggtcgagaagctcaaagagatcatcatccagaaggtcgacgagatctacaaggtgtacgggtcctctgagaagttgttcgatgctgatttcgtcctcgagaagagtctgaagaagaacgacgctgtcgtcgcgatcatgaaggatttgctcgacagcgtgaagtccttcgagaactatatcaaggccttcttcggagagggcaaagagactaatagggacgagtctttctacggggatttcgtgctcgcttacgatatcctcctcaaggtggaccatatctacgacgccatcagaaactacgtgacccagaagccttacagcaaggacaagttcaagttgtactttcagaacccgcagttcatgggcggatgggacaaagacaaagagacagattacagggccaccatcctcaggtacgggtctaagtactacctggccatcatggacaagaaatacgccaagtgcctccaaaagatcgacaaggatgacgtgaacgggaactatgagaagatcaactacaagctccttccgggaccgaacaagatgcttcctaaggtgttcttcagcaagaaatggatggcctactacaacccgtctgaggacatccagaaaatctacaagaacgggaccttcaagaaaggcgacatgttcaacctcaacgactgccacaagctcatcgatttcttcaaggacagcatctcgcgttacccgaagtggtctaacgcttacgactttaacttcagcgagacagaaaagtacaaggatatcgccgggttctaccgtgaggttgaggaacagggttacaaggttagcttcgagagcgcctccaagaaagaggttgacaagttggtcgaagagggcaagctctacatgttccagatctataacaaggacttctccgacaagagccacggaactcctaacctccatacgatgtacttcaagctgcttttcgacgagaacaaccacgggcagatcagactttctggtggtgctgaactcttcatgcgtagggcctcactcaagaaagaagagttggttgttcacccggccaactctccaatcgctaacaagaatcctgacaacccgaaaaagaccaccacgctgtcttacgacgtctacaaggacaaaaggttcagcgaggaccagtacgagcttcatatcccgatcgctatcaacaagtgcccgaagaacatcttcaagatcaataccgaggtgagggtgctgctcaagcacgatgataacccttacgtgatcggaatcgatcgtggtgagagaaacctcctctacatcgttgtggtggacggaaagggaaacatcgtcgagcagtacagcctgaacgagattatcaacaatttcaacggcatcaggatcaagaccgactaccactcactcctcgataagaaagaaaaagagcgtttcgaggccaggcagaactggacttctatcgaaaacatcaaagagttgaaggccggctacatctctcaggtggtgcataagatctgcgagctggtggaaaagtacgatgctgtgatcgctcttgaggacctcaactctgggttcaagaacagtagagtgaaggttgagaagcaggtctaccaaaagttcgagaagatgctcatcgacaagctcaactacatggtggacaaaaagagcaacccttgcgctaccggtggtgctcttaagggataccagatcacgaacaagttcgagtccttcaagagcatgagcacccagaacggcttcatcttctatatccctgcttggctcaccagcaagatcgatccttctactggtttcgtgaacctgctcaagaccaagtacacctcgatcgccgacagcaagaagttcatctcgtctttcgacaggatcatgtacgtgccggaagaggatcttttcgagttcgctctcgactataagaacttcagcaggaccgacgccgactacattaagaagtggaagctctactcctacgggaaccgtatcaggatcttccgaaatccgaagaaaaacaacgtgttcgactgggaagaagtgtgcctcacctctgcctacaaagaactgttcaacaagtacggcatcaactaccagcagggtgatatcagggctcttttgtgcgagcagagcgacaaggcattctacagctcattcatggccctcatgtctctcatgctccagatgaggaactctatcaccggaaggaccgatgtggacttccttatctctccggtcaagaactctgacgggatcttctacgacagccgtaactatgaggctcaagagaacgctatcctgccgaagaatgctgatgcaaacggggcttacaacattgcgagaaaggttctctgggctatcgggcagtttaagaaagcggaagatgagaagctcgacaaggtgaagatcgccatctccaacaaagagtggcttgagtacgctcagacctccgttaagcacaagaggcctgctgctactaagaaagctggccaggccaaaaagaagaagtgaggcgcgccgagctccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggcttgagctccctaggatcaagcttatcgatgtcgacaggccttaagggccagatcttgggcccggtacccgatcagattgtcgtttcccgccttcggtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatatcgcggtgcatctcggtcttgcctgggcagtcgccgccgacgccgttgatgtggacgccgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcaggggtacgtcgaggtcgatccaacccctccgctgctatagtgcagtcggcttctgacgttcagtgcagccgtcgtctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcgcgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctcgcggcctggccggcgagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggcgagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccggtgggtatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatagcttgcttggtcgttccgcgtgaacgtcggctcgattgtacctgcgttcaaatactttgcgatcgtgttgcgcgcctgcccggtgcgtcggctgatctcacggatcgactgcttctctcgcaacgccatccgacggatgatgtttaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcggcggcaggatatattcaattgtaaatggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagttcgagcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgtttctatgcttcaattctgctctatagatgttcttctagtcttaacttttagaaggcacaacatttgaaatgttggtaatttttctgctgggtagtgaaccattgtaatttgtgtttgcttatattatccgttgggtttctctatgttatctgtggggccatatcccagaactggttgagtcggtccaacacctgggtgccaatcatgtcgatggtggggtatggt

SEQ ID NO: 74 - DNA, 16875 bp, Artificial Sequence; identical to SEQ ID NO: 73
  pZFNnptII_LbCpf1_crRNA_Cyp_Del vector

SEQ ID NO: 75 - DNA, 4120 bp, Beta vulgaris
gtatatcgttgccacatgtgcgttgaatttttccttttcctatcctttccactccatattctcctcaaaagtgtgtaaaaatccgacacacgagtagaatgggattgaagtgggtcaagatctgaaaccaatgggtcaatgccacaaaataaggtaaggtttctcgcagtagcaaaaaaataaagttaagttgagagaaaaattatgaatagttgtttctcgtgaagagttgtatacaaaaaaagtctaatttgatacattttcttttacatttataaaggattgaccaatcatccaaattaccaaatatttaggatataaatctttcagattacaacccatatatgatacactaaattttacatgaggcaatggaggatttgcatgaatatcgaggagagaaaaaattagttacaaaacttgcataatttatccaaaccaaatcaagtcaagaaacaacgaacaatattatcattagtactataagtatatattataggcttagagcaaagccctaactaccacactgcacacaaatgataactagtaagagaggaaaatacaaatttaagattcaacatagcaaattattcatgattcatgattcatgattcatgattcatgattcacgaacatcaagaatggtatagctgataaaggacaatttaaacataagtgtaaagctcgcacatcatcaattatattcgcatactactagaccaatctttacttagtacatgtgttagtacatgtgttacttcatatcagatgtattgattgttgccaatgacatatcatgttcacttaatcttagggccatttaattataacatggagaataatacaacttaaaattatgtggtggctatcatctcattttctagataattaaacctttattttgtatacatatatattgtctttacatagcaaaacaatattgaaggtataacaacctttcccttttcttttactacatgtttatgttagagtttttcgatttacgattgtggtaaattaattgtaattgatcggttgtcttgtagtcaagaaatgacgtatgaatcaatttagggcatgttctcttcggcataaaacagctgaactgaattgaactgaactgaaatgaatagtgatatgtgagagtaaaagtattgtcaagagctgaactgagctgaactgaacggatctgaactgaactgatctaatctgaactaatctgaactgaactgaactgaattgaactgaaaataagctagggaaaacagacccttactactattatataacctcgtttaaatattaggaaattaaaaaaataattatatttctttatactttattaacctattgaggtttttatattgactcccaaatactattttatagatcatgccatgttaatgagcaaactactttctcacatctttataggagaaaaagtagatcactcactagcatatcatgaccagcgaaaccaaccaacgctaatagttttattgcttccattagagataagagttaactaataataccatctttgtgaaattttgatggattttggaactttagcaatatatttactgtgtgcactttttgctaccaaaattttataccaatggctcaagtcctacttatacacaacatacaaactccctcccggcccaccaaggtggcccttatttggaaacctccttcaactagggccacttccccaccgcgatttcgcctcattttgtgaaaaatatgggcctttagtctacataaggcttggtaatgtggatgccataaccactaatgatccagaaatcatacgtgagatcttagttcgacaagatgatgtatttgcgtctcgtccacataccttagccgcaacccacttggcttacaatagtggtgatgtggccttggctccaatgggaccaaaatggaaaagaatgagaaggatatgcatggagcacttgctcacaactagacgacttgaactatttgtgagtcatagggctgatgaggcacgacatttggtccaagacgtattaactcgttcccacaaagataaagttgttaatttgagggaagtgttaggtgcattttctatgaataacgtgactagaatgttgctagggaagcaatactttggggccgggacggcgggcccacaagaggctctagagtttatgcatataacacatgagttgttttggttactaggcttgatttacttgggtgattatttgcctttttggaggtgggttgatccatatggatgtgaaaagaaaatgagggaagttgaaaaaagggtagatgatttccatcgcaaaattatagaggaacataggaaggagaagaaaaggaaagaagaaatgggagtgaatgagggtgaaatggattttgtagatattttgttggctttgcctggtgaaaatggaaatgagcatatggatgatgcagatattaaagctctaattcaggtaattcatgtataatttgaatgtgatcgatacaaagtttgatagaaaacatatttgcataaatatatggttgccctactagacccaataaaatacataattattgccttactagttgaaagttgaaacaacctagctaccattttgttgtgattatcattagccaaccaaaattatttcttgcatccatatattaatgttgagatcagagtcggcatatttacaattacttgtaacattttaagcaaacaaattaaaatattttttggcaagtccattttattgaataatacctatatcttaaaatgaattcttggtcatgtacacttgcctttcaaggtaccaatatttgaccatatgtaattactattaacaaatttgataaaatctaataatatgtaaatatacattcacgcacatattagaaacaaagatcacaaatgataatgcaaaataacttatttgaactaatgttgtgaagttaaatttggaacaaaaggtatatttgtattgccgaattttaatttataaattacttataacaacaactcaatatgtaaaactgttaagatggagtgtggatagaatgagatgagtatacttttactagttaccactcgaaaatgcatttcctcctttgtttatagttgttctaacttctattatcataaataattttttggacttatttcaatgtatatttacaacgctaattgtttaattttttaaaaatacataatgtaaacaaggatttcatggtcaattatacacattataaatattatcttaaaaaacttattaatgctcaattagtatccataatataggatatgatagcagcagcaacagacacatcagctgtaaccaacgaatgggccatggcagaagtaataaaacacccacgtgtcctccacaagatccaacaagagcttaacacaatagtaggacccaatcgaatggtaacagaatcagatcttccccaccttaactacctacgttgtgtcgtacgtgaaacgttccgaatgcatccagcaggaccctttttaatcccacatgaatcactacgccatacaacaatcaacggctatgatatcccatctgggacacgtgtcttcatcaacacacatgggttaggacgtaaccttaaagtgtgggacaacatagaggatttttaccctgaaagacattggccgttggatggaagtagagttgagattagccatggatctgattttaaaatattaccatttagtgctgggaagagaagatgtcctggggccccacttggggtggtgtttgtgttgatgggattggctacactttttcatgcatttgattggttaccacctgatggaatgaaggcagaagaaattgatactaaggaagtttatgggatgactatgcctaaagctcaacctttaatggctttggctaaacctaggcttgctcatttatatctttgatacatgttcatattgtggtgcacttataagcacaatagacaaatacaagtttgtatcgactctaacatgttgtttagtattagtatactgcaactctacaagtatgtaatttctataaactataaacacaagtcataacgcattttgttttgaaaaaaaagaggttacattgtcttacaccataaa

SEQ ID NO: 76 - DNA, 4220 bp, Beta vulgaris
gtatatcgttgccacatgtgcgttgaatttttccttttcctatcctttccactccatattctcctcaaaagtgtgtaaaaatccgacacacgagtagaatgggattgaagtgggtcaagatctgaaaccaatgggtcaatgccacaaaataaggtaaggtttctcgcagtagcaaaaaaataaagttaagttgagagaaaaattatgaatagttgtttctcgtgaagagttgtatacaaaaaaagtctaatttgatacattttcttttacatttataaaggattgaccaatcatccaaattaccaaatatttaggatataaatctttcagattacaacccatatatgatacactaaattttacatgaggcaatggaggatttgcatgaatatcgaggagagaaaaaattagttacaaaacttgcataatttatccaaaccaaatcaagtcaagaaacaacgaacaatattatcattagtactataagtatatattataggcttagagcaaagccctaactaccacactgcacacaaatgataactagtaagagaggaaaatacaaatttaagattcaacatagcaaattattcatgattcatgattcatgattcatgattcatgattcacgaacatcaagaatggtatagctgataaaggacaatttaaacataagtgtaaagctcgcacatcatcaattatattcgcatactactagaccaatctttacttagtacatgtgttagtacatgtgttacttcatatcagatgtattgattgttgccaatgacatatcatgttcacttaatcttagggccatttaattataacatggagaataatacaacttaaaattatgtggtggctatcatctcattttctagataattaaacctttattttgtatacatatatattgtctttacatagcaaaacaatattgaaggtataacaacctttcccttttcttttactacatgtttatgttagagtttttcgatttacgattgtggtaaattaattgtaattgatcggttgtcttgtagtcaagaaatgacgtatgaatcaatttagggcatgttctcttcggcataaaacagctgaactgaattgaactgaactgaaatgaatagtgatatgtgagagtaaaagtattgtcaagagctgaactgagctgaactgaacggatctgaactgaactgatctaatctgaactaatctgaactgaactgaactgaattgaactgaaaataagctagggaaaacagacccttactactattatataacctcgtttaaatattaggaaattaaaaaaataattatatttctttatactttattaacctattatacaatagtggtgatgtggccttggctccaatgggaccaaaatggaaaagaatgagaaggatatgcatggagcacttgctcacaactagacgacttgaactatttgtgagtcatagggctgatgaggcacgacatttggtccaagacgtattaactcgttcccacaaagataaagttgttaatttgagggaagtgttaggtgcattttctatgaataacgtgactagaatgttgctagggaagcaatactttggggccgggacggcgggcccacaagaggctctagagtttatgcatataacacatgagttgttttggttactaggcttgatttacttgggtgattatttgcctttttggaggtgggttgatccatatggatgtgaaaagaaaatgagggaagttgaaaaaagggtagatgatttccatcgcaaaattatagaggaacataggaaggagaagaaaaggaaagaagaaatgggagtgaatgagggtgaaatggattttgtagatattttgttggctttgcctggtgaaaatggaaatgagcatatggatgatgcagatattaaagctctaattcaggtaattcatgtataatttgaatgtgatacaaagtttgatagaaaacatatttgcataaatacatggttaccctactagacccaataaaatacataattattgccttgctagttgaaagttgaaacaacctagctaccattttgttgtgattatcattagccaaccaaaactatttcttgcatccatatattaatgttgagagtcgccatatttacaattacttgtaacattttgagcaaacaaattaaaatattttttggcaagtccattttattgaatgatacctatatcttaaaatgaatccttggttatgtacacttgcctttcaaggtaccaatatttgaccatatgtaattactattaacaaatttgataaaatctaataatatgtaaatatacgttcacgcacatattagaaacaaagatcacaaatgataatgcaaaataacttatttgaactaatgttgtgaagttaaatttggaacaaaaggtatatttgtattgccgaattttaatttataaattacttataacaacaactcaatatgtaaaactgttaagatggagtgtggatagaatgagatgagtatacttttactagttaccactcgaaattgcatttcctcctttgtttatagttgttctaacttctattatcataaataatttttggacttatttcaatgtatatttacaacgttaattgtttaattttttaaaaatacataatgtaaacaaggatttcatggtcaattatacacattatagatattatcttaaaaaacttactaatgctcaattagtgtccataatataggatatgatagcagcagcaacagacacatcagctgtaaccaacgaatgggccatggcagaagtaataaaacacccacgtgtcctccacaagatccaacaagagcttaacacaatagtaggacccaatcgaatggtaacagaatcagatcttccccaccttaactacctacgttgtgtcgtacgcgaaacgtttcggatgcatccagcaggaccctttttaatcccacatgaatcactacgccatacaacaatcaacggctatgatatcccatctgggacacgtgtcttcatcaacacacatgggttaggacgtaaccttaaagtgtgggacaacatagaggatttttaccctgaaagacattggccgttggatggaagtagagttgagattagccatggatctgattttaaaatattaccatttagtgctgggaagagaagatgtcctggggccccacttggggtggtgtttgtgttgatgggattggctacactttttcatgcatttgattggttaccacctgatggaatgaaggcagaagaaattgatactaaggaagtttatgggatgactatgcctaaagctcaacctttaatggctttggctaaacctaggcttgctcctcatttatatctttgatacatgttcatattgtggtgcacttataagcacactagctcaaatagacaaatacaagcttgtattgactctaacatgttatttaatattagcatactgcaactctacaactacaagtatgtaatctctataaattataaacataagtcataacgcatcttgttttgaaaaaaaaagctacactttcttacaccataaactgttccgttactaaattcttcttggcttattttagaattaatttttcaattttcctatttacttataacacaagataagaaaatcgactaacaatatttcacaacattatatcttgatagttcgttgaaattaataattttcaataatttcaaaacgcaaaaagcttctttgttcggataaattcacgtcattttcaataaaatcgcaaaatcttcccttgccatcaaggaacaaatttttcacatcaaaactcttcttccaaaaagtatacttctccattgattatgtaaaagagtttgcatgttctaatccattagtatgtatttttcggataagaagttgtattacaagcttaaataaagatataaacttagttaaatatcgaccaatccacaatatgtatcttcatatgtgtccatcagcgcctaaccttgagatgtagatcatctaaagcacattaacatggcgtactcctcctgttcaaatcagggttgtttgaaaccaaacttctccaaaatacttcctcagtaatgaagagaaaaactcacattctgaaaaacatcaacatcatggttctatggtctagtggttatgacactggactctgaatccagtaacccgagtt

SEQ ID NO: 77 - DNA, 4211 bp, Artificial Sequence
  BvCYP703A2_gst including artificial InDel marker allele
gtatatcgttgccacatgtgcgttgaatttttccttttcctatcctttccactccatattctcctcaaaagtgtgtaaaaatccgacacacgagtagaatgggattgaagtgggtcaagatctgaaaccaatgggtcaatgccacaaaataaggtaaggtttctcgcagtagcaaaaaaataaagttaagttgagagaaaaattatgaatagttgtttctcgtgaagagttgtatacaaaaaaagtctaatttgatacattttcttttacatttataaaggattgaccaatcatccaaattaccaaatatttaggatataaatctttcagattacaacccatatatgatacactaaattttacatgaggcaatggaggatttgcatgaatatcgaggagagaaaaaattagttacaaaacttgcataatttatccaaaccaaatcaagtcaagaaacaacgaacaatattatcattagtactataagtatatattataggcttagagcaaagccctaactaccacactgcacacaaatgataactagtaagagaggaaaatacaaatttaagattcaacatagcaaattattcatgattcatgattcatgattcatgattcatgattcacgaacatcaagaatggtatagctgataaaggacaatttaaacataagtgtaaagctcgcacatcatcaattatattcgcatactactagaccaatctttacttagtacatgtgttagtacatgtgttacttcatatcagatgtattgattgttgccaatgacatatcatgttcacttaatcttagggccatttaattataacatggagaataatacaacttaaaattatgtggtggctatcatctcattttctagataattaaacctttattttgtatacatatatattgtctttacatagcaaaacaatattgaaggtataacaacctttcccttttcttttactacatgtttatgttagagtttttcgatttacgattgtggtaaattaattgtaattgatcggttgtcttgtagtcaagaaatgacgtatgaatcaatttagggcatgttctcttcggcataaaacagctgaactgaattgaactgaactgaaatgaatagtgatatgtgagagtaaaagtattgtcaagagctgaactgagctgaactgaacggatctgaactgaactgatctaatctgaactaatctgaactgaactgaactgaattgaactgaaaataagctagggaaaacagacccttactactattatataacctcgtttaaatattaggaaattaaaaaaataattatatttctttatactttattaacctattatacaatagtggtgatgtggccttggctccaatgggaccaaaatggaaaagaatgagaaggatatgcatggagcacttgctcacaactagacgacttgaacttcatagggctgatgaggcacgacatttggtccaagacgtattaactcgttcccacaaagataaagttgttaatttgagggaagtgttaggtgcattttctatgaataacgtgactagaatgttgctagggaagcaatactttggggccgggacggcgggcccacaagaggctctagagtttatgcatataacacatgagttgttttggttactaggcttgatttacttgggtgattatttgcctttttggaggtgggttgatccatatggatgtgaaaagaaaatgagggaagttgaaaaaagggtagatgatttccatcgcaaaattatagaggaacataggaaggagaagaaaaggaaagaagaaatgggagtgaatgagggtgaaatggattttgtagatattttgttggctttgcctggtgaaaatggaaatgagcatatggatgatgcagatattaaagctctaattcaggtaattcatgtataatttgaatgtgatacaaagtttgatagaaaacatatttgcataaatacatggttaccctactagacccaataaaatacataattattgccttgctagttgaaagttgaaacaacctagctaccattttgttgtgattatcattagccaaccaaaactatttcttgcatccatatattaatgttgagagtcgccatatttacaattacttgtaacattttgagcaaacaaattaaaatattttttggcaagtccattttattgaatgatacctatatcttaaaatgaatccttggttatgtacacttgcctttcaaggtaccaatatttgaccatatgtaattactattaacaaatttgataaaatctaataatatgtaaatatacgttcacgcacatattagaaacaaagatcacaaatgataatgcaaaataacttatttgaactaatgttgtgaagttaaatttggaacaaaaggtatatttgtattgccgaattttaatttataaattacttataacaacaactcaatatgtaaaactgttaagatggagtgtggatagaatgagatgagtatacttttactagttaccactcgaaattgcatttcctcctttgtttatagttgttctaacttctattatcataaataatttttggacttatttcaatgtatatttacaacgttaattgtttaattttttaaaaatacataatgtaaacaaggatttcatggtcaattatacacattatagatattatcttaaaaaacttactaatgctcaattagtgtccataatataggatatgatagcagcagcaacagacacatcagctgtaaccaacgaatgggccatggcagaagtaataaaacacccacgtgtcctccacaagatccaacaagagcttaacacaatagtaggacccaatcgaatggtaacagaatcagatcttccccaccttaactacctacgttgtgtcgtacgcgaaacgtttcggatgcatccagcaggaccctttttaatcccacatgaatcactacgccatacaacaatcaacggctatgatatcccatctgggacacgtgtcttcatcaacacacatgggttaggacgtaaccttaaagtgtgggacaacatagaggatttttaccctgaaagacattggccgttggatggaagtagagttgagattagccatggatctgattttaaaatattaccatttagtgctgggaagagaagatgtcctggggccccacttggggtggtgtttgtgttgatgggattggctacactttttcatgcatttgattggttaccacctgatggaatgaaggcagaagaaattgatactaaggaagtttatgggatgactatgcctaaagctcaacctttaatggctttggctaaacctaggcttgctcctcatttatatctttgatacatgttcatattgtggtgcacttataagcacactagctcaaatagacaaatacaagcttgtattgactctaacatgttatttaatattagcatactgcaactctacaactacaagtatgtaatctctataaattataaacataagtcataacgcatcttgttttgaaaaaaaaagctacactttcttacaccataaactgttccgttactaaattcttcttggcttattttagaattaatttttcaattttcctatttacttataacacaagataagaaaatcgactaacaatatttcacaacattatatcttgatagttcgttgaaattaataattttcaataatttcaaaacgcaaaaagcttctttgttcggataaattcacgtcattttcaataaaatcgcaaaatcttcccttgccatcaaggaacaaatttttcacatcaaaactcttcttccaaaaagtatacttctccattgattatgtaaaagagtttgcatgttctaatccattagtatgtatttttcggataagaagttgtattacaagcttaaataaagatataaacttagttaaatatcgaccaatccacaatatgtatcttcatatgtgtccatcagcgcctaaccttgagatgtagatcatctaaagcacattaacatggcgtactcctcctgttcaaatcagggttgtttgaaaccaaacttctccaaaatacttcctcagtaatgaagagaaaaactcacattctgaaaaacatcaacatcatggttctatggtctagtggttatgacactggactctgaatccagtaacccgagtt

SEQ ID NO: 78 - DNA, 3252 bp, Beta vulgaris
atggcggctaccttcacaaacccaacattttccccttcctcaactccattaaccaaaaccctaaaatcccaatcttccatctcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggtgcatctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccaattgttgaggtgacaaggtctattactaagcataattatttagttttggatgtagaggatattcctagaattgttaaggaagccttttttttagctaattctggtaggcctggacctgttttgattgatcttcctaaagatattcagcagcaattggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgatcgtgtgaccgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaagtggagagaagaattaggtgagcagaagaaggaattcccactgagttttaagacatttggggatgcaattcctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaatgggaagataggttctataaagctaaccgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattgatcggtttaatgacggttggaaccatttaaagagggtaagctatattactgtatgtatattagtatgttcctggataatttagaagcttttgtctgttgtcttttgcagtttatgaagttagtttgctgttgtcatgttacttgttactttaaaaagctttttgtagtttttgagcaactagtatggaatgctcttcctgtattgcttggaaaattcacaaaagtggtttttcggctatggatgttgtgttgcatcatgcatatatagcttgatatactagttggcttggtgcatctttaacatatactaatgagactacgacagcaattgccaattagttggcttgacataattcttagtctgccactagaaatcttgcttcttttttttccctcatttgttgaaagtccctgttgcgacctgacatgggagcattggagattgtttagcaatagcagttgcagttaggtgactacagtcatcttccaaatatgaatactctctggaggggaggaggttttacaaaatatgagtttttaacacatgagaaacttatattaaacaaggttgagtcaccccatatttttcaaagttgacttttgtctgatttgggtgactatgcctgttgtatgcaataaactcgatgtacataagacttgtataatccaatctaacccttcctggctgattatgaaaccgagtcggctaatttgttgcttgatcttcatgtgtgagcctgatgccaggtgaccactagaggagtacctttcattgagatatcgattcggttaattggttcctcatatgggtctcaaaactgaatttttctcaggctctcttctaaccagttgttgaattttatgaaacttcagtccagttaaaactttgatcccggatcagaatttctctgagttgttcttccgcctctcagttccactgttccagtgttcttggtcctcaacctgtactctgagctatttctgcaaacacctgaagttcctgctgcccttggacaaatacagaatgcagcattagcatttatttaagacaagatgaatacttgctcccctgttagttatgctaattctgcattagtattctttaattttaattagatactgcaatccgtgaactctgcagtttcttgatctctctctgtttcaatttctcctatcttatcgcccatttctttaggctttcgttatctatcgtctaattcaagagtaggtaactaggtacgggataaaacttttt

SEQ ID NO: 79 - DNA, 3252 bp, Artificial Sequence
  BvALS_SU_res mutated BvALS W569L
atggcggctaccttcacaaacccaacattttccccttcctcaactccattaaccaaaaccctaaaatcccaatcttccatctcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggtgcatctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccaattgttgaggtgacaaggtctattactaagcataattatttagttttggatgtagaggatattcctagaattgttaaggaagccttttttttagctaattctggtaggcctggacctgttttgattgatcttcctaaagatattcagcagcaattggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgatcgtgtgaccgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaagtggagagaagaattaggtgagcagaagaaggaattcccactgagttttaagacatttggggatgcaattcctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaattggaagataggttctataaagctaaccgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattgatcggtttaatgacggttggaaccatttaaagagggtaagctatattactgtatgtatattagtatgttcctggataatttagaagcttttgtctgttgtcttttgcagtttatgaagttagtttgctgttgtcatgttacttgttactttaaaaagctttttgtagtttttgagcaactagtatggaatgctcttcctgtattgcttggaaaattcacaaaagtggtttttcggctatggatgttgtgttgcatcatgcatatatagcttgatatactagttggcttggtgcatctttaacatatactaatgagactacgacagcaattgccaattagttggcttgacataattcttagtctgccactagaaatcttgcttcttttttttccctcatttgttgaaagtccctgttgcgacctgacatgggagcattggagattgtttagcaatagcagttgcagttaggtgactacagtcatcttccaaatatgaatactctctggaggggaggaggttttacaaaatatgagtttttaacacatgagaaacttatattaaacaaggttgagtcaccccatatttttcaaagttgacttttgtctgatttgggtgactatgcctgttgtatgcaataaactcgatgtacataagacttgtataatccaatctaacccttcctggctgattatgaaaccgagtcggctaatttgttgcttgatcttcatgtgtgagcctgatgccaggtgaccactagaggagtacctttcattgagatatcgattcggttaattggttcctcatatgggtctcaaaactgaatttttctcaggctctcttctaaccagttgttgaattttatgaaacttcagtccagttaaaactttgatcccggatcagaatttctctgagttgttcttccgcctctcagttccactgttccagtgttcttggtcctcaacctgtactctgagctatttctgcaaacacctgaagttcctgctgcccttggacaaatacagaatgcagcattagcatttatttaagacaagatgaatacttgctcccctgttagttatgctaattctgcattagtattctttaattttaattagatactgcaatccgtgaactctgcagtttcttgatctctctctgtttcaatttctcctatcttatcgcccatttctttaggctttcgttatctatcgtctaattcaagagtaggtaactaggtacgggataaaacttttt

SEQ ID NO: 80 - DNA, 3261 bp, Artificial Sequence
  BvALS_SU_res including artifical InDel marker allele
atggcggctaccttcacaaacccaacattttccccttcctcaactccattaaccaaaaccctaaaatcccaatcttccatctcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggtgcatctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccaattgttgaggtgacaaggtctattactaagcataattatttagttttggatgtagaggatattcctagaattgttaaggaagccttttttttagctaattctggtaggcctggacctgttttgattgatcttcctaaagatattcagcagcaattggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgatcgtgtgaccgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaagtggagagaagaattaggtgagcagaagaaggaattcccactgagttttaagacatttggggatgcaattcctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaattggaagataggttctataaagctaaccgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattgatcggtttaatgacggttggaaccatttaaagagggtaagctatattactgtatgtatattagtatgttcctggataatttagaagcttttgtctgttgtcttttgcagtttatgaagttagtttgctgttgtcatgttacttgttactttaaaaagctttttgtagtttttgagcaactagtatggaatgctcttcctgtattgcttggaaaattcacaaaagtggtttttcggctatggatgttgtgttgcatcatgcatatatagcttgatatactagttggctgcactatcgtggtgcatctttaacatatactaatgagactacgacagcaattgccaattagttggcttgacataattcttagtctgccactagaaatcttgcttcttttttttccctcatttgttgaaagtccctgttgcgacctgacatgggagcattggagattgtttagcaatagcagttgcagttaggtgactacagtcatcttccaaatatgaatactctctggaggggaggaggttttacaaaatatgagtttttaacacatgagaaacttatattaaacaaggttgagtcaccccatatttttcaaagttgacttttgtctgatttgggtgactatgcctgttgtatgcaataaactcgatgtacataagacttgtataatccaatctaacccttcctggctgattatgaaaccgagtcggctaatttgttgcttgatcttcatgtgtgagcctgatgccaggtgaccactagaggagtacctttcattgagatatcgattcggttaattggttcctcatatgggtctcaaaactgaatttttctcaggctctcttctaaccagttgttgaattttatgaaacttcagtccagttaaaactttgatcccggatcagaatttctctgagttgttcttccgcctctcagttccactgttccagtgttcttggtcctcaacctgtactctgagctatttctgcaaacacctgaagttcctgctgcccttggacaaatacagaatgcagcattagcatttatttaagacaagatgaatacttgctcccctgttagttatgctaattctgcattagtattctttaattttaattagatactgcaatccgtgaactctgcagtttcttgatctctctctgtttcaatttctcctatcttatcgcccatttctttaggctttcgttatctatcgtctaattcaagagtaggtaactaggtacgggataaaacttttt